(19) United States
(12) Patent Application Publication
(10) Pub. No.: US 2024/0110202 A1
(43) Pub. Date: Apr. 4, 2024

(54) Advanced Cell-Permeable Peptide Carriers

(71) Applicant: MUSC FOUNDATION FOR RESEARCH DEVELOPMENT, Charleston, SC (US)

(72) Inventors: Andrew Jakymiw, Charleston, SC (US); Charles Holjencin, Summerville, SC (US)

(21) Appl. No.: 18/247,519

(22) PCT Filed: Oct. 4, 2021
(86) PCT No.: PCT/US21/53379

§ 371 Date: Mar. 31, 2023

Related U.S. Application Data
(60) Provisional application No. 63/086,823, filed on Oct. 2, 2020.

(51) Int. Cl. C12N 15/87; A61K 31/713; C12N 15/113

(52) U.S. Cl. CPC C12N 15/87; A61K 31/713; C12N 15/113; C12N 2310/14

(57) ABSTRACT

The present invention is generally related to compositions comprising and methods of using an optimized peptide delivery system for delivery of an agent into a desired cell. In certain aspects, the agent is a bioactive agent and the methods comprise the treatment or prevention of one or more disease or disorder.

### CROSS-REFERENCE TO RELATED APPLICATIONS

This application claims priority to U.S. Provisional Patent Application No. 63/086,823, filed on Oct. 2, 2020, the contents of which are incorporated by reference herein in its entirety.

### STATEMENT REGARDING FEDERALLY SPONSORED RESEARCH OR DEVELOPMENT

This invention was made with government support under Grant Nos. NIDCR R21DE027231 and NICDR T32DE017551 awarded by the National Institutes of Health (NIH). The government has certain rights in the invention.

### BACKGROUND OF THE INVENTION

One of the more recent promising avenues in cancer treatment has been in the use of RNA interference (RNAi), a highly conserved post-transcriptional gene silencing mechanism that involves the specific targeting of mRNA for degradation by small noncoding double-stranded RNA (dsRNA) molecules (Fire, et al. Nature, 1998. 391(6669):806-811; Rana. Nat Rev Mol Cell Biol, 2007. 8(1): 23-36). In fact, the discovery that the introduction of chemically synthesized small interfering RNAs (siRNAs) into cultured mammalian cells could efficiently induce sequence-specific gene silencing (Elbashir, et al. Nature, 2001. 411(6836): 494-498.), made evident the therapeutic potential of RNAi, as a means to specifically target and silence disease-causing genes (Cummings, et al. Transl Res, 2019. 214: 92-104). Since then, many studies have shown the therapeutic potential of RNAi, including various clinical trials, culminating in the world's first FDA-approved siRNA drug in 2018. Inclusive of these studies were preclinical and early phase clinical trials of human cancer patients that have demonstrated RNAi as a promising therapeutic tool for the treatment of cancer (Cummings, et al. Transl Res, 2019. 214: 92-104; Bobbin, et al. Annu Rev Pharmacol Toxicol, 2016. 56: 103-122; Das, et al. Nucleic Acid Ther, 2019. 29(2): 61-66; Zuckerman, et al. Nat Rev Drug Discov, 2015. 14(12): 843-856).

To maximize gene silencing efficiency, the therapeutic administration of siRNA requires a delivery platform that can overcome numerous challenges typically associated with this form of therapy, including, rapid renal excretion, degradation by RNases, homing to specific cell types, intracellular uptake, endosomal entrapment, and release of the siRNA cargo from the delivery platform (Cummings, et al. Transl Res, 2019. 214: 92-104; Whitehead, et al. Nat Rev Drug Discov, 2009. 8(2): 129-138; Gavrilov, et al. Yale J Biol Med, 2012. 85(2): 187-200). Currently, there are various types of siRNA delivery platforms, ranging from viral to non-viral vectors, each having their pros and cons when it comes to mitigating the obstacles described above, with the non-viral vectors encompassing technologies, such as lipids, polymers, aptamers, and peptides (Cummings, et al. Transl Res, 2019. 214: 92-104; Singh, et al. Artif Cells Nanomed Biotechnol, 2018. 46(2): 274-283; Marquez, et al. Oncomedicine, 2018. 3: 48-58). In particular, peptides, have been found to be a highly favorable drug carrier, due to the facile ability to control their design, which allows for the adjustment of function and physiochemical properties (Cummings, et al. Transl Res, 2019. 214: 92-104). In fact, when peptide delivery systems are designed properly, peptide-bound siRNA complexes are able to overcome many of the delivery challenges faced by naked siRNAs. One such peptide is the chimeric peptide 599, which was specifically designed to overcome challenges associated with siRNA cell uptake and endosomal entrapment through its cationic nona-arginine C-terminus and fusogenic INF7 N-terminus, respectively (Cantini, et al. PLoS One, 2013. 8(9): e73348). Together, these features led to the ability of 599 to enhance the intracellular delivery and bioavailability of therapeutic siRNAs designed to target the CIP2A oncogene (siCIP2A) into cancer cells in vitro and upon intratumoral injection in vivo, induce CIP2A silencing, resulting in the significant inhibition of tumor growth (Cantini, et al. PLoS One, 2013. 8(9): e73348; Alexander-Bryant, et al. J Control Release, 2015. 218: 72-81). In subsequent work, using a noncovalent multifunctional peptide complex approach, co-complexation of the 599 peptide with a cancer cell-targeting peptide was found to synergistically mediate the effective targeting/delivery of siCIP2As to xenograft oral cancer tumors upon systemic administration and significantly enhance CIP2A silencing (Alexander-Bryant, et al. Oral Oncol, 2017. 72: 123-131).

A common concern, however, for the therapeutic application of peptide carriers in vivo is their premature degradation prior to reaching their cell target for delivery of the drug cargo (Patel, et al. Pharm Res, 2007. 24(11): 1977-1992; Verdurmen, et al. Chem Biol, 2011. 18(8): 1000-1010). Therefore, a common strategy to combat this issue has been to modulate the stereochemistry of peptides, such that they comprise D-amino acids, thereby rendering them more protease resistant than their L-amino acid counterparts (Verdurmen, et al. Chem Biol, 2011. 18(8): 1000-1010; Elmquist, et al. Biol Chem, 2003. 384(3): 387-393; Pujals, et al. Biochem Soc Trans, 2007. 35(Pt 4): 794-796; Youngblood, et al. Bioconjug Chem, 2007. 18(1): 50-60), with increased stability not limited to peptides composed entirely of D-amino acids, but also observed for peptides with partial D-amino acid substitutions (Verdurmen, et al. Chem Biol, 2011. 18(8): 1000-1010; Youngblood, et al. Bioconjug Chem, 2007. 18(1): 50-60; Tugyi, et al. Proc Natl Acad Sci USA, 2005. 102(2): 413-418). In fact, the nona-arginine tract in 599 was designed to contain D-arginines for this particular reason and shown to be able to protect siRNAs from degradation by serum and ribonucleases in vitro and upon intratumoral injection in vivo (Cantini, et al. PLoS One, 2013. 8(9): e73348; Alexander-Bryant, et al. J Control Release, 2015. 218: 72-81). Although the inclusion of D-arginines within the design of peptide carriers may confer protease resistance, the inclusion of L-arginines also appears to have its advantages. Interestingly, in assessing the effects of attaching cell-binding ligands to arginine-rich cell-penetrating peptides (CPPs), Zeller et al. found that ligands attached to L-arginine-rich CPPs, but not D-arginine-rich CPPs, could prolong the dynamics of gene silencing due to the enhanced release of siRNAs from late endosomes to the cytosol (Zeller, et al. Chem Biol, 2015. 22(1): 50-62). This effect was demonstrated to be dependent on endosomal proteolytic activity, which implied that partial degradation of the arginine-rich CPP was necessary for endosome-to-cytosol translocation of the siRNAs. Moreover, this effect could also explain why another study using an analogous approach connecting a tumor-targeting peptide with a D-arginine-rich tract for delivery of siRNAs showed no knockdown of the targeted gene, even in the presence of an endosomolytic peptide (Jun, et al. PLoS One, 2015. 10(2): e0118310). Only when a selected arginine was replaced with an alanine, thought in-part to reduce the electrostatic interactions between the peptide and siRNA, was gene silencing induced, suggesting the need again for effective release of siRNAs from the peptides (Jun, et al. PLoS One, 2015. 10(2): e0118310).

Thus, there is a need in the art for improved compositions and methods for delivery of siRNA and RNAi-related molecules to cells. This invention satisfies this unmet need.

### BRIEF SUMMARY OF THE INVENTION

In one embodiment, the present invention relates to a composition for delivery of an agent comprising: a) a peptide, wherein the peptide comprises a derivative of 599 peptide (SEQ ID NO: 1); and b) an agent.

In one embodiment of the composition, the peptide comprising a derivative of SEQ ID NO: 1 comprises an amino acid sequence in which at least one D-arginine residue of SEQ ID NO: 1 is substituted with an alanine residue. In one embodiment, at least one D-arginine residue of SEQ ID NO: 1 is substituted with a D-alanine residue. In one embodiment, at least one D-arginine residue of SEQ ID NO: 1 is substituted with a D-alanine residue at an amino acid residue position number selected from the group consisting of: 27, 28, 29, 30, 31, 32, 33, 34, and 35.

In one embodiment, the peptide comprises an amino acid sequence selected from the group consisting of SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, and SEQ ID NO: 32.

In one embodiment of the composition, the peptide comprising a derivative of SEQ ID NO: 1 comprises an amino acid sequence in which at least one D-arginine residue of SEQ ID NO: 1 is substituted with an L-alanine residue. In one embodiment, at least one D-arginine residue of SEQ ID NO: 1 is substituted with an L-alanine residue at an amino acid residue position number selected from the group consisting of: 27, 28, 29, 30, 31, 32, 33, 34, and 35. In one embodiment, the peptide comprises an amino acid sequence selected from the group consisting of SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 34, SEQ ID NO: 35, SEQ ID NO: 36, SEQ ID NO: 37, SEQ ID NO: 38, SEQ ID NO: 39, and SEQ ID NO: 40.

In one embodiment of the composition, the peptide comprising a derivative of SEQ ID NO: 1 comprises an amino acid sequence in which at least one D-arginine residue is substituted with an L-arginine residue.

In one embodiment of the composition, the peptide comprising a derivative of SEQ ID NO: 1 comprises an amino acid sequence in which at least three D-arginine residues are each substituted with an L-arginine residue. In one embodiment, the peptide comprises an amino acid sequence selected from the group consisting of: SEQ ID NO: 2, SEQ ID NO: 3, SEQ ID NO: 4, SEQ ID NO: 5, SEQ ID NO: 6, SEQ ID NO: 8, SEQ ID NO: 9, SEQ ID NO: 10, SEQ ID NO: 11, SEQ ID NO: 12, SEQ ID NO: 13, and SEQ ID NO: 15.

In one embodiment of the composition, the peptide comprising a derivative of SEQ ID NO: 1 comprises an amino acid sequence in which at least one L-form residue of SEQ ID NO: 1 is substituted with the D-form of said residue. In one embodiment, the peptide comprises an amino acid sequence selected from the group consisting of SEQ ID NO: 7 and SEQ ID NO: 14.

In one embodiment of the composition, the agent is selected from the group consisting of: a nucleic acid molecule, a protein, a peptide, an antibody, an antibody fragment, and a small molecule. In one embodiment, the nucleic acid molecule is selected from the group consisting of: siRNA, microRNA, shRNA, antisense nucleic acid, ribozyme, killer-tRNA, guide RNA (part of the CRISPR/CAS system), long non-coding RNA, anti-miRNA oligonucleotide, mRNA, and plasmid DNA.

In one embodiment of the composition, the peptide and agent are at a molar ratio between about 5:1 to 50:1. In one embodiment of the composition, said peptide comprises a polyethylene glycol (PEG) modification to prevent renal clearance.

In one embodiment, the composition further comprises a second peptide comprising a targeting moiety and a stretch of densely packed cationic amino acid residues. In one embodiment, the second peptide comprises an amino acid sequence of SEQ ID NO: 41.

In one embodiment, the present invention relates to a method of administering an agent into a cell, the method comprising contacting the cell with an effective amount of a composition comprising: a) a peptide, wherein the peptide comprises a derivative of 599 peptide (SEQ ID NO: 1); and b) an agent.

In one embodiment, the present invention relates to a method of treating a disease or disorder in a subject in need thereof, the method comprising administering to the subject a therapeutically effective amount of any one of the compositions of the present invention. In one embodiment, the method comprises administering to the subject a therapeutically effective amount of a composition comprising: a) a peptide, wherein the peptide comprises a derivative of 599 peptide (SEQ ID NO: 1); and b) an agent. In one embodiment, the method comprises administering to the subject a therapeutically effective amount of a composition comprising: a) a peptide, wherein the peptide comprises a derivative of 599 peptide (SEQ ID NO: 1); b) an agent; and c) a second peptide comprising a targeting moiety and a stretch of densely packed cationic amino acid residues. In one embodiment, the agent alleviates at least one symptom of the disease or disorder. In one embodiment, the disease or disorder is cancer.

In one embodiment, the present invention comprises a composition for delivery of an agent comprising: a) a peptide, wherein the peptide comprises 599 peptide (SEQ ID NO: 1); and b) an agent, wherein the agent comprises one or more selected from the group consisting of: plasmid DNA and mRNA.

In one embodiment, the present invention comprises a method of administering an agent to a cell, the method comprising contacting the cell with an effective amount of a composition comprising: a) a peptide, wherein the peptide comprises 599 peptide (SEQ ID NO: 1); and b) an agent, wherein the agent comprises one or more selected from the group consisting of: plasmid DNA and mRNA.

In one embodiment, the present invention comprises a method of treating a disease or disorder in a subject in need thereof, the method comprising administering to the subject a therapeutically effective amount of a composition comprising: a) a peptide, wherein the peptide comprises 599 peptide (SEQ ID NO: 1); and b) an agent, wherein the agent comprises one or more selected from the group consisting of: plasmid DNA and mRNA.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1 depicts the results of exemplary experiments depicting siRNA binding analysis of 599 and its peptide variants at increasing peptide:siRNA molar ratios. Densitometric quantitation of siCIP2A levels complexed to 599 or its peptide variants at increasing Peptide:siRNA molar (nitrogen:phosphate [N/P]) ratios. Data are mean±SEM of three independent samples, where *p<0.05, **p<0.01, ***p<0.001, ****p<0.0001, and p≥0.05 is not significant (ns) compared to 599 (2-way ANOVA).

FIG. 2, comprising FIG. 2A and FIG. 2B, depicts the results of exemplary experiments demonstrating siRNA delivery into CAL 27 cancer cells via 599 and its peptide variants. FIG. 2A depicts exemplary quantitative siRNA uptake into CAL 27 cancer cells 2 h post-treatment with DY547-labeled siCIP2A (DY547-siCIP2A) in complex with 599 or its peptide variants at either the 30:1 or 50:1 Peptide:siRNA molar (7.5 or 12.5 N/P) ratios. For comparison, the cells were also transfected using the commercial lipid-based transfection reagent (LTR), Lipofectamine 3000 (LF3000). The amount of siRNA delivered into cells in pmol per mg of protein is reported with each treatment normalized to DY547-siCIP2A alone. Data are mean±SEM of three independent samples, where *p<0.05, **p<0.01, and p≥0.05 is not significant (ns) compared to 599 (2-way ANOVA). FIG. 2B depicts a representative confocal fluorescence microscopy analysis of CAL 27 cancer cells 2 h post-treatment with DY547-CIP2A (red) in complex with 599 or its peptide variants at 50:1 Peptide:siRNA molar (12.5 N/P) ratios. Nuclei (blue) were counterstained with DAPI. The arrowheads indicate highly ordered linear punctate uptake patterns of siRNAs in the RD3AD panel. Scale bar: 35 μm.

FIG. 3, comprising FIG. 3A and FIG. 3B, depicts the results of exemplary experiments demonstrating the assessment of cellular toxicity of CAL 27 cells 48 hours post-treatment with 599 and its peptide variants in complex with siRNAs. The long-term cellular toxicity (as measured by a cell proliferation assay) of CAL 27 cancer cells was assessed 48 h post-treatment with 599 or its peptide variants in complex with a non-targeting siRNA (siNT) at 50:1 Peptide:siRNA molar (12.5 NIP) ratios. FIG. 3A depicts a comparison to untreated cells. Untreated cells were defined as 100% viable and data are mean±SEM of three independent samples, where p≥0.05 is not significant (ns) compared to untreated cells (1-way ANOVA). FIG. 3B depicts a comparison to 599 peptide-treated cells, where 599-peptide treated cells are defined as 100% viable. Data are mean±SEM of three independent samples, where p≥0.05 is not significant (ns) compared to 599 (1-way ANOVA).

FIG. 4 depicts the results of exemplary experiments depicting RNase and serum protection analyses of 599 and its peptide variants in complex with siRNAs. Naked and peptide-complexed siCIP2As at a 0:1 and 50:1 Peptide:siRNA molar (0 and 12.5 N/P) ratio, respectively, were incubated in the absence or presence of either RNase A or 50% human serum for 1 h at 37° C., after which densitometric quantitation was performed to determine the levels of protected siRNAs. Data are mean±SEM of three independent samples, where *p<0.05, **p<0.01, ***p<0.001, ****p<0.0001, andp≥0.05 is not significant compared to 599 (Student's t test).

FIG. 5 depicts results of exemplary experiments depicting an siRNA release analysis from 599 and its peptide variants. Densitometric quantitation of released siRNAs from 599 and its peptide variants complexed at the 50:1 Peptide:siRNA molar (12.5 N/P) ratio upon addition of increasing amounts of heparin (0-10 mg). Data are mean±SEM of three independent samples, where *p<0.05, **p<0.01, ***p<0.001, ****p<0.0001, and p≥0.05 is not significant (ns) compared to 599 (2-way ANOVA).

FIG. 6, comprising FIG. 6A, FIG. 6B, and FIG. 6C, depicts results of exemplary experiments depicting CIP2A gene silencing mediated by 599 and its peptide variants in complex with siCIP2A. FIG. 6A depicts an exemplary real-time PCR analysis of CIP2A mRNA levels in CAL 27 cancer cells 48 h post-treatment with siNT or siCIP2A in complex with either 599 or its peptide variants at 50:1 Peptide:siRNA molar (12.5 N/P) ratios. The CIP2A mRNA levels were normalized to endogenous 18S rRNA. Data are mean±SEM of three independent samples, where *p<0.05, **p<0.01, ***p<0.001, ****p<0.0001, and p≥0.05 is not significant compared to siNT-treated cells (Student's t test). FIG. 6B depicts exemplary results of fold change in CIP2A silencing mediated by the 599 peptide variants normalized to 599. Data are mean±SEM of three independent samples, where **p<0.01, ***p<0.001, and p≥0.05 is not significant (ns) compared to 599 (Student's t test). FIG. 6C depicts exemplary Western blot analyses of CIP2A protein expression levels in CAL 27 cancer cells 48 h post-treatment with siNT or siCIP2A in complex with either 599 or its peptide variants at 50:1 Peptide:siRNA molar (12.5 N/P) ratios. β-actin protein levels were monitored to ensure equal loading of samples.

FIG. 7, comprising FIG. 7A, FIG. 7B, and FIG. 7C, depicts results of exemplary experiments depicting confocal fluorescence microscopy analysis of DY547-labeled siCIP2A cellular localization mediated by 599 and its peptide variants. FIG. 7A depicts a representative confocal fluorescence microscopy analysis of CAL 27 cancer cells 2 h post-treatment with DY547-siCIP2A (red) in complex with 599 or its peptide variants at 50:1 Peptide: siRNA molar (12.5 N/P) ratios. Filopodia (green) were stained with the selective F-actin label Alexa Fluor 488 phalloidin and nuclei (blue) were counterstained with DAPI. Scale bar: 17 mm. FIG. 7B depicts representative confocal fluorescence microscopy images of 599 and RD3AD-mediated localization of DY547-siCIP2A in CAL 27 cancer cells from FIG. 7A separated into their individual fluorophore signals. The merged images are also presented. Scale bar: 17 μm. FIG. 7C depicts a representative confocal fluorescence microscopy analysis of SCC-90 cancer cells 2 h post-treatment with DY547-siCIP2A (red) in complex with 599 or RD3AD at a 50:1 Peptide:siRNA molar (12.5 N/P) ratio. Filopodia (green) were stained with the selective F-actin label Alexa Fluor 488 phalloidin and nuclei (blue) were counterstained with DAPI. Scale bar: 27 μm.

FIG. 8 depicts exemplary time-course analyses of siRNA cellular localization mediated by RD3AD in comparison to 599. Confocal fluorescence microscopy analyses of CAL 27 cancer cells 0.5, 1, 2, and 4 h post-treatment with DY547-siCIP2A (red) in complex with 599 or RD3AD at a 50:1 Peptide:siRNA molar (12.5 N/P) ratio. Alexa Fluor 488 phalloidin was used to stain the F-actin (green) in filopodia, as well as the cytoplasm of cells, so as to help demarcate the cell bodies. Nuclei (blue) were counterstained with DAPI. The upper-half panels represent merged images of the red and blue fluorophore signals only, while the lower-half panels represent all three (red, blue, and green) fluorophore signals combined. Cross-sectional views comprising z sections along the x and y planes are also presented within each panel. Yellow fluorescence reveals the potential colocalization between siRNA and F-actin. Scale bar: 70 μm.

FIG. 9, comprising FIG. 9A and FIG. 9B, depicts exemplary results demonstrating that the siRNA cell localization and gene silencing mediated by RD3AD occurs independently of the chirality of the substituted alanine residue within its peptide design. FIG. 9A depicts a representative confocal fluorescence microscopy analysis of CAL 27 oral cancer cells 2 hours post-treatment with DY547-siCIP2A (red) in complex with RD3AD or its peptide variant RD3AL at 50:1 peptide:siRNA molar (12.5 N/P) ratios. Nuclei (blue) were counterstained with DAPI. Scale bar: 15 μm. FIG. 9B depicts an exemplary qPCR analysis of CIP2A mRNA levels in CAL 27 cancer cells 48 hours post-treatment with siNT or siCIP2A in complex with either RD3AD or RD3AL at 50:1 peptide:siRNA molar (12.5 N/P) ratios. The CIP2A mRNA levels were normalized to endogenous 18S rRNA. Data are mean±SEM of three independent biological samples, where ****P<0.0001 compared to siNT-treated cells and p≥0.05 is not significant (ns) compared to RD3AD-siCIP2A complex-treated cells (Student's t test).

FIG. 10, comprising FIG. 10A and FIG. 10B, depicts exemplary results demonstrating that disruption of filopodia interactions and structures impairs the ability of RD3AD to deliver siRNAs to cells. FIG. 10A depicts a representative confocal fluorescence microscopy analysis of CAL 27 cancer cells pre-treated for 24 hours with/without 35 mM sodium chlorate (NaClO₃) and then subsequently treated for 2 hours with DY547-siCIP2A (red) in complex with 599 or RD3AD at 50:1 peptide:siRNA molar (12.5 N/P) ratios. FIG. 10B depicts a representative fluorescence microscopy analysis of CAL 27 cancer cells pre-treated for 1 hour with/without 10 μM SMIFH2 and then subsequently treated for 2 hours with DY547-siCIP2A (red) in complex with RD3AD at a 50:1 peptide:siRNA molar (12.5 N/P) ratio. For FIG. 10A and FIG. 10B, filopodia (green) were stained with the CellMask™ Green Actin Tracking Stain and nuclei (blue) were counterstained with DAPI. Scale bars: 15 μm.

FIG. 11, comprising FIG. 11A, FIG. 11B, and FIG. 11C, depicts the results of exemplary experiments comparing the effects of 599 peptide and RD3AD used in conjunction with GE11R9 as part of a dual peptide siRNA delivery system. FIG. 11A depicts exemplary results demonstrating knockdown of CIP2A mRNA in CAL 27 cancer cells when using RD3AD in conjunction with GE11R9, as compared to 599 and GE11R9. FIG. 11B depicts exemplary results demonstrating a greater fold change in gene silencing when using RD3AD in conjunction with GE11R9 as compared to 599 and GE11R9. FIG. 11C depicts exemplary results demonstrating the specificity of GE11R9, as both RD3AD are 599 are able to knockdown CIP2A in CAL 27 cells more efficiently when used in conjunction with GE11R9 as compared to a scrambled control peptide, GE 11SCR9.

FIG. 12, comprising FIG. 12A, FIG. 12B, FIG. 12C and FIG. 12D, depicts exemplary results demonstrating that 599 peptide can interact comparably with additional siRNA species. FIG. 12A depicts results of exemplary experiments comparing the percentage of 599 peptide binding to siCIP2A and siDicer1 at increasing peptide:siRNA molar ratios. FIG. 12B depicts results of exemplary experiments demonstrating the percentage of 599 peptide binding to siE6 at increasing peptide:siRNA molar ratios. FIG. 12C depicts results of exemplary experiments demonstrating the percentage of release of siE6 from 599 peptide at increasing heparin concentrations. FIG. 12D depicts results of exemplary experiments demonstrating protection of siE6 from RNAse A by 599 peptide at a peptide:siRNA molar ratio of 50:1.

FIG. 13 depicts an exemplary peptide binding assay for 599 peptide and a representative plasmid DNA construct. 599 peptide was complexed with phrGFP-N1 plasmid DNA 4.3 kb in length at increasing peptide:DNA molar ratios, resolved on a 1% agarose gel and visualized with a UV detection system. Bound DNA fails to migrate past the loading well, while unbound DNA can migrate freely through the gel.

FIG. 14 depicts an exemplary cell uptake assay for 599 peptide and a representative plasmid DNA construct. AD293 cells were treated with plasmid DNA encoding a GFP reporter protein (phrGFP-N1) complexed to the 599 peptide at a 5,000:1 peptide:DNA molar ratio. 72 hours post-treatment, expression of the GFP reporter protein (green) encoded by phrGFP-N1 was evaluated by fluorescence microscopy.

FIG. 15 depicts an exemplary peptide binding assay for 599 peptide and a representative mRNA molecule encoding GFP. 599 peptide was complexed with 500 ng of mRNA (1300 nucleotides in length) at increasing peptide:mRNA (N/P) molar ratios, resolved on a 2% agarose gel and visualized with a UV detection system. Bound mRNA fails to migrate past the loading well, while unbound mRNA can migrate freely through the gel.

FIG. 16 depicts an exemplary peptide release assay for 599 peptide and a representative mRNA molecule. 500 ng of mRNA encoding GFP was complexed to 599 peptide at a phosphate:nitrogen molar ratio of 12.5 (8.8 μg), treated with increasing concentrations of heparin to compete for binding, resolved on a 2% agarose gel, and visualized with a UV detection system. Bound mRNA fails to migrate past the loading well, while free mRNA can migrate freely through the gel.

### DETAILED DESCRIPTION

The present invention relates to compositions and methods for the delivery of an agent into a desired cell. For example, in certain aspects, the invention comprises a composition of a peptide that efficiently interacts with the agent and mediates the delivery of the agent into the cell. The present invention is based on the discovery that modifying patterns of amino acid stereochemistry and/or specific amino acid substitutions within a peptide can improve the delivery and release of a bioactive agent into a desired cell. The invention also relates to methods of making and using such a peptide, methods of delivering an agent, and methods of treating or preventing a disease or disorder using said methods of delivery.

### Definitions

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

“Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double-stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences.

The term “anti-tumor effect” as used herein, refers to a biological effect which can be manifested by a decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of the peptides, polynucleotides, cells, and antibodies of the invention in prevention of the occurrence of tumor in the first place.

As used herein, the term “bind” or “binding” refers to the specific association or other specific interaction between two molecular species, such as, but not limited to, protein-DNA/RNA interactions and protein-protein interactions, for example, the specific association between proteins and their DNA/RNA targets, receptors and their ligands, enzymes and their substrates, etc. Such binding may be specific or non-specific, and can involve various noncovalent interactions, such as hydrogen bonding, metal coordination, hydrophobic forces, van der Waals forces, pi-pi interactions, and/or electrostatic effects.

The term “cancer” as used herein is defined as disease characterized by the rapid and uncontrolled growth of aberrant cells. Cancer cells can spread locally or through the bloodstream and lymphatic system to other parts of the body. Examples of various cancers include, but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer, oral cancer and the like.

The terms “cells” and “population of cells” are used interchangeably and refer to a plurality of cells, i.e., more than one cell. The population may be a pure population comprising one cell type. Alternatively, the population may comprise more than one cell type. In the present invention, there is no limit on the number of cell types that a cell population may comprise.

The term “cell-penetrating peptide” refers to a peptide that, when contacted with the extracellular side of the cell membrane or cell wall of a cell, enters the intracellular region of the cell.

As used herein, the terms “complementary” or “complementarity” are used in reference to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

An “effective amount” as used herein, means an amount which provides a therapeutic or prophylactic benefit.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “expression” as used herein is defined as the transcription and/or translation of a particular nucleotide sequence driven by its promoter

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

As used herein “endogenous” refers to any material from or produced inside an organism, cell, tissue or system.

As used herein, the term “exogenous” refers to any material introduced from or produced outside an organism, cell, tissue or system.

As used herein, the term “fragment,” as applied to a nucleic acid, refers to a subsequence of a larger nucleic acid. A “fragment” of a nucleic acid can be at least about 36 nucleotides in length; for example, at least about 40 nucleotides to about 50 nucleotides; at least about 50 to about 60 nucleotides, at least about 60 to about 70 nucleotides; at least about 70 nucleotides to about 80 nucleotides; about 80 nucleotides to about 90 nucleotides; or about 100 nucleotides (and any integer value in between). As used herein, the term “fragment,” as applied to a protein or peptide, refers to a subsequence of a larger protein or peptide. A “fragment” of a protein or peptide can be at least about 12 amino acids in length; for example, a fragment of SEQ ID NO:1 can be at least about 12, 13, 14, 15, 16, 17, 18, 19, 20, 21, 22, 23, 24, or 25 amino acids in length.

The term “functionally equivalent” as used herein refers to a polypeptide according to the invention that retains at least one biological function or activity of the specific amino acid sequence of either the first or second peptide.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared×100. For example, if 6 of 10 positions in two sequences are matched or homologous then the two sequences are 60% homologous. By way of example, the DNA sequences ATTGCC and TATGGC share 50% homology. Generally, a comparison is made when two sequences are aligned to give maximum homology.

The phrase “inhibit,” as used herein, means to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and mRNA stability, expression, function and activity, e.g., antagonists.

“Instructional material,” as that term is used herein, includes a publication, a recording, a diagram, or any other medium of expression which can be used to communicate the usefulness of the composition or method of the invention. Optionally, or alternately, the instructional material may describe one or more methods of identifying, diagnosing or alleviating the diseases or disorders in a cell or a tissue of a subject. The instructional material of the kit may, for example, be affixed to a container that contains the composition of the invention or be shipped together with a container that contains the composition. Alternatively, the instructional material may be shipped separately from the container with the intention that the recipient uses the instructional material and the composition cooperatively.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “label” when used herein refers to a detectable compound or composition that is conjugated directly or indirectly to a molecule to generate a “labeled” molecule. The label may be detectable by itself (e.g. radioisotope labels or fluorescent labels) or, in the case of an enzymatic label, may catalyze chemical alteration of a substrate compound or composition that is detectable (e.g., avidin-biotin).

The term “miRNA” is used according to its ordinary and plain meaning and refers to a microRNA molecule found in eukaryotes that is involved in RNA-based gene regulation. See, e.g., Carrington et al., 2003, which is hereby incorporated by reference. The term will be used to refer to the single-stranded RNA molecule processed from a precursor. Individual miRNAs have been identified and sequenced in different organisms, and they have been given names. Names of miRNAs and their sequences are provided herein. Additionally, other miRNAs are known to those of skill in the art and can be readily implemented in embodiments of the invention. The methods and compositions should not be limited to miRNAs identified in the application, as they are provided as examples, not necessarily as limitations of the invention.

By the term “modulating,” as used herein, is meant mediating a detectable increase or decrease in the level of a mRNA, polypeptide, or a response in a subject, or a cell or tissue of a subject, as compared with the level of a mRNA, polypeptide or a response in the subject, or a cell or tissue of the subject, in the absence of a treatment or compound, and/or compared with the level of a mRNA, polypeptide, or a response in an otherwise identical, but untreated subject, or cell or tissue of the subject. The term encompasses perturbing and/or affecting a native signal or response thereby mediating a beneficial therapeutic response in a subject, for example, a human.

A “nucleic acid” refers to a polynucleotide and includes poly-ribonucleotides and poly-deoxyribonucleotides. Nucleic acids according to the present invention may include any polymer or oligomer of pyrimidine and purine bases, for example, cytosine, thymine, and uracil, and adenine and guanine, respectively. (See Albert L. Lehninger, Principles of Biochemistry, at 793-800 (Worth Pub. 1982), which is herein incorporated in its entirety for all purposes). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, 8, 15 or 25 nucleotides in length, but may be up to 50, 100, 1000, or 5000 nucleotides long or a compound that specifically hybridizes to a polynucleotide. Polynucleotides include sequences of deoxyribonucleic acid (DNA) or ribonucleic acid (RNA) or mimetics thereof which may be isolated from natural sources, recombinantly produced or artificially synthesized. A further example of a polynucleotide of the present invention may be a peptide nucleic acid (PNA). (See U.S. Pat. No. 6,156,501 which is hereby incorporated by reference in its entirety.) The invention also encompasses situations in which there is a nontraditional base pairing such as Hoogsteen base pairing, which has been identified in certain tRNA molecules and postulated to exist in a triple helix. “Polynucleotide” and “oligonucleotide” are used interchangeably in this disclosure. It will be understood that when a nucleotide sequence is represented herein by a DNA sequence (e.g., A, T, G, and C), this also includes the corresponding RNA sequence (e.g., A, U, G, C) in which “U” replaces “T”.

The term “overexpressed” tumor antigen or “overexpression” of the tumor antigen is intended to indicate an abnormal level of expression of the tumor antigen in a cell from a disease area like a solid tumor within a specific tissue or organ of the patient relative to the level of expression in a normal cell from that tissue or organ. Patients having solid tumors or a hematological malignancy characterized by overexpression of the tumor antigen can be determined by standard assays known in the art.

The terms “patient,” “subject,” “individual,” and the like are used interchangeably herein, and refer to any animal, or cells thereof whether in vitro or in situ, amenable to the methods described herein. In certain non-limiting embodiments, the patient, subject, or individual is a human.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, “polynucleotide” includes cDNA, RNA, DNA/RNA hybrid, antisense RNA, ribozyme, genomic DNA, synthetic forms, and mixed polymers, both sense and antisense strands, and may be chemically or biochemically modified to contain non-natural or derivatized, synthetic, or semi-synthetic nucleotide bases. Also, contemplated are alterations of a wild type or synthetic gene, including, but not limited to deletion, insertion, substitution of one or more nucleotides, or fusion to other polynucleotide sequences.

As used herein, the terms “ribonucleotide,” “oligoribonucleotide,” and “polyribonucleotide” refers to a string of at least 2 base-sugar-phosphate combinations. The term includes, in another embodiment, compounds comprising nucleotides in which the sugar moiety is ribose. In another embodiment, the term includes both RNA and RNA derivates in which the backbone is modified. “Nucleotides” refers, in another embodiment, to the monomeric units of nucleic acid polymers. RNA may be, in an other embodiment, in the form of a tRNA (transfer RNA), snRNA (small nuclear RNA), rRNA (ribosomal RNA), mRNA (messenger RNA), anti-sense RNA, small interfering/small inhibitory RNA (siRNA), micro RNA (miRNA) and ribozymes. The use of siRNA and miRNA has been described (Caudy A A et al., Genes & Devel 16: 2491-96 and references cited therein). In addition, these forms of RNA may be single, double, triple, or quadruple stranded. The term also includes, in another embodiment, artificial nucleic acids that may contain other types of backbones, but the same bases. In another embodiment, the artificial nucleic acid is a PNA (peptide nucleic acid). PNA contain peptide backbones and nucleotide bases and are able to bind, in another embodiment, to both DNA and RNA molecules. In another embodiment, the nucleotide is oxetane modified. In another embodiment, the nucleotide is modified by replacement of one or more phosphodiester bonds with a phosphorothioate bond. In another embodiment, the artificial nucleic acid contains any other variant of the phosphate backbone of native nucleic acids known in the art. The use of phosphothiorate nucleic acids and PNA are known to those skilled in the art, and are described in, for example, Neilsen P E, Curr Opin Struct Biol 9:353-57; and Raz N K et al. Biochem Biophys Res Commun. 297:1075-84. The production and use of nucleic acids is known to those skilled in art and is described, for example, in Molecular Cloning, (2012), Sambrook and Green, eds. and Methods in Enzymology: Methods for molecular cloning in eukaryotic cells (2003) Purchio and G. C. Fareed. Each nucleic acid derivative represents a separate embodiment of the present invention

By the term “specifically binds,” as used herein, is meant a molecule, such as an antibody or peptide, which recognizes and binds to another molecule or feature, but does not substantially recognize or bind other molecules or features in a sample.

As used herein, the terms “therapy” or “therapeutic regimen” refer to those activities taken to alleviate or alter a disorder or disease state, e.g., a course of treatment intended to reduce or eliminate at least one sign or symptom of a disease or disorder using pharmacological, surgical, dietary and/or other techniques. A therapeutic regimen may include a prescribed dosage of one or more drugs or surgery. Therapies will most often be beneficial and reduce or eliminate at least one sign or symptom of the disorder or disease state, but in some instances the effect of a therapy will have non-desirable or side-effects. The effect of therapy will also be impacted by the physiological state of the subject, e.g., age, gender, genetics, weight, other disease or disorder conditions, etc.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease or disorder and its severity and the age, weight, etc., of the subject to be treated.

To “treat” a disease or disorder as the term is used herein, means to reduce the frequency or severity of at least one sign or symptom of a disease or disorder experienced by a subject.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range, such as from 1 to 6 should be considered to have specifically disclosed subranges, such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention generally relates to a peptide delivery system that can be used as a carrier for the delivery of an agent to a desired cell. For example, in one embodiment, the invention relates to a composition comprising a peptide that has cell penetrating capabilities, the ability to bind to an agent, and the ability to release the agent once inside the cell. In certain embodiments, the peptide is modified to improve these favorable characteristics. For example, in one embodiment, the peptide comprises a stretch of cationic residues interrupted with one or more nonpolar residues to improve binding to a bioactive reagent and possibly release upon cellular uptake. In other embodiments, the peptide comprises a stretch of cationic residues comprising a mixture of D and L cationic amino acids.

In some embodiments, the composition comprises an agent to be delivered by the peptide. For example, in one embodiment, the agent is a bioactive agent, where the agent is capable of modulating a desired biological function or activity. In one embodiment, the bioactive agent modulates endogenous protein expression within the desired cell. For example, the agent may be siRNA, which can reduce mRNA expression of a target gene to in turn reduce translated protein levels. In one embodiment, the agent comprises an exogenous nucleic acid that is endogenously expressed by the cell. For example, the agent may be mRNA or plasmid DNA to increase expression of an endogenously expressed protein. In other embodiments, the bioactive agent is used to treat a disease or disorder. For example, the bioactive agent can have anti-tumor activity to treat cancer.

In certain embodiments, peptides of the present invention can be combined with one or more targeting peptide in a dual peptide system to target specific cells or cell features.

### Compositions

In one aspect, the present invention relates to a composition comprising a peptide for delivery of an agent to a target cell. Therefore, in one embodiment, the invention relates to compositions comprising a combination of (1) one or more peptide that exhibits endosome-disruptive activity for delivery of an agent and (2) one or more agent of interest.

In certain embodiments, the peptide comprises a stretch of densely packed cationic amino acid residues. Non-limiting examples of cationic amino acid residues include arginine, lysine and histidine. In some instances, the stretch of densely packed cationic amino acid residues includes at least four arginine residues. In some instances, at least one of the arginine residues is a D-form stereoisomer. In some instances, at least one arginine residue is a D-form stereoisomer, and at least one arginine residue is a L-form stereoisomer. In some instances, the stretch of cationic amino acid residues is interrupted by at least one nonpolar residue. In some instances, the nonpolar residue is alanine. In certain instances, the alanine is D-alanine.

In certain embodiments, the peptide has cell penetrating capabilities. In some instances, the peptide can bind to and deliver the agent to a desired cell. In some instances, the peptide can release the agent once inside the cell. In one embodiment, the peptide has endosome-disruptive activity to promote release of the agent into the cytosol of the desired cell. In one embodiment, the agent is a bioactive agent having biological function or activity. For example, in one embodiment, the bioactive agent functions to alleviate the symptoms of a disease or disorder. In certain embodiments, the disease or disorder is cancer.

In some embodiments, the peptide comprises the 599 peptide (SEQ ID NO: 1). In some embodiments, the peptide comprises a derivative of the 599 peptide (SEQ ID NO: 1). Exemplary derivatives of the 599 peptide are shown in the table below.

| | | SEQ ID |
|---|---|---|
| Peptide name | Peptide sequence^{a,b} | NO: |
| 599 | GLFEAIEGFIENGWEGMIDGWYGGGGrrrrrrrrrK | 1 |
| RD456 | GLFEAIEGFIENGWEGMIDGWYGGGGRRRrrrRRRK | 2 |
| All-L | GLFEAIEGFIENGWEGMIDGWYGGGGRRRRRRRRRK | 3 |
| RD19 | GLFEAIEGFIENGWEGMIDGWYGGGGrRRRRRRRrK | 4 |
| RD123789 | GLFEAIEGFIENGWEGMIDGWYGGGGrrrRRRrrrK | 5 |
| RD13579 | GLFEAIEGFIENGWEGMIDGWYGGGGrRrRrRrRrK | 6 |
| All-D | GlfeaieGfienGweGmidGwyGGGGrrrrrrrrrk | 7 |
| INF7D | GlfeaieGfienGweGmidGwyGGGGRRRRRRRRRK | 8 |
| RD456/3AD | GLFEAIEGFIENGWEGMIDGWYGGGGRRarrrRRRK | 9 |
| All-L/3AD | GLFEAIEGFIENGWEGMIDGWYGGGGRRaRRRRRRK | 10 |
| RD19/3AD | GLFEAIEGFIENGWEGMIDGWYGGGGrRaRRRRRrK | 11 |
| RD12789/3AD | GLFEAIEGFIENGWEGMIDGWYGGGGrraRRRrrrK | 12 |
| RD1579/3AD | GLFEAIEGFIENGWEGMIDGWYGGGGrRaRrRrRrK | 13 |
| All-D/3AD | GlfeaieGfienGweGmidGwyGGGGrrarrrrrrk | 14 |
| INF7D/3AD | GlfeaieGfienGweGmidGwyGGGGRRaRRRRRRK | 15 |
| RD1AD | GLFEAIEGFIENGWEGMIDGWYGGGGarrrrrrrrK | 16 |
| RD1AL | GLFEAIEGFIENGWEGMIDGWYGGGGArrrrrrrrK | 17 |
| RD2AD | GLFEAIEGFIENGWEGMIDGWYGGGGrarrrrrrrK | 18 |
| RD2AL | GLFEAIEGFIENGWEGMIDGWYGGGGrArrrrrrrK | 19 |
| RD3AD | GLFEAIEGFIENGWEGMIDGWYGGGGrrarrrrrrK | 20 |
| RD3AL | GLFEAIEGFIENGWEGMIDGWYGGGGrrArrrrrrK | 21 |
| RD4AD | GLFEAIEGFIENGWEGMIDGWYGGGGrrrarrrrrK | 22 |
| RD4AL | GLFEAIEGFIENGWEGMIDGWYGGGGrrrArrrrrK | 23 |
| RD5AD | GLFEAIEGFIENGWEGMIDGWYGGGGrrrrarrrrK | 24 |
| RD5AL | GLFEAIEGFIENGWEGMIDGWYGGGGrrrrArrrrK | 25 |
| RD6AD | GLFEAIEGFIENGWEGMIDGWYGGGGrrrrrarrrK | 26 |
| RD6AL | GLFEAIEGFIENGWEGMIDGWYGGGGrrrrrArrrK | 27 |
| RD7AD | GLFEAIEGFIENGWEGMIDGWYGGGGrrrrrrarrK | 28 |
| RD7AL | GLFEAIEGFIENGWEGMIDGWYGGGGrrrrrrArrK | 29 |
| RD8AD | GLFEAIEGFIENGWEGMIDGWYGGGGrrrrrrrark | 30 |
| RD8AL | GLFEAIEGFIENGWEGMIDGWYGGGGrrrrrrrArK | 31 |
| RD9AD | GLFEAIEGFIENGWEGMIDGWYGGGGrrrrrrrraK | 32 |
| RD9AL | GLFEAIEGFIENGWEGMIDGWYGGGGrrrrrrrrAK | 33 |
| RD456/3AL | GLFEAIEGFIENGWEGMIDGWYGGGGRRArrrRRRK | 34 |
| All-L/3AL | GLFEAIEGFIENGWEGMIDGWYGGGGRRARRRRRRK | 35 |
| RD19/3AL | GLFEAIEGFIENGWEGMIDGWYGGGGrRARRRRRrK | 36 |
| RD12789/3AL | GLFEAIEGFIENGWEGMIDGWYGGGGrrARRRrrrK | 37 |
| RD1579/3AL | GLFEAIEGFIENGWEGMIDGWYGGGGrRARrRrRrK | 38 |
| All-D/3AL | GlfeaieGfienGweGmidGwyGGGGrrArrrrrrk | 39 |
| INF7D/3AL | GlfeaieGfienGweGmidGwyGGGGRRARRRRRRK | 40 |

ªD-amino acids are indicated by a lower case script letter; L-amino acids are capitalized; G remains capitalized always, as glycine has no stereochemistry.
^{b}Substituted arginine residues for alanine are underlined.

In some embodiments, the peptide of the present invention comprises any one of SEQ ID NOs: 1-40. In some embodiments, the peptide of the present invention comprises any one of SEQ ID NOs: 2-40.

In one embodiment, the peptide comprises a derivative of SEQ ID NO: 1, wherein at least six of the nine residues in the cationic stretch of residues of SEQ ID NO: 1 are modified to L-form. In one embodiment, the peptide comprises a derivative of SEQ ID NO: 1, wherein the D-arginine residues at positions 27, 28, 29, 33, 34 and 35 of SEQ ID NO: 1 are modified to L-arginine. In one embodiment, the peptide comprises the sequence: GLFEAIEGFIENGWEGMIDGWYGGGGRRRrrrRRRK (SEQ ID NO: 2), wherein lowercase letters refer to D-amino acids and wherein capital letters refer to L-amino acids.

In one embodiment, the peptide comprises a derivative of SEQ ID NO: 1, wherein at least nine of the nine residues in the cationic stretch of residues of SEQ ID NO: Iare modified to L-form. In one embodiment, the peptide comprises a derivative of SEQ ID NO: 1, wherein the D-arginine residues at positions 27-35 of SEQ ID NO: 1 are modified to L-arginine residues. In one embodiment, the peptide comprises the sequence: GLFEAIEGFIENGWEGMIDGWYGGGGRRRRRRRRRK (SEQ ID NO: 3), wherein capital letters refer to L-amino acids.

In one embodiment, the peptide comprises a derivative of SEQ ID NO: 1, wherein at least seven of the nine residues in the cationic stretch of residues of SEQ ID NO: 1 are modified to L-form. In one embodiment, the peptide comprises a derivative of SEQ ID NO: 1, wherein the D-arginine residues at positions 28-34 of SEQ ID NO: 1 are modified to L-arginine residues. In one embodiment, the peptide comprises the sequence: GLFEAIEGFIENGWEGMIDGWYGGGGrRRRRRRRrK (SEQ ID NO: 4), wherein lowercase letters refer to D-amino acids and wherein capital letters refer to L-amino acids.

In one embodiment, the peptide comprises a derivative of SEQ ID NO: 1, wherein at least three of the nine residues in the cationic stretch of residues of SEQ ID NO: 1 are modified to L-form. In one embodiment, the peptide comprises a derivative of SEQ ID NO: 1, wherein the D-arginine residues at positions 30, 31 and 32 of SEQ ID NO: 1 are modified to L-arginine residues. In one embodiment, the peptide comprises the sequence: GLFEAIEGFIENGWEGMIDGWYGGGGrrrRRRrrrK (SEQ ID NO: 5), wherein lowercase letters refer to D-amino acids and wherein capital letters refer to L-amino acids.

In one embodiment, the peptide comprises a derivative of SEQ ID NO: 1, wherein at least four of the nine residues in the cationic stretch of residues of SEQ ID NO: 1 are modified to L-form. In one embodiment, the peptide comprises a derivative of SEQ ID NO: 1, wherein the D-arginine residues at positions 28, 30, 32, and 34 of SEQ ID NO: 1 are modified to L-arginine residues. In one embodiment, the peptide comprises the sequence: GLFEAIEGFIENGWEGMIDGWYGGGGrRrRrRrRrK (SEQ ID NO: 6), wherein lowercase letters refer to D-amino acids and wherein capital letters refer to L-amino acids.

In one embodiment, the peptide comprises a derivative of SEQ ID NO: 1, wherein at least one or more of the residues of SEQ ID NO: 1 that are L-form are modified to D-form residues. In one embodiment, the peptide comprises a derivative of SEQ ID NO: 1, wherein all of the L-form residues of SEQ ID NO: 1 are modified to D-form residues. In one embodiment, the peptide comprises the sequence: GlfeaieGfienGweGmidGwyGGGGrrrrrrrrrk (SEQ ID NO: 7), wherein lowercase letters refer to D-amino acids.

In one embodiment, the peptide comprises a derivative of SEQ ID NO: 1, wherein at least one or more of the residues of SEQ ID NO: 1 that are L-form residues are modified to D-form residues, and wherein at least one or more of the residues within the stretch of cationic residues of SEQ ID NO: 1 is modified to L-form. In one embodiment, the peptide comprises a derivative of SEQ ID NO: 1, wherein all of the L-form residues of SEQ ID NO: 1 are modified to D-form residues, and wherein all of the residues within the stretch of cationic residues of SEQ ID NO: 1 are modified to L-form.

In one embodiment, the peptide comprises the sequence: GlfeaieGfienGweGmidGwyGGGGRRRRRRRRRK (SEQ ID NO: 8), wherein lowercase letters refer to D-amino acids and wherein capital letters refer to L-amino acids.

In one embodiment, the peptide comprises a derivative of SEQ ID NO: 1, wherein at least five of the nine residues in the cationic stretch of residues of SEQ ID NO: 1 are modified to L-form, and wherein at least one arginine residue of SEQ ID NO: 1 is substituted with alanine. In one embodiment, the peptide comprises a derivative of SEQ ID NO: 1, wherein the D-arginine residues at positions 27, 28, 33, 34 and 35 of SEQ ID NO: 1 are modified to L-arginine residues, and wherein the D-arginine at position 29 is substituted for D-alanine. In one embodiment, the peptide comprises the sequence: GLFEAIEGFIENGWEGMIDGWYGGGGRRarrrRRRK (SEQ ID NO: 9), wherein lowercase letters refer to D-amino acids and wherein capital letters refer to L-amino acids.

In one embodiment, the peptide comprises a derivative of SEQ ID NO: 1, wherein at least eight of the nine residues in the cationic stretch of residues of SEQ ID NO: 1 are modified to L-form, and wherein at least one arginine residue of SEQ ID NO: 1 is substituted with alanine. In one embodiment, the peptide comprises a derivative of SEQ ID NO: 1, wherein the D-arginine residues at positions 27, 28, and 30-35 of SEQ ID NO: 1 are modified to L-arginine residues, and wherein the D-arginine at position 29 is substituted for D-alanine. In one embodiment, the peptide comprises the sequence: GLFEAIEGFIENGWEGMIDGWYGGGGRRaRRRRRRK (SEQ ID NO: 10), wherein lowercase letters refer to D-amino acids and wherein capital letters refer to L-amino acids.

In one embodiment, the peptide comprises a derivative of SEQ ID NO: 1, wherein at least six of the nine residues in the cationic stretch of residues of SEQ ID NO: 1 are modified to L-form, and wherein at least one arginine residue of SEQ ID NO: 1 is substituted with alanine. In one embodiment, the peptide comprises a derivative of SEQ ID NO: 1, wherein the D-arginine residues at positions 28, and 30-34 of SEQ ID NO: 1 are modified to L-arginine residues, and wherein the D-arginine at position 29 is substituted for D-alanine. In one embodiment, the peptide comprises the sequence: GLFEAIEGFIENGWEGMIDGWYGGGGrRaRRRRRrK (SEQ ID NO: 11), wherein lowercase letters refer to D-amino acids and wherein capital letters refer to L-amino acids.

In one embodiment, the peptide comprises a derivative of SEQ ID NO: 1, wherein at least three of the nine residues in the cationic stretch of residues of SEQ ID NO: 1 are modified to L-form, and wherein at least one arginine residue of SEQ ID NO: 1 is substituted with alanine. In one embodiment, the peptide comprises a derivative of SEQ ID NO: 1, wherein the D-arginine residues at positions 30, 31 and 32 of SEQ ID NO: 1 are modified to L-arginine residues, and wherein the D-arginine at position 29 is substituted for D-alanine. In one embodiment, the peptide comprises the sequence: GLFEAIEGFIENGWEGMIDGWYGGGGrraRRRrrrK (SEQ ID NO: 12), wherein lowercase letters refer to D-amino acids and wherein capital letters refer to L-amino acids.

In one embodiment, the peptide comprises a derivative of SEQ ID NO: 1, wherein at least four of the nine residues in the cationic stretch of residues of SEQ ID NO: 1 are modified to L-form, and wherein at least one arginine residue of SEQ ID NO: 1 is substituted with alanine. In one embodiment, the peptide comprises a derivative of SEQ ID NO: 1, wherein the D-arginine residues at positions 28, 30, 32, and 34 of SEQ ID NO: 1 are modified to L-arginine residues, and wherein the D-arginine at position 29 is substituted for D-alanine. In one embodiment, the peptide comprises the sequence: GLFEAIEGFIENGWEGMIDGWYGGGGrRaRrRrRrK (SEQ ID NO: 13), wherein lowercase letters refer to D-amino acids and wherein capital letters refer to L-amino acids.

In one embodiment, the peptide comprises a derivative of SEQ ID NO: 1, wherein at least one or more of the residues of SEQ ID NO: 1 that are L-form are modified to D-form residues, and wherein at least one arginine residue of SEQ ID NO: 1 is substituted with alanine. In one embodiment, the peptide comprises a derivative of SEQ ID NO: 1, wherein all of the L-form residues of SEQ ID NO: 1 are modified to D-form residues, and wherein the D-arginine at position 29 is substituted for D-alanine. In one embodiment, the peptide comprises the sequence: GlfeaieGfienGweGmidGwyGGGGrrarrrrrrk (SEQ ID NO: 14), wherein lowercase letters refer to D-amino acids.

In one embodiment, the peptide comprises a derivative of SEQ ID NO: 1, wherein at least one or more of the residues of SEQ ID NO: 1 that are L-form residues are modified to D-form residues, wherein at least one or more of the residues within the stretch of cationic residues SEQ ID NO: 1 is modified to L-form, and wherein at least one arginine residue of SEQ ID NO: 1 is substituted with alanine. In one embodiment, the peptide comprises a derivative of SEQ ID NO: 1, wherein all of the L-form residues of SEQ ID NO: 1 are modified to D-form residues, wherein eight of the nine residues within the stretch of cationic residues of SEQ ID NO: 1 are modified to L-form, and wherein the D-arginine at position 29 is substituted for D-alanine. In one embodiment, the peptide comprises the sequence: GlfeaieGfienGweGmidGwyGGGGRRaRRRRRRK (SEQ ID NO: 15), wherein lowercase letters refer to D-amino acids and wherein capital letters refer to L-amino acids.

In one embodiment, the peptide comprises a derivative of SEQ ID NO: 1, wherein at least one D-arginine residue of SEQ ID NO: 1 is substituted with D-alanine. In one embodiment, the peptide comprises a derivative of SEQ ID NO: 1, wherein the D-arginine residue at position 27 of SEQ ID NO: 1 is substituted with D-alanine. In one embodiment, the peptide comprises the sequence: GLFEAIEGFIENGWEGMIDGWYGGGGarrrrrrrrK (SEQ ID NO: 16), wherein lowercase letters refer to D-amino acids and wherein capital letters refer to L-amino acids.

In one embodiment, the peptide comprises a derivative of SEQ ID NO: 1, wherein at least one D-arginine residue of SEQ ID NO: 1 is substituted with L-alanine. In one embodiment, the peptide comprises a derivative of SEQ ID NO: 1, wherein the D-arginine residue at position 27 of SEQ ID NO: 1 is substituted with L-alanine. In one embodiment, the peptide comprises the sequence: GLFEAIEGFIENGWEGMIDGWYGGGGArrrrrrrrK (SEQ ID NO: 17), wherein lowercase letters refer to D-amino acids and wherein capital letters refer to L-amino acids.

In one embodiment, the peptide comprises a derivative of SEQ ID NO: 1, wherein at least one D-arginine residue of SEQ ID NO: 1 is substituted with D-alanine. In one embodiment, the peptide comprises a derivative of SEQ ID NO: 1, wherein the D-arginine residue at position 28 of SEQ ID NO: 1 is substituted with D-alanine. In one embodiment, the peptide comprises the sequence: GLFEAIEGFIENGWEGMIDGWYGGGGrarrrrrrrK (SEQ ID NO: 18), wherein lowercase letters refer to D-amino acids and wherein capital letters refer to L-amino acids.

In one embodiment, the peptide comprises a derivative of SEQ ID NO: 1, wherein at least one D-arginine residue of SEQ ID NO: 1 is substituted with L-alanine. In one embodiment, the peptide comprises a derivative of SEQ ID NO: 1, wherein the D-arginine residue at position 28 of SEQ ID NO: 1 is substituted with L-alanine. In one embodiment, the peptide comprises the sequence: GLFEAIEGFIENGWEGMIDGWYGGGGrArrrrrrrK (SEQ ID NO: 19), wherein lowercase letters refer to D-amino acids and wherein capital letters refer to L-amino acids.

In one embodiment, the peptide comprises a derivative of SEQ ID NO: 1, wherein at least one D-arginine residue of SEQ ID NO: 1 is substituted with D-alanine. In one embodiment, the peptide comprises a derivative of SEQ ID NO: 1, wherein the D-arginine residue at position 29 of SEQ ID NO: 1 is substituted with D-alanine. In one embodiment, the peptide comprises the sequence: GLFEAIEGFIENGWEGMIDGWYGGGGrrarrrrrrK (SEQ ID NO: 20), wherein lowercase letters refer to D-amino acids and wherein capital letters refer to L-amino acids.

In one embodiment, the peptide comprises a derivative of SEQ ID NO: 1, wherein at least one D-arginine residue of SEQ ID NO: 1 is substituted with L-alanine. In one embodiment, the peptide comprises a derivative of SEQ ID NO: 1, wherein the D-arginine residue at position 29 of SEQ ID NO: 1 is substituted with L-alanine. In one embodiment, the peptide comprises the sequence: GLFEAIEGFIENGWEGMIDGWYGGGGrrArrrrrrK (SEQ ID NO: 21), wherein lowercase letters refer to D-amino acids and wherein capital letters refer to L-amino acids.

In one embodiment, the peptide comprises a derivative of SEQ ID NO: 1, wherein at least one D-arginine residue of SEQ ID NO: 1 is substituted with D-alanine. In one embodiment, the peptide comprises a derivative of SEQ ID NO: 1, wherein the D-arginine residue at position 30 of SEQ ID NO: 1 is substituted with D-alanine. In one embodiment, the peptide comprises the sequence: GLFEAIEGFIENGWEGMIDGWYGGGGrrrarrrrrK (SEQ ID NO: 22), wherein lowercase letters refer to D-amino acids and wherein capital letters refer to L-amino acids.

In one embodiment, the peptide comprises a derivative of SEQ ID NO: 1, wherein at least one D-arginine residue of SEQ ID NO: 1 is substituted with L-alanine. In one embodiment, the peptide comprises a derivative of SEQ ID NO: 1, wherein the D-arginine residue at position 30 of SEQ ID NO: 1 is substituted with L-alanine. In one embodiment, the peptide comprises the sequence: GLFEAIEGFIENGWEGMIDGWYGGGGrrrArrrrrK (SEQ ID NO: 23), wherein lowercase letters refer to D-amino acids and wherein capital letters refer to L-amino acids.

In one embodiment, the peptide comprises a derivative of SEQ ID NO: 1, wherein at least one D-arginine residue of SEQ ID NO: 1 is substituted with D-alanine. In one embodiment, the peptide comprises a derivative of SEQ ID NO: 1, wherein the D-arginine residue at position 31 of SEQ ID NO: 1 is substituted with D-alanine. In one embodiment, the peptide comprises the sequence: GLFEAIEGFIENGWEGMIDGWYGGGGrrrrarrrrK (SEQ ID NO: 24), wherein lowercase letters refer to D-amino acids and wherein capital letters refer to L-amino acids.

In one embodiment, the peptide comprises a derivative of SEQ ID NO: 1, wherein at least one D-arginine residue of SEQ ID NO: 1 is substituted with L-alanine. In one embodiment, the peptide comprises a derivative of SEQ ID NO: 1, wherein the D-arginine residue at position 31 of SEQ ID NO: 1 is substituted with L-alanine. In one embodiment, the peptide comprises the sequence: GLFEAIEGFIENGWEGMIDGWYGGGGrrrrArrrrK (SEQ ID NO: 25), wherein lowercase letters refer to D-amino acids and wherein capital letters refer to L-amino acids.

In one embodiment, the peptide comprises a derivative of SEQ ID NO: 1, wherein at least one D-arginine residue of SEQ ID NO: 1 is substituted with D-alanine. In one embodiment, the peptide comprises a derivative of SEQ ID NO: 1, wherein the D-arginine residue at position 32 of SEQ ID NO: 1 is substituted with D-alanine. In one embodiment, the peptide comprises the sequence: GLFEAIEGFIENGWEGMIDGWYGGGGrrrrrarrrK (SEQ ID NO: 26), wherein lowercase letters refer to D-amino acids and wherein capital letters refer to L-amino acids.

In one embodiment, the peptide comprises a derivative of SEQ ID NO: 1, wherein at least one D-arginine residue of SEQ ID NO: 1 is substituted with L-alanine. In one embodiment, the peptide comprises a derivative of SEQ ID NO: 1, wherein the D-arginine residue at position 32 of SEQ ID NO: 1 is substituted with L-alanine. In one embodiment, the peptide comprises the sequence: GLFEAIEGFIENGWEGMIDGWYGGGGrrrrrArrrK (SEQ ID NO: 27), wherein lowercase letters refer to D-amino acids and wherein capital letters refer to L-amino acids.

In one embodiment, the peptide comprises a derivative of SEQ ID NO: 1, wherein at least one D-arginine residue of SEQ ID NO: 1 is substituted with D-alanine. In one embodiment, the peptide comprises a derivative of SEQ ID NO: 1, wherein the D-arginine residue at position 33 of SEQ ID NO: 1 is substituted with D-alanine. In one embodiment, the peptide comprises the sequence: GLFEAIEGFIENGWEGMIDGWYGGGGrrrrrrarrK (SEQ ID NO: 28), wherein lowercase letters refer to D-amino acids and wherein capital letters refer to L-amino acids.

In one embodiment, the peptide comprises a derivative of SEQ ID NO: 1, wherein at least one D-arginine residue of SEQ ID NO: 1 is substituted with L-alanine. In one embodiment, the peptide comprises a derivative of SEQ ID NO: 1, wherein the D-arginine residue at position 33 of SEQ ID NO: 1 is substituted with L-alanine. In one embodiment, the peptide comprises the sequence: GLFEAIEGFIENGWEGMIDGWYGGGGrrrrrrArrK (SEQ ID NO: 29), wherein lowercase letters refer to D-amino acids and wherein capital letters refer to L-amino acids.

In one embodiment, the peptide comprises a derivative of SEQ ID NO: 1, wherein at least one D-arginine residue of SEQ ID NO: 1 is substituted with D-alanine. In one embodiment, the peptide comprises a derivative of SEQ ID NO: 1, wherein the D-arginine residue at position 34 of SEQ ID NO: 1 is substituted with D-alanine. In one embodiment, the peptide comprises the sequence: GLFEAIEGFIENGWEGMIDGWYGGGGrrrrrrrarK (SEQ ID NO: 30), wherein lowercase letters refer to D-amino acids and wherein capital letters refer to L-amino acids.

In one embodiment, the peptide comprises a derivative of SEQ ID NO: 1, wherein at least one D-arginine residue of SEQ ID NO: 1 is substituted with L-alanine. In one embodiment, the peptide comprises a derivative of SEQ ID NO: 1, wherein the D-arginine residue at position 34 of SEQ ID NO: 1 is substituted with L-alanine. In one embodiment, the peptide comprises the sequence: GLFEAIEGFIENGWEGMIDGWYGGGGrrrrrrrArK (SEQ ID NO: 31), wherein lowercase letters refer to D-amino acids and wherein capital letters refer to L-amino acids.

In one embodiment, the peptide comprises a derivative of SEQ ID NO: 1, wherein at least one D-arginine residue of SEQ ID NO: 1 is substituted with D-alanine. In one embodiment, the peptide comprises a derivative of SEQ ID NO: 1, wherein the D-arginine residue at position 35 of SEQ ID NO: 1 is substituted with D-alanine. In one embodiment, the peptide comprises the sequence: GLFEAIEGFIENGWEGMIDGWYGGGGrrrrrrrraK (SEQ ID NO: 32), wherein lowercase letters refer to D-amino acids and wherein capital letters refer to L-amino acids.

In one embodiment, the peptide comprises a derivative of SEQ ID NO: 1, wherein at least one D-arginine residue of SEQ ID NO: 1 is substituted with L-alanine. In one embodiment, the peptide comprises a derivative of SEQ ID NO: 1, wherein the D-arginine residue at position 35 of SEQ ID NO: 1 is substituted with L-alanine. In one embodiment, the peptide comprises the sequence: GLFEAIEGFIENGWEGMIDGWYGGGGrrrrrrrrAK (SEQ ID NO: 33), wherein lowercase letters refer to D-amino acids and wherein capital letters refer to L-amino acids.

In one embodiment, the peptide comprises a derivative of SEQ ID NO: 1, wherein at least six of the nine residues in the cationic stretch of residues of SEQ ID NO: 1 are modified to L-form, and wherein at least one arginine residue of SEQ ID NO: 1 is substituted with alanine. In one embodiment, the peptide comprises a derivative of SEQ ID NO: 1, wherein the D-arginine residues at positions 27, 28, 33, 34 and 35 of SEQ ID NO: 1 are modified to L-arginine residues, and wherein the D-arginine at position 29 is substituted for L-alanine. In one embodiment, the peptide comprises the sequence: GLFEAIEGFIENGWEGMIDGWYGGGGRRArrrRRRK (SEQ ID NO: 34), wherein lowercase letters refer to D-amino acids and wherein capital letters refer to L-amino acids.

In one embodiment, the peptide comprises a derivative of SEQ ID NO: 1, wherein at least nine residues in the cationic stretch of residues of SEQ ID NO: 1 are modified to L-form, and wherein at least one arginine residue of SEQ ID NO: 1 is substituted with alanine. In one embodiment, the peptide comprises a derivative of SEQ ID NO: 1, wherein the D-arginine residues at positions 27, 28, and 30-35 of SEQ ID NO: 1 are modified to L-arginine residues, and wherein the D-arginine at position 29 is substituted for L-alanine. In one embodiment, the peptide comprises the sequence: GLFEAIEGFIENGWEGMIDGWYGGGGRRARRRRRRK (SEQ ID NO: 35), wherein capital letters refer to L-amino acids.

In one embodiment, the peptide comprises a derivative of SEQ ID NO: 1, wherein at least seven of the nine residues in the cationic stretch of residues of SEQ ID NO: 1 are modified to L-form, and wherein at least one arginine residue of SEQ ID NO: 1 is substituted with alanine. In one embodiment, the peptide comprises a derivative of SEQ ID NO: 1, wherein the D-arginine residues at positions 28, and 30-34 of SEQ ID NO: 1 are modified to L-arginine residues, and wherein the D-arginine at position 29 is substituted for L-alanine. In one embodiment, the peptide comprises the sequence: GLFEAIEGFIENGWEGMIDGWYGGGGrRARRRRRrK (SEQ ID NO: 36), wherein lowercase letters refer to D-amino acids and wherein capital letters refer to L-amino acids.

In one embodiment, the peptide comprises a derivative of SEQ ID NO: 1, wherein at least four of the nine residues in the cationic stretch of residues of SEQ ID NO: 1 are modified to L-form, and wherein at least one arginine residue of SEQ ID NO: 1 is substituted with alanine. In one embodiment, the peptide comprises a derivative of SEQ ID NO: 1, wherein the D-arginine residues at positions 30, 31 and 32 of SEQ ID NO: 1 are modified to L-arginine residues, and wherein the D-arginine at position 29 is substituted for L-alanine. In one embodiment, the peptide comprises the sequence: GLFEAIEGFIENGWEGMIDGWYGGGGrrARRRrrrK (SEQ ID NO: 37), wherein lowercase letters refer to D-amino acids and wherein capital letters refer to L-amino acids.

In one embodiment, the peptide comprises a derivative of SEQ ID NO: 1, wherein at least five of the nine residues in the cationic stretch of residues of SEQ ID NO: 1 are modified to L-form, and wherein at least one arginine residue of SEQ ID NO: 1 is substituted with alanine. In one embodiment, the peptide comprises a derivative of SEQ ID NO: 1, wherein the D-arginine residues at positions 28, 30, 32, and 34 of SEQ ID NO: 1 are modified to L-arginine residues, and wherein the D-arginine at position 29 is substituted for L-alanine. In one embodiment, the peptide comprises the sequence: GLFEAIEGFIENGWEGMIDGWYGGGGrRARrRrRrK (SEQ ID NO: 38), wherein lowercase letters refer to D-amino acids and wherein capital letters refer to L-amino acids.

In one embodiment, the peptide comprises a derivative of SEQ ID NO: 1, wherein at least one or more of the residues of SEQ ID NO: 1 that are L-form are modified to D-form residues, and wherein at least one arginine residue of SEQ ID NO: 1 is substituted with alanine. In one embodiment, the peptide comprises a derivative of SEQ ID NO: 1, wherein all of the L-form residues of SEQ ID NO: 1 are modified to D-form residues, and wherein the D-arginine at position 29 is substituted for L-alanine. In one embodiment, the peptide comprises the sequence: GlfeaieGfienGweGmidGwyGGGGrrArrrrrrk (SEQ ID NO: 39), wherein lowercase letters refer to D-amino acids and wherein capital letters refer to L-amino acids.

In one embodiment, the peptide comprises a derivative of SEQ ID NO: 1, wherein at least one or more of the residues of SEQ ID NO: 1 that are L-form residues are modified to D-form residues, wherein at least one or more of the residues within the stretch of cationic residues SEQ ID NO: 1 is modified to L-form, and wherein at least one arginine residue of SEQ ID NO: 1 is substituted with alanine. In one embodiment, the peptide comprises a derivative of SEQ ID NO: 1, wherein all of the L-form residues of SEQ ID NO: 1 are modified to D-form residues, wherein all residues within the stretch of cationic residues of SEQ ID NO: 1 are modified to L-form, and wherein the D-arginine at position 29 is substituted for L-alanine. In one embodiment, the peptide comprises the sequence: GlfeaieGfienGweGmidGwyGGGGRR_RRRRRRK (SEQ ID NO: 40), wherein lowercase letters refer to D-amino acids and wherein capital letters refer to L-amino acids.

In one embodiment, the peptide is between 10 and 100 amino acids in length. In one embodiment, the peptide is between 20 and 50 amino acids in length. In one embodiment, the peptide is between 30 and 40 amino acids in length. In one embodiment, the peptide is about 36 amino acids in length.

In one aspect, the present invention provides a composition comprising a peptide comprising a polyarginine tract, or derivative thereof, as described herein. For example, in one embodiment, the peptide comprises a polyarginine tract comprising at least four arginine residues. In some instances, at least one of the arginine residues is a D-form stereoisomer. In some instances, at least one arginine residue is a D-form stereoisomer, and at least one arginine residue is a L-form stereoisomer. In some embodiments, the polyarginine tract comprises all D-form residues. In some embodiments, the polyarginine tract comprises all L-form residues. In some embodiments, the polyarginine tract comprises a combination of both D-form and L-form residues. In some embodiments, the polyarginine tract comprises only arginine residues. In some instances, the polyarginine tract is interrupted by at least one nonpolar residue. In some instances, the nonpolar residue is alanine. In certain instances, the alanine is D-alanine.

In one embodiment, the polyarginine tract of the peptide comprises the polyarginine tract, or derivative thereof, of any one of SEQ ID NOs: 1-40. In one embodiment, the polyarginine tract of the peptide comprises the polyarginine tract, or derivative thereof, of any one of SEQ ID NOs: 2-40. In one embodiment, the polyarginine tract, or derivative thereof, of the peptide comprises residues 27-35 of any one of SEQ ID NOs: 2-40.

In some embodiments, the peptide is conjugated to a label. In one embodiment, the C-terminal residue is such that the peptide is amenable to labeling. In one embodiment, the C-terminal residue is a lysine residue. In one embodiment, the label allows for purification of the peptide. In one embodiment, the label allows for detection of the peptide. In one embodiment, the label is biotin.

In one aspect, the present invention relates to a composition comprising a dual peptide system for targeting and delivery of an agent to a target cell. In one embodiment, the dual peptide system comprises (1) a first peptide that exhibits endosome-disruptive activity for delivery of an agent, as described herein, (2) a second peptide that exhibits a targeting function, and (3) an agent of interest.

In one embodiment, the second peptide that exhibits a targeting function comprises a targeting moiety. The targeting moiety of the second peptide can be any molecule that is capable of specifically binding or interacting with a desired target. In one embodiment, the targeting moiety can be any moiety recognized by a transmembrane or intracellular receptor protein. In one embodiment, a targeting moiety is a ligand. A ligand is usually a member of a binding pair where the second member is present on, or in a target cell, or in a tissue comprising the target cell. In one embodiment, targeting moiety is a ligand that binds to a tyrosine kinase receptor. In one embodiment, targeting moiety binds to epidermal growth factor receptor (EGFR). EGFR is often overexpressed in many types of cancer, including but not limited to, oral cancer.

In one embodiment, the second peptide is a derivative of GE11 peptide (Li, et al. FASEB J, 2005. 19:1978-1985). In one embodiment, the derivative of the GE11 peptide comprises a stretch of densely packed cationic amino acids residues. The stretch of cationic amino acid residues enables binding of nucleic acids to the peptide via electrostatic interactions. In one embodiment, the second peptide is GE11R9 (YHWYGYTPQNVIGGGGRRRRRRRRRK; SEQ ID NO: 41). GE11R9 maintains the EGFR targeting ability of GE11, while also containing a stretch of densely packed cationic amino acids to bind to nucleic acids (Alexander-Bryant, et al. Oral Oncol, 2017. 72: 123-131).

In one embodiment, the target moiety of the second peptide can be engineered to bind to a tumor antigen, thereby permitting specific targeting of a cancerous tumor expressing said tumor antigen. Tumor antigens are proteins that are produced by tumor cells that elicit an immune response; particularly T-cell mediated immune responses. The selection of the antigen binding moiety of the invention will depend on the particular type of cancer to be targeted or treated.

In some embodiments, the peptides of the present invention are labelled with a detectable marker. A wide range of detectable markers can be used, including but not limited to biotin, a fluorogen, an enzyme, an epitope, a chromogen, or a radionuclide. In one embodiment, the detectable marker can be conjugated to the C-terminal lysine of the peptide.

In certain embodiments, the peptides of the present invention further comprise conservative variants of the peptides herein described. As used herein, a “conservative variant” refers to alterations in the amino acid sequence that do not substantially and adversely affect the binding or association capacity of the peptide. A substitution, insertion or deletion is said to adversely affect the peptide when the altered sequence prevents, reduces, or disrupts a function or activity associated with the peptide. For example, the overall charge, structure or hydrophobic-hydrophilic properties of the peptide can be altered without adversely affecting an activity. Accordingly, the amino acid sequence can be altered, for example to render the peptide more hydrophobic or hydrophilic, without adversely affecting the activities of the peptide.

These variants, though possessing a slightly different amino acid sequence than those recited elsewhere herein, will still have the same or similar properties associated with any of the peptides discussed herein. Ordinarily, the conservative substitution variants, will have an amino acid sequence having at least 75%, at least 80%, at least 85%, at least 90%, at least 95%, at least 98%, or at least 99% sequence identity with any of the peptides discussed elsewhere herein.

With respect to the stretch of densely packed cationic amino acid residues in the peptides of the invention, the peptides can comprise at least 4 cationic amino acid residues, such as arginine. In other embodiments, the peptides can comprise at least 5 cationic amino acid residues, such as arginine. In other embodiments, the peptides can comprise at least 6 cationic amino acid residues, such as arginine. In other embodiments, the peptides can comprise at least 7 cationic amino acid residues, such as arginine. In other embodiments, the peptides can comprise at least 8 cationic amino acid residues, such as arginine. In other embodiments, the peptides can comprise at least 9 cationic amino acid residues, such as arginine. In other embodiments, the peptides may have a C-terminal lysine residue after the cationic amino acid residues. In further embodiments, the peptides may have a C-terminal lysine residue after the 9 cationic residues. The stretch of densely packed cationic amino acid residues is to enable binding of a nucleic acid to the peptide via electrostatic interactions.

In some embodiments, one or more components of the peptide delivery system of the invention are able to associate with (or bind to) nucleic acids, other proteins or small molecules. These peptides may be able to bind, for example, to nucleic acid molecules, other proteins, or small molecules with high affinity and selectivity. It is contemplated that such association may be mediated through specific sites on each of two (or more) interacting molecular species. Binding can be mediated by structural and/or energetic components. In some cases, the latter will comprise the interaction of molecules with opposite charges.

The peptides of the present invention may be made using chemical methods. For example, peptides can be synthesized by solid phase techniques (Roberge J Y et al (1995) Science 269: 202-204), cleaved from the resin, and purified by preparative high performance liquid chromatography. Automated synthesis may be achieved, for example, using the ABI 431 A Peptide Synthesizer (Perkin Elmer) in accordance with the instructions provided by the manufacturer.

The peptides may alternatively be made by recombinant means or by cleavage from a longer polypeptide. The composition of a peptide may be confirmed by amino acid analysis or sequencing.

The variants of the polypeptides according to the present invention may be (i) one in which one or more of the amino acid residues are substituted with a conserved or non-conserved amino acid residue (for example, a conserved amino acid residue) and such substituted amino acid residue may or may not be one encoded by the genetic code, (ii) one in which there are one or more modified amino acid residues, e.g., residues that are modified by the attachment of substituent groups, (iii) one in which the polypeptide is an alternative splice variant of the polypeptide of the present invention, (iv) fragments of the polypeptides and/or (v) one in which the polypeptide is fused with another polypeptide, such as a leader or secretory sequence or a sequence which is employed for purification (for example, His-tag) or for detection (for example, Sv5 epitope tag). The fragments include polypeptides generated via proteolytic cleavage (including multi-site proteolysis) of an original sequence. Variants may be post-translationally, or chemically modified. Such variants are deemed to be within the scope of those skilled in the art from the teaching herein.

### Peptide Analogs

In one embodiment, the present invention relates to peptide analogs of the peptides of the invention (e.g. 599 peptide or derivatives thereof) appropriate for use with the invention and uses thereof. For example, in certain instances the invention provides peptides and peptide analogs based on fragments of SEQ ID NO: 1-40 wherein the peptides, including peptides and analogs, fragments, and derivatives thereof, of the invention exhibit desirable properties. In some embodiments, the invention provides compositions comprising peptides and analogs, fragments, and derivatives thereof that exhibit one or more of improved solubility, half-life, bioavailability, reduced renal clearance and the like compared to full length 599 and its derivatives.

### Fusion, Chimeric and Modified Polypeptides

A peptide of the invention may be conjugated with other molecules, such as proteins, to prepare fusion proteins or chimeric peptides. This may be accomplished, for example, by the synthesis of N-terminal or C-terminal fusion proteins provided that the resulting fusion protein or chimeric peptide retains the functionality of the peptide of the invention.

In some embodiments, the peptide or chimeric protein of the present invention may be conjugated to a nuclear localization signal (NLS). A skilled artisan would recognize that any NLS known in the art capable of localizing the peptide or chimeric protein to the nucleus would be useful in the methods of the present invention.

A peptide or chimeric protein of the invention may be phosphorylated using conventional methods such as the method described in Reedijk et al. (The EMBO Journal 11(4):1365, 1992).

Cyclic derivatives of the peptides or chimeric proteins of the invention are also part of the present invention. Cyclization may allow the peptide or chimeric protein to assume a more favorable conformation for association with other molecules. Cyclization may be achieved using techniques known in the art. For example, disulfide bonds may be formed between two appropriately spaced components having free sulfhydryl groups, or an amide bond may be formed between an amino group of one component and a carboxyl group of another component. Cyclization may also be achieved using an azobenzene-containing amino acid as described by Ulysse, L., et al., J. Am. Chem. Soc. 1995, 117, 8466-8467. The components that form the bonds may be N-termini of the peptide, C-termini of the peptide, side chains of amino acids within the peptide, non-amino acid components, or any combination thereof. In an embodiment of the invention, cyclic peptides may comprise a beta-turn in the right position. Beta-turns may be introduced into the peptides of the invention by adding the amino acids Pro-Gly at the right position.

It may be desirable to produce a cyclic peptide which is more flexible than the cyclic peptides containing peptide bond linkages as described above. A more flexible peptide may be prepared by introducing cysteines at the right and left position of the peptide and forming a disulphide bridge between the two cysteines. The two cysteines are arranged so as not to deform the beta-sheet and turn. The peptide is more flexible as a result of the length of the disulfide linkage and the smaller number of hydrogen bonds in the beta-sheet portion. The relative flexibility of a cyclic peptide can be determined by molecular dynamics simulations.

In some embodiments, the subject compositions are peptidomimetics of the peptides of the invention. Peptidomimetics are compounds based on, or derived from, peptides and proteins. The peptidomimetics of the present invention typically can be obtained by structural modification of a known peptide sequence using unnatural amino acids, conformational restraints, isosteric replacement, and the like. The subject peptidomimetics constitute the continuum of structural space between peptides and non-peptide synthetic structures; peptidomimetics may be useful, therefore, in delineating pharmacophores and in helping to translate peptides into nonpeptide compounds with the activity of the parent peptides.

The peptidomimetics of the invention may include unnatural amino acids formed by post-translational modification or by introducing unnatural amino acids during translation. A variety of approaches are available for introducing unnatural amino acids during protein translation. By way of example, special tRNAs, such as tRNAs which have suppressor properties, suppressor tRNAs, have been used in the process of site-directed non-native amino acid replacement (SNAAR). In SNAAR, a unique codon is required on the mRNA and the suppressor tRNA, acting to target a non-native amino acid to a unique site during the protein synthesis (described in WO90/05785). However, the suppressor tRNA must not be recognizable by the aminoacyl tRNA synthetases present in the protein translation system. In certain cases, a non-native amino acid can be formed after the tRNA molecule is aminoacylated using chemical reactions which specifically modify the native amino acid and do not significantly alter the functional activity of the aminoacylated tRNA. These reactions are referred to as post-aminoacylation modifications. For example, the epsilon-amino group of the lysine linked to its cognate tRNA (tRNALYS), could be modified with an amine specific photoaffinity label.

In some embodiments, the peptides of the invention may be modified to produce variants, analogs and fragments of the proteins which have a reduced rate of clearance or increased half-life, so long as the desired biological properties (i.e., the ability to bind to the target site or the ability to have the same effector activity as the parent unmodified peptide) are retained. The peptides may be modified by using various genetic engineering or protein engineering techniques.

In one embodiment, a peptide of the invention is further modified. In one embodiment, a functional fragment of a peptide of the invention contains a further modification. In one example, a further modification of a peptide includes a modification at the N-terminus. In one embodiment a further modification comprises a modification at the C-terminus. In one embodiment a peptide of the invention is modified at both the N- and C-termini.

In one embodiment, a modification of a peptide of the invention is a chemical modification, conjugation to a synthetic or natural polymer, glycosylation, acetylation, methylation, phosphorylation, conjugation of a chemical linker, fatty acyl derivatization, or conjugation of a polysaccharide. In one embodiment, modification of a peptide of the invention includes fusion to human serum albumin (HAS) or an albumin-binding peptide or protein. In one embodiment, modification of a peptide of the invention includes linkage to an immunoglobin Fc domain to form an Fc fusion protein. In one embodiment, a polymer to be conjugated to a peptide on the invention is one of polyethylene glycol (PEG), polypropylene glycol (PPG), polysialic acid (PSA), XTEN™ and hydroxyethyl starch (HES). In one exemplary embodiment, PEGylation of one or more peptides of the invention can increase its half-life by retarding renal clearance, as the PEG moiety adds hydrodynamic radius to the peptide. Conventional PEGylation methodologies directed to monomeric proteins are well known in the art.

In one embodiment, a single polymer is conjugated to a single peptide of the invention. In one embodiment, multiple polymers are conjugated to a single peptide of the invention. In one embodiment, a population of peptides of the invention will contain a mixture of modified and unmodified peptides having 0 to more than 1 polymer conjugated to each peptide.

The peptides of the invention can be post-translationally modified. For example, post-translational modifications that fall within the scope of the present invention include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

### Nucleic Acid

The present invention further provides, in another embodiment, nucleic acid molecules that encode any of the amino acid sequences described herein. Those of ordinary skill in the art, given an amino acid sequence, will be able to generate corresponding nucleic acid sequences that can be used to generate the amino acid sequence, using no more than routine skill.

Modifications to the primary structure itself by deletion, addition, or alteration of the amino acids incorporated into the peptide sequence during translation can be made without destroying the activity of the peptide. Such substitutions or other alterations result in peptides having an amino acid sequence encoded by a nucleic acid falling within the contemplated scope of the present invention.

The present invention further provides, in some embodiments, recombinant nucleic acid molecules that contain a coding sequence. As used herein, a “recombinant DNA molecule” is a DNA molecule that has been subjected to molecular manipulation. Methods for generating recombinant DNA molecules are well known in the art, for example, see Sambrook et al., 2012, Molecular Cloning: A Laboratory Manual, Cold Spring Harbor Laboratory, New York. In some recombinant DNA molecules, a coding DNA sequence is operably linked to expression control sequences and vector sequences.

The choice of vector and expression control sequences to which one of the peptide family encoding sequences of the present invention is operably linked depends directly, as is well known in the art, on the functional properties desired (e.g., protein expression, and the host cell to be transformed). A vector of the present invention may be at least capable of directing the replication or insertion into the host chromosome, and also expression, of the structural gene included in the recombinant DNA molecule.

Expression control elements that are used for regulating the expression of an operably linked protein encoding sequence are known in the art and include, but are not limited to, inducible promoters, constitutive promoters, secretion signals, and other regulatory elements. In some embodiments, the inducible promoter is readily controlled, such as being responsive to a nutrient in the host cell's medium.

In one embodiment, the vector containing a coding nucleic acid molecule will include a prokaryotic replicon, i.e., a DNA sequence having the ability to direct autonomous replication and maintenance of the recombinant DNA molecule extra-chromosomal in a prokaryotic host cell, such as a bacterial host cell, transformed therewith. Such replicons are well known in the art. In addition, vectors that include a prokaryotic replicon may also include a gene whose expression confers a detectable marker such as a drug resistance. Typical of bacterial drug resistance genes are those that confer resistance to ampicillin or tetracycline.

Vectors that include a prokaryotic replicon can further include a prokaryotic or bacteriophage promoter capable of directing the expression (transcription and translation) of the coding gene sequences in a bacterial host cell, such as E. coli. A promoter is an expression control element formed by a DNA sequence that permits binding of RNA polymerase and transcription to occur. Promoter sequences compatible with bacterial hosts are typically provided in plasmid vectors containing convenient restriction sites for insertion of a DNA segment of the present invention. Any suitable prokaryotic host can be used to express a recombinant DNA molecule encoding a peptide of the invention.

Expression vectors compatible with eukaryotic cells, including those compatible with vertebrate cells, can also be used to form recombinant DNA molecules that contain a coding sequence. Eukaryotic cell expression vectors are well known in the art and are available from several commercial sources. Typically, such vectors are provided containing convenient restriction sites for insertion of the desired DNA segment.

Eukaryotic cell expression vectors used to construct the recombinant DNA molecules of the present invention may further include a selectable marker that is effective in a eukaryotic cell, such as a drug resistance selection marker. An example drug resistance marker is the gene whose expression results in neomycin resistance, i.e., the neomycin phosphotransferase (neo) gene. Alternatively, the selectable marker can be present on a separate plasmid, the two vectors introduced by co-transfection of the host cell, and transfectants selected by culturing in the appropriate drug for the selectable marker.

The present invention further provides, in yet another embodiment, host cells transformed with a nucleic acid molecule that encodes a peptide of the present invention. The host cell can be either prokaryotic or eukaryotic. Eukaryotic cells useful for expression of a peptide of the invention are not limited, so long as the cell line is compatible with cell culture methods and compatible with the propagation of the expression vector and expression of the gene product.

Transformation of appropriate cell hosts with a recombinant DNA molecule encoding a peptide of the present invention is accomplished by well-known methods that typically depend on the type of vector used and host system employed. With regard to transformation of prokaryotic host cells, electroporation and salt treatment methods can be employed (see, for example, Sambrook et al., 2012, Molecular Cloning: A Laboratory Manual, Cold Spring Harbor Laboratory Press). With regard to transformation of vertebrate cells with vectors containing recombinant DNA, electroporation, cationic lipid, or salt treatment methods can be employed (see, for example, Graham et al., (1973) Virology 52, 456-467; Wigler et al., (1979) Proc. Natl. Acad. Sci. USA 76, 1373-1376).

Successfully transformed cells can be identified by well-known techniques including the selection for a selectable marker. For example, cells resulting from the introduction of a recombinant DNA of the present invention can be cloned to produce single colonies. Cells from those colonies can be harvested, lysed and their DNA content examined for the presence of the recombinant DNA using a method such as that described by Southern (1975) J. MoI. Biol. 98, 503-517, or the peptides produced from the cell assayed via an immunological method.

The present invention further provides, in still another embodiment, methods for producing a peptide of the invention using nucleic acid molecules herein described. In general terms, the production of a recombinant form of a peptide typically involves the following steps: a nucleic acid molecule is obtained that encodes a peptide of the invention.

The nucleic acid molecule may then be placed in operable linkage with suitable control sequences, as described above, to form an expression unit containing the peptide open reading frame. The expression unit is used to transform a suitable host and the transformed host is cultured under conditions that allow the production of the recombinant peptide. Optionally the recombinant peptide is isolated from the medium or from the cells; recovery and purification of the peptide may not be necessary in some instances where some impurities may be tolerated.

Each of the foregoing steps can be done in a variety of ways. The construction of expression vectors that are operable in a variety of hosts is accomplished using appropriate replicons and control sequences, as set forth above. The control sequences, expression vectors, and transformation methods are dependent on the type of host cell used to express the gene. Suitable restriction sites, if not normally available, can be added to the ends of the coding sequence, so as to provide an excisable gene to insert into these vectors. An artisan of ordinary skill in the art can readily adapt any host/expression system known in the art for use with the nucleic acid molecules of the invention to produce a recombinant peptide.

### Bioactive Agent

In certain embodiments, the composition of the present invention comprises one or more agent which is delivered to a cell or tissue of interest via a peptide or peptides elsewhere herein. In certain embodiments, the agent interacts with the stretch of densely packed cationic amino acid residues of the peptide of the present invention. In other embodiments, the cellular uptake is enhanced by interrupting the densely packed stretch of cationic amino acid residues with a nonpolar residue, for example, alanine.

In some embodiments, the agent is a bioactive agent, wherein the agent exhibits a biological function or activity. Exemplary bioactive agents include, but are not limited to, nucleic acid molecules, proteins, peptides, antibodies, antibody fragments, and small molecules.

In one embodiment, the bioactive agent comprises a nucleic acid molecule selected from the group consisting of: siRNAs, microRNAs, shRNAs, antisense nucleic acids, ribozymes, killer-tRNAs, guide RNAs (part of the CRISPR/CAS system), long non-coding RNAs, anti-miRNA oligonucleotides, mRNAs, aptamers, and plasmid DNA.

In some embodiments, the nucleic acid molecule inhibits or suppresses the gene expression of a gene of interest. In some embodiments, the gene to be suppressed is an oncogene involved in cancer pathogenesis. For example, in certain embodiments the gene to be suppressed is CIP2A or HPV E6.

RNA interference (RNAi) is normally triggered by double-stranded RNA (dsRNA) or endogenous microRNA precursors (pri-miRNAs/pre-miRNAs). Since its discovery, RNAi has emerged as a powerful genetic tool for suppressing gene expression in mammalian cells. Stable gene knockdown can be achieved by expression of synthetic short hairpin RNAs (shRNAs).

An siRNA polynucleotide is an RNA nucleic acid molecule that interferes with RNA activity that is generally considered to occur via a post-transcriptional gene silencing mechanism. In one embodiment, the siRNA polynucleotide comprises a double-stranded RNA (dsRNA), but is not intended to be so limited and may comprise a single-stranded RNA (see, e.g., Martinez et al., 2002 Cell 110:563-74). The siRNA polynucleotide included in the invention may comprise other naturally occurring, recombinant, or synthetic single-stranded or double-stranded polymers of nucleotides (ribonucleotides or deoxyribonucleotides or a combination of both) and/or nucleotide analogues as provided herein (e.g., an oligonucleotide or polynucleotide or the like, typically in 5′ to 3′ phosphodiester linkage). Accordingly, it will be appreciated that certain exemplary sequences disclosed herein as DNA sequences capable of directing the transcription of the siRNA polynucleotides are also intended to describe the corresponding RNA sequences and their complements, given the well-established principles of complementary nucleotide base-pairings.

In one embodiment, siRNA polynucleotides comprise double-stranded polynucleotides of about 18-30 nucleotide base pairs. In another embodiment, siRNA polynucleotides comprise double-stranded polynucleotides of about 18, about 19, about 20, about 21, about 22, about 23, about 24, about 25, about 26, or about 27 base pairs, and in other embodiments about 19, about 20, about 21, about 22 or about 23 base pairs, or about 27 base pairs, whereby the use of “about” indicates that in certain embodiments and under certain conditions the processive cleavage steps that may give rise to functional siRNA polynucleotides that are capable of interfering with expression of a selected polypeptide may not be absolutely efficient. Hence, siRNA polynucleotides, may include one or more siRNA polynucleotide molecules that may differ (e.g., by nucleotide insertion or deletion) in length by one, two, three, four or more base pairs as a consequence of the variability in processing, in biosynthesis, or in artificial synthesis of the siRNA. The siRNA polynucleotide of the present invention may also comprise a polynucleotide sequence that exhibits variability by differing (e.g., by nucleotide substitution, including transition or transversion) at one, two, three or four nucleotides from a particular sequence. These differences can occur at any of the nucleotide positions of a particular siRNA polynucleotide sequence, depending on the length of the molecule, whether situated in a sense or in an antisense strand of the double-stranded polynucleotide. The nucleotide difference may be found on one strand of a double-stranded polynucleotide, where the complementary nucleotide with which the substitute nucleotide would typically form hydrogen bond base pairing, may not necessarily be correspondingly substituted. In some embodiments, the siRNA polynucleotides are homogeneous with respect to a specific nucleotide sequence.

Based on the present disclosure, it should be appreciated that the siRNAs of the present invention may effect silencing of the target polypeptide expression to different degrees. The siRNAs, thus, must first be tested for their effectiveness. Selection of siRNAs are made therefrom based on the ability of a given siRNA to interfere with or modulate the expression of the target polypeptide. Accordingly, identification of specific siRNA polynucleotide sequences that are capable of interfering with expression of a desired target polypeptide requires production and testing of each siRNA. The methods for testing each siRNA and selection of suitable siRNAs for use in the present invention are fully set forth herein the Examples. Since not all siRNAs that interfere with protein expression will have a physiologically important effect, the present disclosure also sets forth various physiologically relevant assays for determining whether the levels of interference with target protein expression using the siRNAs of the invention have clinically relevant significance.

In one embodiment, the agent is an siRNA directed against CIP2A. In one embodiment, the present invention provides a composition comprising a (a) peptide comprising a 599 variant, as described herein, and (b) an siRNA directed against CIP2A; thereby inhibiting CIP2A expression or activity in the subject.

In one embodiment, the agent is an siRNA directed against HPV E6. In one embodiment, the present invention provides a composition comprising a (a) peptide comprising the 599 peptide or a 599 variant, as described herein, and (b) an siRNA directed against HPV E6; thereby inhibiting HPV E6 expression or activity in the subject.

In one embodiment, the agents are separate siRNAs directed against CIP2A and HPV E6. In one embodiment, the present invention provides a composition comprising a (a) peptide or peptides comprising the 599 peptide or a 599 variant, as described herein, and (b) an siRNA directed against CIP2A and an siRNA directed against HPV E6; thereby inhibiting CIP2A and HPV E6 expression or activity in the subject.

One skilled in the art will readily appreciate that as a result of the degeneracy of the genetic code, many different nucleotide sequences may encode the same polypeptide. That is, an amino acid may be encoded by one of several different codons, and a person skilled in the art can readily determine that while one particular nucleotide sequence may differ from another, the polynucleotides may in fact encode polypeptides with identical amino acid sequences. As such, polynucleotides that vary due to differences in codon usage are specifically contemplated by the present invention.

In one embodiment, the modulating sequence is an antisense nucleic acid sequence which is expressed by a plasmid vector. The antisense expressing vector is used to transfect a mammalian cell or the mammal itself, thereby causing reduced endogenous expression of a desired regulator in the cell. However, the invention should not be construed to be limited to inhibiting expression of a regulator by transfection of cells with antisense molecules. Rather, the invention encompasses other methods known in the art for inhibiting expression or activity of a protein in the cell including, but not limited to, the use of a ribozyme, the expression of a non-functional regulator (i.e. transdominant negative mutant) and use of an intracellular antibody.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the invention may be made synthetically and then provided to the cell. Antisense oligomers of between about 10 to about 30, and in some instances about 15 nucleotides, are easily synthesized and introduced into a target cell. Synthetic antisense molecules contemplated by the invention include oligonucleotide derivatives known in the art which have improved biological activity compared to unmodified oligonucleotides (see U.S. Pat. No. 5,023,243).

Ribozymes and their use for inhibiting gene expression are also well known in the art (see, e.g., Cech et al., 1992, J. Biol. Chem. 267:17479-17482; Hampel et al., 1989, Biochemistry 28:4929-4933; Eckstein et al., International Publication No. WO 92/07065; Altman et al., U.S. Pat. No. 5,168,053). Ribozymes are RNA molecules possessing the ability to specifically cleave other single-stranded RNA in a manner analogous to DNA restriction endonucleases. Through the modification of nucleotide sequences encoding these RNAs, molecules can be engineered to recognize specific nucleotide sequences in an RNA molecule and cleave it (Cech, 1988, J. Amer. Med. Assn. 260:3030). A major advantage of this approach is the fact that ribozymes are sequence-specific.

There are two basic types of ribozymes, namely, tetrahymena-type (Hasselhoff, 1988, Nature 334:585) and hammerhead-type. Tetrahymena-type ribozymes recognize sequences which are four bases in length, while hammerhead-type ribozymes recognize base sequences 11-18 bases in length. The longer the sequence, the greater the likelihood that the sequence will occur exclusively in the target mRNA species. Consequently, hammerhead-type ribozymes are preferable to tetrahymena-type ribozymes for inactivating specific mRNA species, and 18-base recognition sequences are preferable to shorter recognition sequences, which may occur randomly within various unrelated mRNA molecules.

Ribozymes useful for inhibiting the expression of a regulator may be designed by incorporating target sequences into the basic ribozyme structure, which are complementary to the mRNA sequence of the desired target of the present invention. Ribozymes targeting the desired regulator may be synthesized using commercially available reagents (Applied Biosystems, Inc., Foster City, CA) or they may be genetically expressed from DNA encoding them.

In some embodiments of the invention, a miRNA or a synthetic miRNA is used as a therapeutic agent to regulate gene expression. The miRNA may contain one or more design elements. These design elements include, but are not limited to: i) a replacement group for the phosphate or hydroxyl of the nucleotide at the 5′ terminus of the complementary region; ii) one or more sugar modifications in the first or last 1 to 6 residues of the complementary region; or, iii) noncomplementarity between one or more nucleotides in the last 1 to 5 residues at the 3′ end of the complementary region and the corresponding nucleotides of the miRNA region.

In certain embodiments, a synthetic miRNA has a nucleotide at its 5′ end of the complementary region in which the phosphate and/or hydroxyl group has been replaced with another chemical group (referred to as the “replacement design”). In some cases, the phosphate group is replaced, while in others, the hydroxyl group has been replaced. In particular embodiments, the replacement group is biotin, an amine group, a lower alkylamine group, an acetyl group, 2′O-Me (2′ oxygen-methyl), DMTO (4,4′-dimethoxytrityl with oxygen), fluoroscein, a thiol, or acridine, though other replacement groups are well known to those of skill in the art and can be used as well.

Additional embodiments concern a synthetic miRNA having one or more sugar modifications in the first or last 1 to 6 residues of the complementary region (referred to as the “sugar replacement design”). In certain cases, there are one or more sugar modifications in the first 1, 2, 3, 4, 5, 6 or more residues of the complementary region, or any range derivable therein. In additional cases, there are one or more sugar modifications in the last 1, 2, 3, 4, 5, 6 or more residues of the complementary region, or any range derivable therein, that have a sugar modification. It will be understood that the terms “first” and “last” are with respect to the order of residues from the 5′ end to the 3′ end of the region. In particular embodiments, the sugar modification is a 2′O-Me modification. In further embodiments, there are one or more sugar modifications in the first or last 2 to 4 residues of the complementary region or the first or last 4 to 6 residues of the complementary region.

In other embodiments of the invention, there is a synthetic miRNA in which one or more nucleotides in the last 1 to 5 residues at the 3′ end of the complementary region are not complementary to the corresponding nucleotides of the miRNA region (“noncomplementarity”) (referred to as the “noncomplementarity design”). The noncomplementarity may be in the last 1, 2, 3, 4, and/or 5 residues of the complementary miRNA. In certain embodiments, there is noncomplementarity with at least 2 nucleotides in the complementary region.

The miRNA region and the complementary region may be on the same or separate polynucleotides. In cases in which they are contained on or in the same polynucleotide, the miRNA molecule will be considered a single polynucleotide. In embodiments in which the different regions are on separate polynucleotides, the synthetic miRNA will be considered to be comprised of two polynucleotides.

When the RNA molecule is a single polynucleotide, there is a linker region between the miRNA region and the complementary region. In some embodiments, the single polynucleotide is capable of forming a hairpin loop structure as a result of bonding between the miRNA region and the complementary region. The linker constitutes the hairpin loop. It is contemplated that in some embodiments, the linker region is, is at least, or is at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or40 residues in length, or any range derivable therein. In certain embodiments, the linker is between 3 and 30 residues (inclusive) in length.

In addition to having a miRNA region and a complementary region, there may be flanking sequences as well at either the 5′ or 3′ end of the region. In some embodiments, there is or is at least 1, 2, 3, 4, 5, 6, 7, 8, 9, 10 nucleotides or more, or any range derivable therein, flanking one or both sides of these regions.

In one embodiment, the nucleic acid molecule is a DNA, RNA, cDNA molecule, or the like which encodes a therapeutic peptide or protein. For example, in certain embodiments, once the nucleic acid molecule enters a desired cell, via one or more of the peptides described herein, the nucleic acid molecule may be transcribed and/or translated into a desired therapeutic peptide.

### Methods

In one embodiment, the present invention is a method for delivering one or more agent to a subject or a cell. In one embodiment, the method comprises contacting a subject or a cell with a composition comprising one or more peptide described herein (e.g., 599 peptide or derivatives thereof) and one or more agent.

In one embodiment, the method comprises contacting a subject or cell with a composition comprising one or more first peptide with endosome-disrupting capabilities (e.g. 599 peptide or derivatives thereof), one or more second peptide with targeting abilities (e.g. GE11R9), and one or more agent.

In some embodiments, the present invention provides methods for transporting a desired agent into a variety of mammalian, amphibian, reptilian, avian, or insect cells. Cells can be primary cells or cell lines. Mammalian cells include, but are not limited to, human, monkey, rat, mouse, dog, cow, pig, horse, hamster, and rabbit. Primary cells from mammals include, but are not limited to, adipocytes, astrocytes, cardiac muscle cells, chondrocytes, endothelial cells, epithelial cells, fibroblasts, gangliocytes, glandular cells, glial cells, hematopoietic cells, hepatocytes, keratinocytes, myoblasts, neural cells, osteoblasts, ovary cells, pancreatic beta cells, renal cells, smooth muscle cells, and striated muscle cells.

In various embodiments, the invention provides methods of treating a disease or disorder in a subject in need thereof. In one embodiment, the method comprises contacting a subject having a disease or disorder with a composition comprising one or more peptides described herein (e.g., 599 peptide or derivatives thereof) and one or more agent. In one embodiment, the method comprises contacting a subject having a disease or disorder with a composition comprising one or more peptides described herein (e.g., 599 peptide or derivatives thereof) and one or more bioactive agent; wherein the bioactive agent exhibits a biological function or activity to reduce or ameliorate one or more signs or symptoms of the disease or disorder.

In one embodiment, the method comprises contacting a subject having a disease or disorder with a composition comprising one or more first peptide with endosome-disrupting capabilities (e.g. 599 peptide or derivatives thereof), one or more second peptide with targeting abilities (e.g. GE11R9), and one or more agent; wherein the agent exhibits a biological function or activity to reduce or ameliorate one or more signs or symptoms of the disease or disorder.

In some embodiments of the invention, peptides of the invention are administered to a subject in an effective amount to treat, prevent, or otherwise alleviate symptoms of a cancer, disease or disorder. The peptides of the invention may be administered to cells of a subject to treat, prevent, or otherwise alleviate symptoms of a disease or disorder (e.g., cancers) alone or in combination with the administration of other therapeutic or bioactive compounds for the treatment or prevention of these diseases or disorders.

In one embodiment, the agent is an siRNA directed against CIP2A. In one embodiment, the present invention provides a method comprising administering to a subject a (a) peptide comprising a 599 variant, as described herein, and (b) an siRNA directed against CIP2A; thereby inhibiting CIP2A expression or activity in the subject.

In one embodiment, the agent is an siRNA directed against HPV E6. In one embodiment, the present invention provides a method comprising administering to a subject a (a) peptide comprising the 599 peptide or a 599 variant, as described herein, and (b) an siRNA directed against HPV E6; thereby inhibiting HPV E6 expression or activity in the subject.

In some embodiments, the present invention provides methods of treating or preventing cancer in a subject by reducing the expression or activity of CIP2A or HPV E6 in a subject, by administering to the subject a 599 peptide or 599 peptide variant, as described herein, in combination with an siRNA targeted against CIP2A or HPV E6. In some embodiments, the present invention provides methods of treating or preventing cancer in a subject by reducing the expression or activity of CIP2A and HPV E6 in a subject, by administering to the subject a 599 peptide or 599 peptide variant, as described herein, in combination with an siRNA targeted against CIP2A and an siRNA targeted against HPV E6.

In one embodiment, the peptides of the invention are administered with a desired agent at a molar ratio that exhibits effective binding and specific uptake of the agent into the cell. The molar ratio can be optimized in order to synergistically mediate the effective targeting and delivery of the agent into the cell and result in optimal targeting with minimal cytotoxicity.

In one embodiment, a 1:1 molar ratio of peptide and the agent is used. In certain aspects of the present invention, a molar ratio of peptide to agent is used that increases agent binding to the peptide, cellular uptake of the agent, activity of the agent, or release of the agent as compared to the effects observed using a molar ratio of 1:1. In one embodiment the molar ratio of peptide to agent ranges from 1:1 to 100:1 and all integer values there between. In certain embodiments the molar ratio of peptide to reagent is about 1:1, about 10:1, about 20:1, about 30:1, about 40:1, about 50:1, about 60:1, about 70:1, about 80:1 about 90:1 and about 100:1. In one embodiment the ratio of peptide to reagent is about 50:1.

In one embodiment, a 1:1:1 molar ratio of the second peptide that exhibits a targeting moiety (2^{nd} peptide), the first peptide that exhibits an endosome-disruptive bioactivity (1^{st} peptide), and the agent is used. In certain aspects of the present invention, a molar ratio of 2^{nd} peptide:1^{st} peptide:agent is used, such that an increase in targeting a cell, endosome-disruptive bioactivity, and activity of the agent is observed as compared to the effects observed using a ratio of 1:1:1. In one embodiment, the molar ratio of 2^{nd} peptide:1^{st} peptide:agent ranges from 100:1:1 to 1:100:1 and all integer values there between. In one aspect of the present invention, more of the 2^{nd} peptide is used compared to the 1^{st} peptide. In certain embodiments of the invention, the molar ratio of 2^{nd} peptide:1^{st} peptide:agent is about 100:10:1; about 100:20:1; about 100:30:1; about 100:40:1; about 100:50:1; about 100:60:1; about 100:70:1; about 100:80:1; about 100:90:1; about 100:100:1. In certain embodiments of the invention, the molar ratio of 2^{nd} peptide:1^{st} peptide:agent is about 10:100:1; about 20:100:1; about 30:100:1; about 40:100:1; about 50:100:1; about 60:100:1; about 70:100:1; about 80:100:1; about 90:100:1; about 100:100:1. In certain embodiments, the molar ratio of 2^{nd} peptide:1^{st} peptide:agent is about 60:30:1.

As those of ordinary skill in the art can readily appreciate, the molar ratios used may depend on the cell type. The molar ratios may also depend on the agent. One of skill in the art will appreciate that a variety of other molar ratios may be suitable for use in the present invention.

It will be appreciated that the peptides of the invention may be administered to a subject either alone, or in conjunction with another bioactive or therapeutic agent. In one embodiment, the peptides of the invention are administered to a subject in combination with an anti-cancer therapy.

### Pharmaceutical Compositions and Dosing

The therapeutic and prophylactic methods of the invention thus encompass the use of pharmaceutical compositions comprising one or more components of the peptide delivery system of the invention to practice the methods of the invention. The pharmaceutical compositions useful for practicing the invention may be administered to deliver a dose of from 100 ng/kg/day to 100 mg/kg/day. In one embodiment, the invention envisions administration of a dose which results in a concentration of the compound of the present invention from 1 μM to 10 μM in a mammal.

Typically, dosages which may be administered in a method of the invention to a mammal, a human for example, range in amount from 0.5 μg to about 50 mg per kilogram of body weight of the mammal. The precise dosage administered will vary depending upon any number of factors, including but not limited to, the type of mammal and type of disease state being treated, the age of the mammal and the route of administration. In one embodiment, the dosage of the compound will vary from about 1 μg to about 10 mg per kilogram of body weight of the mammal. In another embodiment, the dosage will vary from about 3 μg to about 1 mg per kilogram of body weight of the mammal.

The compound may be administered to a mammal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the mammal, etc.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions, which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals, including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, oral, rectal, vaginal, parenteral, topical, pulmonary, intranasal, buccal, or another route of administration. Other contemplated formulations include projected nanoparticles, liposomal preparations, resealed erythrocytes containing the active ingredient, and immunologically-based formulations.

The peptides of the invention, or any chimeric proteins or derivatives thereof, may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient, which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

A pharmaceutical composition of the invention may be prepared, packaged, or sold in a formulation suitable for buccal administration. Such formulations may, for example, be in the form of tablets or lozenges made using conventional methods, and may, for example, contain 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or atomized formulations, when dispersed, have an average particle or droplet size in the range from about 0.1 to about 200 nanometers, and may further comprise one or more of the additional ingredients described herein.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions, such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, PA), which is incorporated herein by reference

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Advancing Peptide siRNA-Carrier Designs for Delivery to Cancer Cells

To better understand the importance of stereochemistry in peptide carrier design/function for siRNA-based cellular delivery, eight 599 peptide variants were designed, incorporating either different stereochemical patterns of L/D-amino acids or a specific D-amino acid substitution, and then characterized in their ability to bind, deliver, stabilize, and release siRNAs, as well as induce effective gene silencing. The results demonstrate that incorporation of different stereochemical patterns of L/D-amino acids or a specific D-amino acid substitution within the 599 peptide design, could in some instances increase/decrease the binding, nuclease/serum stability, and complex release of siRNAs, as well as influence the gene silencing efficiencies of the complex, in comparison to the native 599 peptide. Moreover, these modifications in 599 peptide design were also found to affect cellular uptake and intracellular localization patterns of siRNA cargo to various degrees, with one particular 599 peptide variant that contained the specific D-amino acid substitution, capable of mediating a more ordered binding of siRNAs to specific cellular projections, identified as filopodia. Interestingly, this specific variant also exhibited the most enhanced gene silencing in comparison to the native 599 peptide, thus, implying that its peptide design modification could be responsible for directing a more efficient mode of siRNA drug delivery, resulting in the enhancement of gene silencing.

The materials and methods for this example are now described.

### Peptide Synthesis.

The peptides listed in Table 1 were synthesized by the solid-phase peptide synthesis process and purified (>95% purity) by high-performance liquid chromatography (HPLC) at GenScript (Piscataway, NJ). D-amino acids were used directly during the synthesis process and hydroxyl benzotriazole (HBOt) was added to suppress racemizations. All peptides were also biotinylated on the C-terminal lysine residue, similar to 599 (Cantini, et al. PLoS One, 2013. 8(9): e73348), which was originally added to the design to serve as a biological tag. Electrospray ionization mass spectrometry and HPLC analyses of the purified peptides did not reveal any instances of amino acid racemizations.

TABLE 1
The amino acid sequences of 599
peptide and its L/D-amino acid
stereochemically-modified variants.

| | | SEQ |
|---|---|---|
| Peptide | | ID |
| name | Peptide sequence^{a,b} | NO: |
| 599 | GLFEAIEGFIENGWEGMIDGWY | 1 |
| | GGGGrrrrrrrrrK-biotin | |
| RD3AD | GLFEAIEGFIENGWEGMIDGWY | 20 |
| | GGGGrrarrrrrrK-biotin | |
| RD456 | GLFEAIEGFIENGWEGMIDGWY | 2 |
| | GGGGRRRrrrRRRK-biotin | |
| All-L | GLFEAIEGFIENGWEGMIDGWY | 3 |
| | GGGGRRRRRRRRRK-biotin | |
| RD19 | GLFEAIEGFIENGWEGMIDGWY | 4 |
| | GGGGrRRRRRRRrK-biotin | |
| RD123789 | GLFEAIEGFIENGWEGMIDGWY | 5 |
| | GGGGrrrRRRrrrK-biotin | |
| RD13579 | GLFEAIEGFIENGWEGMIDGWY | 6 |
| | GGGGrRrRrRrRrK-biotin | |
| All-D | GlfeaieGfienGweGmidGwy | 7 |
| | GGGGrrrrrrrrrk-biotin | |
| INF7D | GlfeaieGfienGweGmidGwy | 8 |
| | GGGGRRRRRRRRRK-biotin | |

ªD-amino acids are indicated by a lower script letter.
bSubstituted D-arginine residue for D-alanine in RD3AD is underlined.

siRNAs.

siCIP2A, its DY547 fluorescently-labelled derivative, DY547-siCIP2A, and the negative control siGENOME™ Non-Targeting siRNA #5, siNT, were previously described (Cantini, et al. PLoS One, 2013. 8(9): e73348; Alexander-Bryant, et al. J Control Release, 2015. 218: 72-81; Alexander-Bryant, et al. Oral Oncol, 2017. 72: 123-131; Junttila, et al. Cell, 2007. 130(1): 51-62) and synthesized by Horizon Discovery Dharmacon™ (Lafayette, CO).

### Cell Culture.

Human tongue squamous cell carcinoma (SCC) cell lines CAL 27 and UPCI:SCC090 (SCC-90) were purchased from American Type Culture Collection (ATCC, Manassas, VA). The cell lines were cultured in ATCC-specified complete growth media in a 37° C. incubator with 5% CO₂. All experiments using these two cell lines were performed at passages less than 20.

siRNA Binding Assay.

30 pmol of siCIP2A was complexed with 599 peptide (or the peptide variants) at 1:1, 5:1, 10:1, 20:1, 30:1, 40:1 and 50:1 Peptide:siRNA molar ratios (equivalent to 0.25, 1.25, 2.5, 5, 7.5, 10, and 12.5 N/P ratios, respectively, with the exception of RD3AD, whose N/P ratios were instead 0.225, 1.125, 2.25, 4.5, 6.75, 9, and 11.25, respectively) at room temperature (RT) for 25 minutes. Afterwards, the samples were electrophoresed on a 4% agarose gel and stained with ethidium bromide. Free siCIP2A was used as a normalizing control. An Ultra-Low Range DNA Ladder (Thermo Fisher Scientific, Waltham, MA) was used as a MW marker. Resulting siCIP2A bands were imaged and quantified using a G:Box Chemi XX6 and GeneSys image capture software (Syngene, Frederick, MD), respectively.

Quantitative siRNA Cell Uptake Assay.

CAL 27 cells, grown to a confluency of 60% on a 24-well plate, were rinsed three times with Opti-MEM I Reduced Serum Media (Opti-MEM; Thermo Fisher Scientific). Meanwhile, 60 pmol of DY547-siCIP2A was incubated either alone or with the 599 peptide (or the 599 peptide variants) at 30:1 and 50:1 Peptide:siRNA molar ratios at RT for 25 minutes to allow for complexation. After complexation, the total volume was brought to 600 μL with Opti-MEM, after which the cells were incubated with 500 μL of peptide-siCIP2A complexes for 2 hours at 37° C. with 5% CO2. As an additional control, the cells were also transfected with 50 pmol of DY547-siCIP2A using Lipofectamine® 3000 (Thermo Fisher Scientific), according to the manufacturer's instructions. After treatment, the cells were rinsed three times with PBS, before being trypsinized for 10 minutes at 37° C., followed by centrifugation for 5 minutes at 1,000×g at 4° C. The cells were then washed with ice cold phosphate-buffered saline (PBS) and centrifuged again for 5 minutes at 1,000×g at 4° C. Next, the cells were lysed in 250 μl ice cold 0.1 M NaOH. The cell lysates were centrifuged at 4° C. for 5 minutes at 10,000×g to remove cell debris. Afterwards, 100 μL of each sample was transferred to a black 96-well plate to measure fluorescence at 530/590 nm using a BioTek (Winooski, VT) Synergy HT plate reader. Fluorescence measurements were converted to the amount of internalized siRNAs using a standard curve that was generated per experiment using known concentrations of DY547-siCIP2A ranging from 0-100 fmol/μl. Subsequently, the amount of internalized siRNA was normalized to the amount of protein, which was quantitated using the Pierce BCA Protein Assay Kit (Thermo Fisher Scientific).

Confocal Fluorescence Microscopy Analysis of Cell Uptake and Localization of siRNAs.

CAL 27 (or SCC-90) cells, grown to a confluency of 60% on BioCoat™ Collagen Type I coated 8-chamber slides (Corning, Corning, NY), were rinsed three times with Opti-MEM. Meanwhile, 20 pmol of DY547-siCIP2A was complexed with 599 peptide (or the peptide variants) at 50:1 Peptide:siRNA molar ratios for 25 minutes at RT in Opti-MEM. Afterwards, the complexes were added to the cells for incubation at 37° C. with 5% CO₂ for 2 hours. For the time course experiment, the cells were incubated with the specified complexes for 0.5, 1, 2, and 4 hours. Subsequently, the cells were rinsed with PBS, fixed in 4% paraformaldehyde at RT for 10 minutes, and then the slides were either mounted with coverslips using VECTASHIELD Mounting Medium with 4′,6-diamidino-2-phenylindole (DAPI, VECTOR Laboratories, Burlingame, CA) or permeabilized with 0.1% Triton X-100 at RT for 5 minutes and then incubated with Alexa Fluor™ 488 Phalloidin (Thermo Fisher Scientific) for 20 minutes. Afterwards, the slides containing the permeabilized cells were mounted with coverslips, as described above. Fluorescence images were obtained using a Zeiss (Thornwood, NY) 880 LSM NLO (with a Fast Airyscan super resolution detector) confocal microscope equipped with ×25 and ×63 objectives.

### Physicochemical Measurements:

The hydrodynamic diameter, zeta potential, and polydispersity index of 599 and its peptide variants in complex with siCIP2A formulated at a 50:1 Peptide:siRNA molar (12.5 N/P) ratio in water was measured using a Malvern Panalytical Zetasizer ZS instrument.

### Viability Assay.

Long-term cell viability was assessed using the CellTiter 96® AQueous One Solution Cell Proliferation Assay (Promega, Madison, WI). Briefly, CAL 27 cells were grown to 60% cellular confluency on 96-well plates, after which the cells were treated in Opti-MEM with 125 nM siNT in complex with 599 peptide (or the 599 peptide variants) at 50:1 Peptide:siRNA molar ratios for 4 hours, prior to adjusting the siRNA and fetal bovine serum (FBS) concentrations to 100 nM and 10%, respectively, by adding 50% FBS/Opti-MEM media. After 48 hours of treatment, the cell viability was assayed, according to the manufacturer's instructions. Absorbance at 490 nm was measured using a BioTek Synergy HT plate reader. Untreated cells were defined as 100% viable.

### RNase Protection Assay.

30 pmol of siCIP2A was complexed with 599 peptide (or the 599 peptide variants) at 50:1 Peptide:siRNA molar ratios at RT for 25 minutes. Afterwards, complexes were either treated or untreated with 0.18 μg of RNase A (Thermo Fisher Scientific) for 1 hour at 37° C., followed by denaturation with 4% SDS. Next, the samples were electrophoresed on a 4% agarose gel and resulting siCIP2A bands were imaged and quantified, as described above.

### Serum Protection Assay.

30 pmol of siCIP2A was complexed with 599 peptide (or the 599 peptide variants) at 50:1 Peptide:siRNA molar ratios at RT for 25 minutes. Afterwards, complexes were either treated or untreated with 50% human serum (final concentration; VWR, Radnor, PA) for 1 hour at 37° C., followed by denaturation with 4% SDS. Next, the samples were electrophoresed on a 4% agarose gel and resulting siCIP2A bands were imaged and quantified, as described above.

siRNA Release Assay.

30 pmol of siCIP2A was complexed with 599 peptide (or the 599 peptide variants) at 50:1 Peptide:siRNA molar ratios at RT for 25 minutes. Afterwards complexes were incubated with either 0, 0.1, 0.5, 1.0, 2.0, 5.0 or 10 μg heparin (Millipore Sigma, St. Louis, MO) for 30 minutes at RT. The samples were then electrophoresed on a 4% agarose gel and resulting siCIP2A bands were imaged and quantified, as described above.

### CIP2A Silencing in Cells.

CAL 27 cells, grown to a confluency of 60% on 24-well plates, were rinsed three times with Opti-MEM. Meanwhile, 60 pmol of either siNT or siCIP2A were complexed with 599 peptide (or the 599 peptide variants) at 50:1 Peptide:siRNA molar ratios for 25 minutes at RT in Opti-MEM. Afterwards, the complexes were added to the cells at a siRNA concentration of 125 nM and incubated for 4 hours at 37° C. with 5% CO₂, prior to adjusting the siRNA and FBS concentrations to 100 nM and 10%, respectively, with the addition of 50% FBS/Opti-MEM media. 48 hours post-treatment, the total RNA or protein was harvested for subsequent real-time PCR and Western blot analyses.

### Real-Time PCR.

Total RNA was harvested using the RNeasy Mini Kit (Qiagen, Germantown, MD). RNA was then reverse transcribed using the High-Capacity cDNA Reverse Transcription Kit (Thermo Fisher Scientific) in a Bio-Rad (Hercules, CA) T100 Thermal Cycler. Next, quantitative real-time PCR was performed in an Applied Biosystems™ StepOnePlus™ Real-Time PCR machine (Thermo Fisher Scientific) using the TaqMan™ Fast Advanced Master Mix (Thermo Fisher Scientific) and predesigned TaqMan™ Gene Expression Assays (Thermo Fisher Scientific) for CIP2A (Hs00405413_m1) and 18S (4319413E), according to the manufacturer's instructions.

### Western Blot Analysis.

Treated cells were washed twice with PBS and lysed using ice cold RIPA buffer (50 mM Tris-HCl, pH 7.4, 150 mM NaCl, 0.5% sodium deoxycholate, 0.1% SDS, and 1% NP-40) with protease inhibitor cocktail (Thermo Fisher Scientific). The protein lysates were quantified using the Pierce BCA Protein Assay Kit (Thermo Fisher Scientific) and then resolved by SDS-PAGE on a Mini-PROTEAN TGX stain free, 4-20% gradient gel (Bio-Rad). Afterwards, samples were transferred to a nitrocellulose membrane using the Bio-Rad Trans-Blot Turbo Transfer System. The nitrocellulose membrane was then cut into two pieces at ˜60-kDa, to generate an upper membrane (≥60-kDa) and lower membrane (<60-kDa), after which both were blocked in 5% (w/v) nonfat dried milk in Tris-HCl-buffered-saline—0.1% Tween (TBS-T; pH 7.5) for 1 hour at RT. The upper membrane was then incubated with mouse monoclonal anti-CIP2A antibody (1:500, clone 2G10-3B5, Santa Cruz Biotechnology, Santa Cruz, CA) and the lower membrane was incubated with mouse monoclonal anti-β-actin antibody (1:5000, clone AC-15, Millipore Sigma) in 5% (w/v) nonfat dried milk in TBS-T overnight at 4° C. Subsequently, the membranes were washed four times, five minutes each, with TBS-T and then incubated with horseradish peroxidase-conjugated-goat anti-mouse secondary antibody (1:3000 or 1:5000, SouthernBiotech, Birmingham, AL) in 5% (w/v) nonfat dried milk in TBS-T for 1 hour at RT. Immunoreactive bands were detected using Bio-Rad Clarity or Bio-Rad Clarity Max substrate systems, according to the manufacturer's instructions, with resulting images captured using a G:Box Chemi XX6 system (Syngene).

The results of this example are now described.

L/D-Amino Acid Stereochemical Modifications to the 599 Peptide Carrier can Either Increase or Decrease their Ability to Bind, Deliver, Protect, and Release siRNAs.

In advancing the 599 peptide carrier design for siRNA-based therapeutics, for the purposes of this Example, the focus was on the role of amino acid stereochemistry. Previously, it had been demonstrated that modulation of peptide stereochemistry could affect the cell uptake efficiency of both free arginine-rich-containing CPPs and that of antisense oligonucleotide-bound CPP polyplexes (Verdurmen, W P, et al., Chem Biol, 2011. 18(8): 1000-1010; Favretto, M E, and Brock, R, Small, 2015, 11: 1414-1417). Accordingly, to examine the effects of peptide stereochemistry on the delivery of siRNA cargo, seven 599 peptide variants were designed comprising different patterns of L/D-amino acids (see Table 1 above for peptide sequences), with several designs based on the polyarginine stereopeptide sequences used in the Verdurmen et al. study (Chem Biol, 2011. 18(8): 1000-1010.) Additionally, an eighth 599 peptide variant was also designed comprising a D-arginine substitution for D-alanine (RD3AD), based on evidence that a similar modification within a receptor-targeting CPP could lead to improved siRNA release from a multifunctional peptide complex (Jun, et al. PLoS One, 2015. 10(2): e0118310).

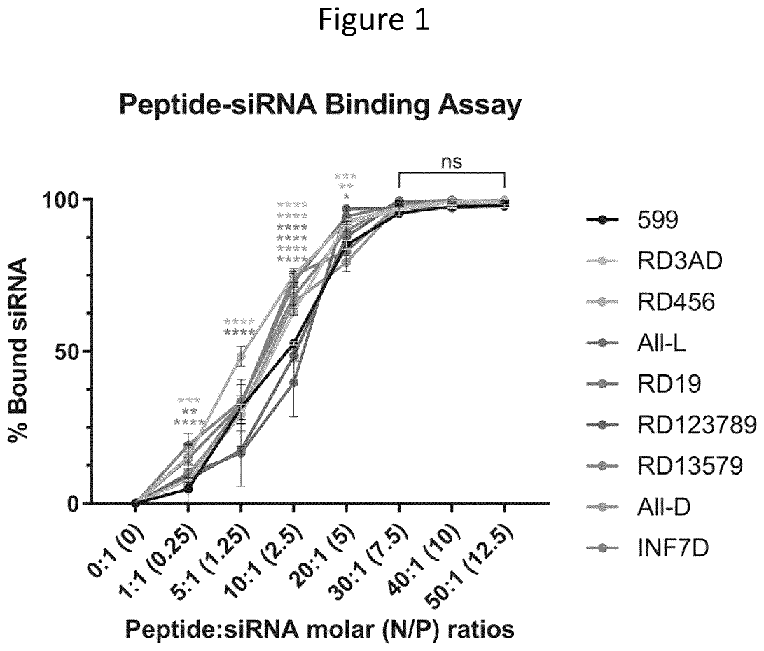

Upon chemically synthesizing the peptides, gel-shift assays were performed to determine first, if L/D-amino acid stereochemical modifications to the 599 peptide could alter its ability to bind free siRNAs through electrostatic interactions between the anionic siRNAs and the C-terminal cationic polyarginine sequence found within the peptide. Using siCIP2A, an siRNA designed to target the CIP2A oncogene, it was found that complexation of this siRNA with increasing amounts of 599 peptide or its peptide variants, ranging from 1 to 50-fold molar excess of the siRNA, could significantly affect the binding properties of the 599 peptide to siRNAs (FIG. 1). In particular, at peptide-to-siRNA (Peptide:siRNA) molar ratios of 1:1 through 20:1 (equivalent to nitrogen:phosphate (N/P) ratios of 0.25 through 5 for all peptides except RD3AD, whose N/P ratios were slightly less due to the D-arginine substitution for D-alanine; please see materials & methods section for the specific N/P ratio values for all peptides with respect to the Peptide:siRNA molar ratios), most 599 peptide variants were found to bind significantly better to the siRNA compared to the native 599 peptide, with peptides RD456 and RD19 showing the most consistent enhancement in binding. Conversely, peptides RD123789 and All-L (comprising only all L-amino acids), exhibited largely no significant changes in siRNA binding relative to 599. At Peptide:siRNA molar ratios of 30:1 (N/P ratios of 7.5) and greater, however, all peptide variants bound equally to ˜100% of the siRNAs, with no significant differences observed in comparison to 599. Thus, these data indicated that siRNA binding capacities to peptide carriers could be influenced by sequence-specific L/D-amino acid stereochemical changes, but only at Peptide:siRNA molar (N/P) ratios below a defined threshold.

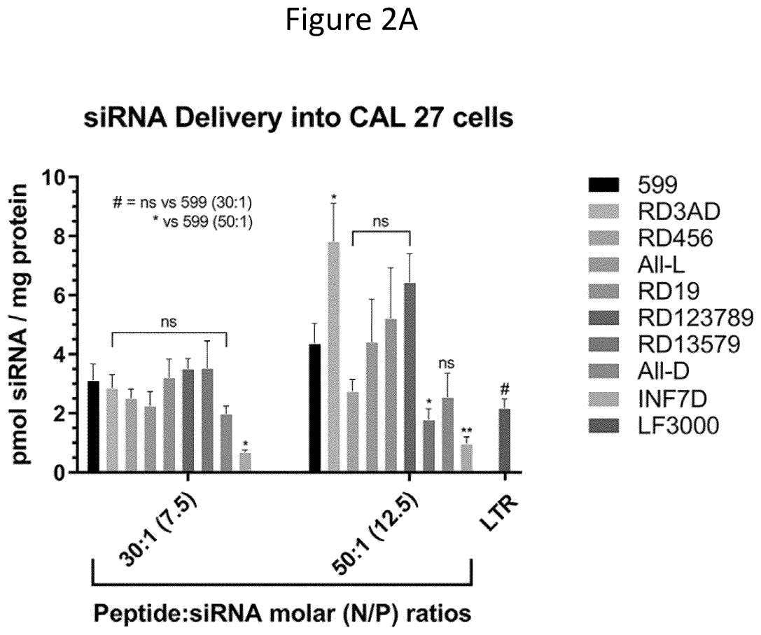

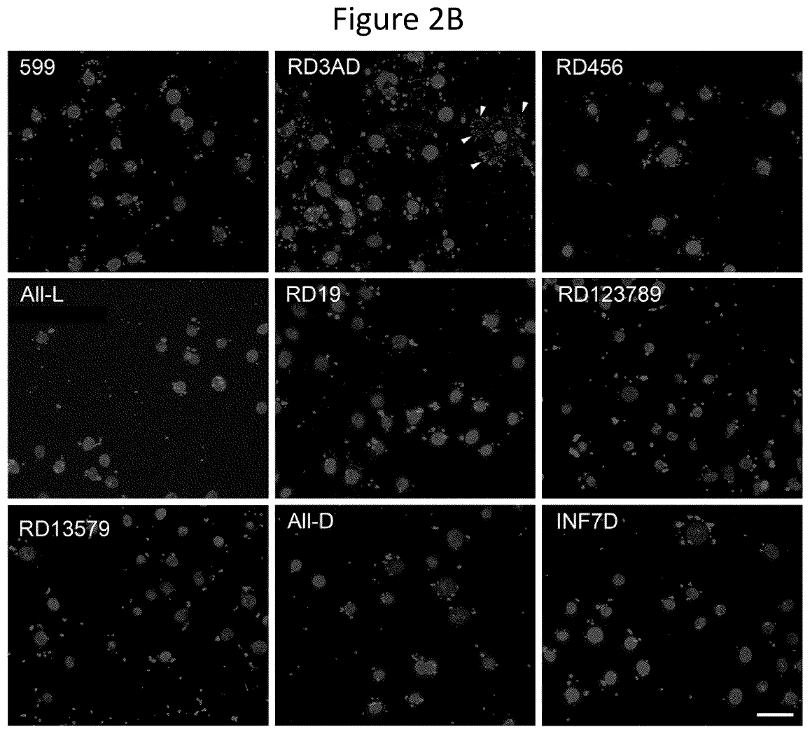

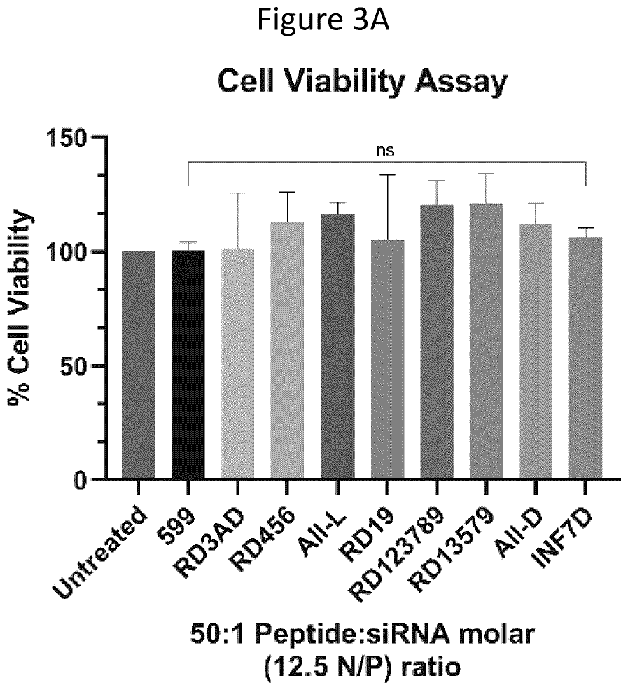

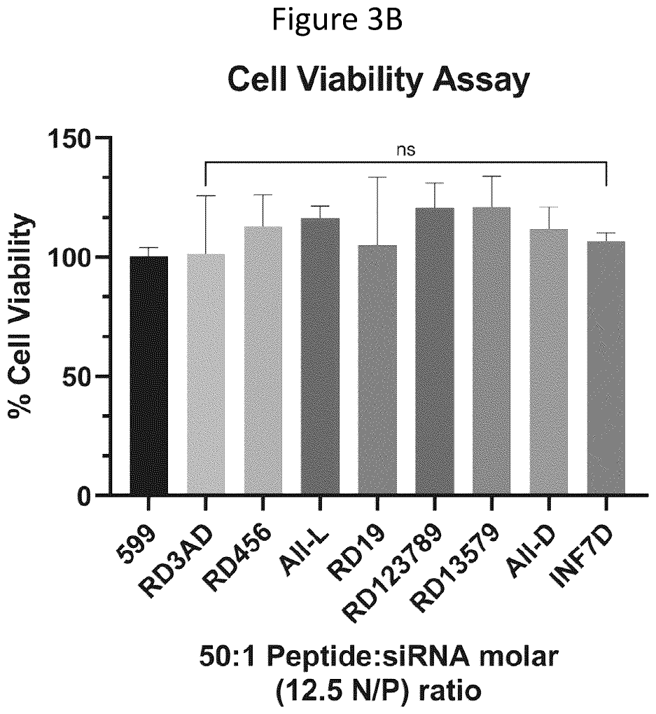

To further examine the effects of the L/D-amino acid stereochemical modifications to the 599 peptide in its ability to deliver siRNAs into cells, next a series of quantitative uptake assays were performed. Previously, it was determined that increasing the Peptide:siRNA molar ratio for the 599 peptide enhanced the delivery of the complexed siRNAs into cells with the 50:1 Peptide:siRNA molar (12.5 N/P) ratio being found to be optimal for the binding and intracellular delivery of siRNAs (Cantini, et al. PLoS One, 2013. 8(9): e73348). Nevertheless, in this study, because ˜100% of siRNAs were found to bind to the 599 peptide and its variants at the 30:1 Peptide:siRNA molar (7.5 N/P) ratio and greater, the decision was made to examine whether any of these variants exhibited improved siRNA cell uptake in comparison to the parent 599 peptide at both the 30:1 and 50:1 Peptide:siRNA molar (7.5 and 12.5 N/P) ratios. In monitoring the quantitative delivery of a DY-547 fluorophore-labeled siCIP2A (DY547-siCIP2A; FIG. 2A), analysis of our data revealed that at the Peptide:siRNA molar ratio of 30:1 (N/P ratio of 7.5), no 599 peptide variant differed significantly from 599 in its ability to deliver siRNAs into CAL 27 cancer cells 2 hours post-treatment with the exception of INF7D, which had a dramatic and significant reduction in siRNA delivery into cells. However, at Peptide:siRNA molar ratios of 50:1 (N/P ratios of 12.5), differences in siRNA uptake became more apparent between the peptide variants with RD19, RD123789, and RD3AD showing on average greater siRNA delivery into cells, but with only RD3AD exhibiting a significantly greater internalization, nearly two-fold higher than 599. Conversely, RD13579, in addition to INF7D, resulted in significantly decreased levels of siRNA cell internalization. As a control, a commercially available lipid-based transfection reagent (LTR), Lipofectamine® 3000 (LF3000), was also used, which was found to deliver siRNAs comparably to 599 and its variants at a Peptide:siRNA molar ratio of 30:1 (N/P ratio of 7.5), but significantly less compared to the 50:1 Peptide:siRNA molar (12.5 N/P) ratio. Visual inspection of the delivery of DY547-siCIP2As into the CAL 27 cancer cells mediated by 599 and its peptide variants (at the 50:1 Peptide:siRNA molar (12.5 N/P) ratio) using confocal fluorescence microscopy (FIG. 2B), demonstrated largely a random punctate uptake pattern, similar to what was previously reported for 599 (Cantini, et al. PLoS One, 2013. 8(9): e73348), with the exception of RD3AD, which exhibited both random and highly ordered linear punctate uptake patterns (for visualization of the latter pattern see arrowheads in the RD3AD panel). Moreover, closer inspection of the RD3AD-treated cells revealed numerous large and small foci in comparison to cells treated with 599 or the other peptide variants, whose foci tended to be larger and fewer in number. Together, these differences in staining patterns for RD3AD were potentially indicative of an alternate mode of cell entry mediated by RD3AD that was more efficient in siRNA delivery, especially in light of the fact that higher levels of siRNA cell internalization were observed for this peptide in the quantitative uptake assays. Nevertheless, these results indicated that varying the peptide stereochemistry patterns within 599 could influence the degree of siRNA delivery into cells, but that a specific D-amino acid substitution within the 599 peptide carrier, as observed through RD3AD, was needed to enhance the cellular internalization of the complexed siRNA cargo. Of note, measurements of the physicochemical properties of the 599 peptide variants in complex with siRNAs at the 50:1 Peptide:siRNA molar (12.5 N/P) ratio, including particle size, zeta potential, and polydispersity index (see Table 2 below), did not reveal any distinguishable characteristics between the complexes that could account for the observed differences in siRNA cellular uptake patterns or that would imply that these differences were a consequence of variances in particle aggregation properties. Nonetheless, based on the above findings, combined with the observation that none of the 599 peptide variants induced long term cytotoxic effects at the 50:1 Peptide:siRNA molar (12.5 N/P) ratio compared to untreated cells (FIG. 3A), nor were they significantly different from 599 (FIG. 3B), this particular Peptide:siRNA molar ratio was used for subsequent experimentations.

TABLE 2
Physicochemical properties of 599 peptide and its
L/D-amino acid stereochemically-modified variants.

| Peptide name | Particle Size (nm)^{a} | Zeta Potential (mV)^{b} | PDI^{c} |
|---|---|---|---|
| 599 | 172.2 ± 81.6 | 30.7 ± 10.6 | 0.349 |
| RD3AD | 213.3 ± 119.3 | 35.3 ± 8.9 | 0.336 |
| RD456 | 200.5 ± 98.5 | 39.2 ± 11.2 | 0.344 |
| All-L | 190.2 ± 82.4 | 39.4 ± 7.3 | 0.327 |
| RD19 | 211.4 ± 98.0 | 31.4 ± 6.7 | 0.381 |
| RD123789 | 224.1 ± 118.4 | 34.1 ± 6.5 | 0.342 |
| RD13579 | 201.2 ± 92.8 | 35.3 ± 7.2 | 0.325 |
| All-D | 185.9 ± 72.0 | 28.3 ± 7.7 | 0.366 |
| INF7D | 190.4 ± 94.6 | 28.2 ± 10.2 | 0.296 |

^{a}Mean hydrodynamic size based on dynamic light scattering measurements. Errors indicate SD from three independent experiments.
^{b}Mean zeta potential measurements. Errors indicate SD from three independent experiments.
^{c}PDI, Polydispersity index

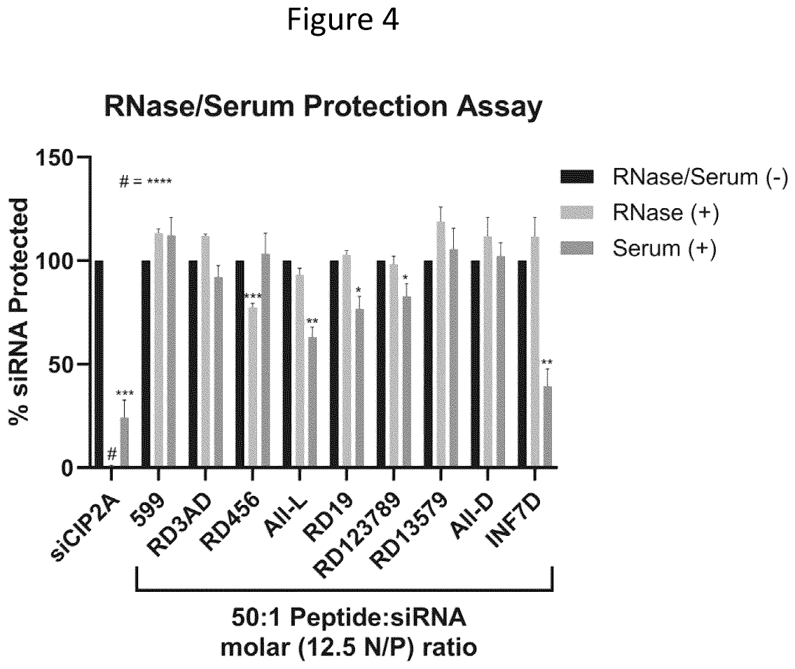

siRNAs delivered in vivo encounter RNases in the physiological environment (e.g. serum) that can degrade them before they reach their therapeutic target (Gavrilov, et al. Yale J Biol Med, 2012. 85(2): 187-200; Alexander-Bryant, et al. J Control Release, 2015. 218: 72-81). Consequently, siRNA-delivery systems must be able to protect the small RNAs from degradation in order for them to maintain their gene silencing function. Previously, 599 had been shown to protect siRNAs from degradation in the presence of RNase A and human serum (Alexander-Bryant, et al. J Control Release, 2015. 218: 72-81). Therefore, to further assess whether L/D-amino acid stereochemical modifications to a peptide carrier could alter its siRNA protective ability, the resistance of 599 and its variants against RNases was assayed by complexing the peptides to siCIP2A and incubating either in the presence of RNase A or human serum for 1 hour (FIG. 4). Data from the assays found that in the presence of RNase A, only RD456 had a significant inability to protect siRNAs, resulting in ˜25% of the complexed siRNAs being degraded. Alternatively, in the presence of human serum, several peptides, including, All-L, RD19, RD123789, and INF7D were significantly less able to protect their complexed siRNAs, with degradation ranging between ˜20-60%. All other 599 peptide variants, including RD3AD, RD13579, and All-D, did not differ from 599 in their ability to protect siRNAs in the presence of either RNase A or serum. Thus, together, these results implied that the stereochemistry of peptide carrier designs does play a critical role in the preservation of complexed siRNAs, with the L/D-arginines within the polyarginine tract of the 599 design, in particular, appearing to be the determinant factors in the sensitivity of the complexed siRNAs to RNase/serum-mediated degradation.

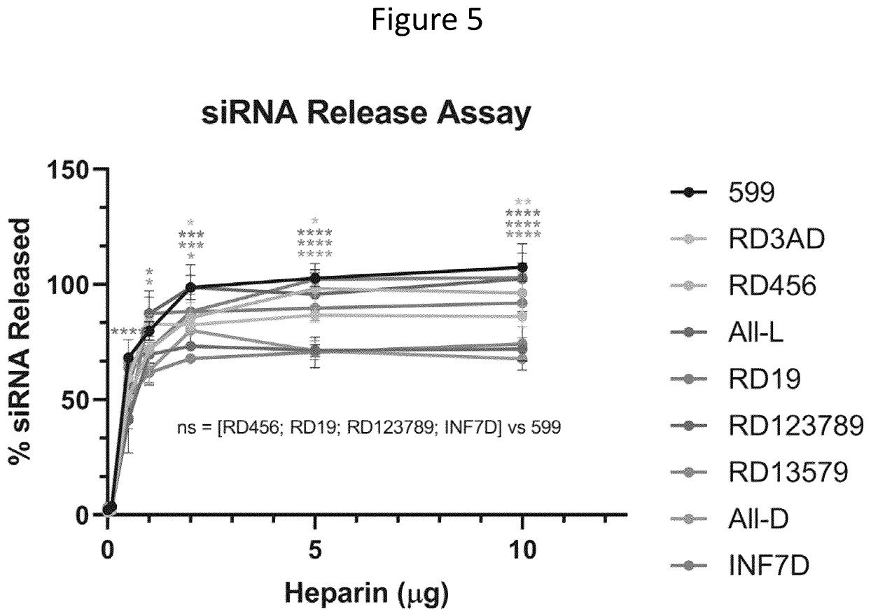

Gene-silencing efficiency is dependent on the release of siRNAs from the delivery complex upon entry into the cell cytoplasm to allow for incorporation of the siRNAs into the RNA-induced silencing complex (RISC) (Jun, et al. PLoS One, 2015. 10(2): e0118310; Ren, et al. ACS Nano, 2012. 6(10): 8620-8631). Thus, in an effort to explore whether specific L/D-amino acid stereochemical modifications could affect siRNA release dynamics, the dissociation of siRNAs from 599 and its peptide variants was assessed by performing quantitative heparin competition assays (FIG. 5). Results from these experiments demonstrated that the siRNAs began to dissociate from all the peptide complexes to varying degrees upon addition of -6 pmol (0.1 μg) of heparin, with 599 capable of releasing approximately 100% of the complexed siRNAs upon increasing competitive amounts of the negatively-charged reagent. No statistical differences in siRNA release were observed for RD456, RD19, RD123789, and INF7D, in comparison to 599. Conversely, RD3AD and All-L/All-D/RD13579 showed significant differences in terms of siRNA dissociation compared to 599, where ˜90% and ˜70% of their complexed siRNAs were maximally released, respectively, at 4-fold excess levels and higher (≥2 μg; ≥120 pmol) of heparin. Intriguingly, two out of the three peptide variants, which exhibited the greatest impairment in siRNA release capability, were the carriers that comprised single amino acid stereochemistries (All-L or All-D). Moreover, although still very effective at releasing siRNAs, substitution of the third D-arginine for D-alanine within the polyarginine tract of RD3AD did not appear to be universal in conferring enhanced siRNA release from a peptide complex (at least compared to the parent peptide), as was reported by Jun et al. in their studies of a multifunctional peptide complex for delivery of siRNAs (Jun, et al. PLoS One, 2015. 10(2): e0118310). Nonetheless, the fact that 6 out of 7 peptides that comprised both L and D-amino acid sequence patterns showed ≥90% siRNA release, suggested that the incorporation of alternating amino acid stereochemistries within peptide carrier designs appeared to be an important factor in ensuring efficient siRNA release from the complex.

L/D-Amino Acid Stereochemical Modifications to the 599 Peptide siRNA-Carrier Affect the Gene Silencing Properties of the Complex

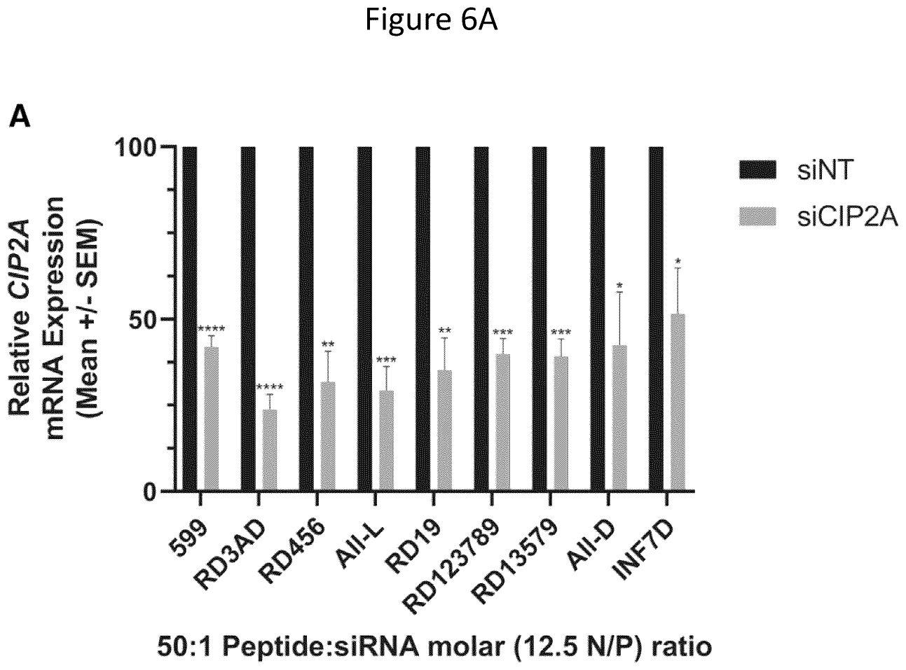

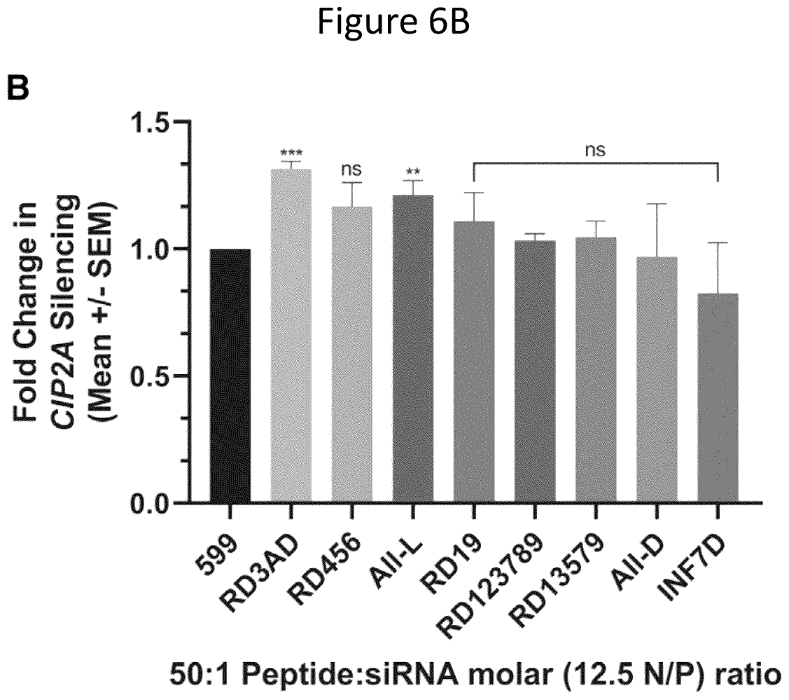

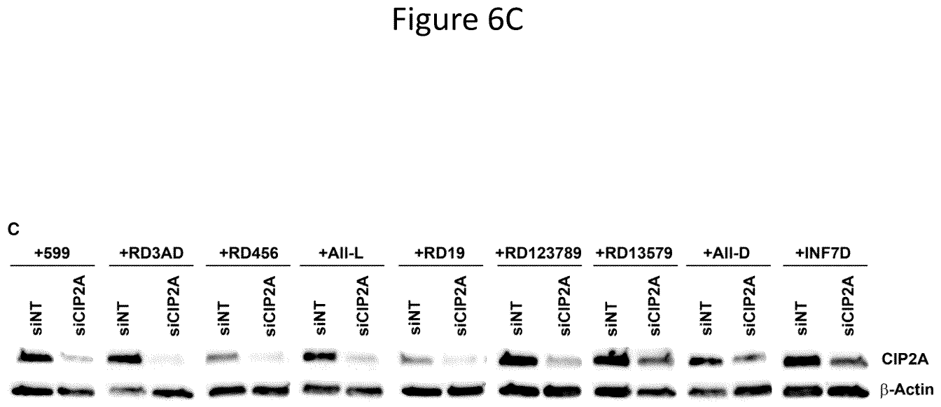

599 has previously been shown to be an effective carrier in the delivery of siRNAs that could induce both in vitro and in vivo gene silencing (Cantini, et al. PLoS One, 2013. 8(9): e73348; Alexander-Bryant, et al. J Control Release, 2015. 218: 72-81). Nonetheless, with the above findings that specific L/D-amino acid stereochemical changes could affect the binding, cell internalization, protection, and release of siRNAs mediated by 599, it made evident the need to further assess the gene silencing functionality of the 599 peptide variants in complex with siRNAs. By doing so, this would help ascertain whether these properties were related to enhancements in gene silencing and whether there was potential for the advancement in the peptide carrier design. Consequently, to assess gene silencing efficiencies mediated by 599 and its peptide variants, CAL 27 cancer cells were treated with 599 or its peptide variants complexed with either a control non-targeting siRNA (siNT) or siCIP2A, after which the CIP2A mRNA levels were assessed 48 hours post-treatment by real-time PCR. Quantitation of the results demonstrated significant knockdowns in CIP2A mRNA levels for all the peptides tested, ranging between ˜50-80%, with RD3AD having the highest observed reduction in CIP2A mRNA levels at approximately 80% (FIG. 6A). However, in comparison to 599, only two peptide variants, RD3AD and All-L, were found to exhibit significant enhancements in gene silencing, with RD3AD showing the greatest improvement at ˜30% (FIG. 6B). To further corroborate the real-time PCR results, Western blot analyses were performed, which confirmed the ability of the 599 peptide variants to deliver functional siRNAs by demonstrating the suppression of CIP2A protein levels 48 hours post-treatment (FIG. 6C). Intriguingly, the data also appeared to support RD3AD as having the highest observed reduction in CIP2A protein levels amongst the peptide variants in comparison to 599. Thus, these findings demonstrated that the gene silencing functionality of the 599-siRNA complex could be influenced by varying the stereochemical patterns within the peptide carrier design, but that a specific D-amino acid substitution, as observed through RD3AD, was particularly significant because it revealed its potential in advancing the 599 peptide carrier design for siRNA-based therapeutics.

RD3AD-siRNA Complexes Colocalize with Filopodia on Cell Sur Faces

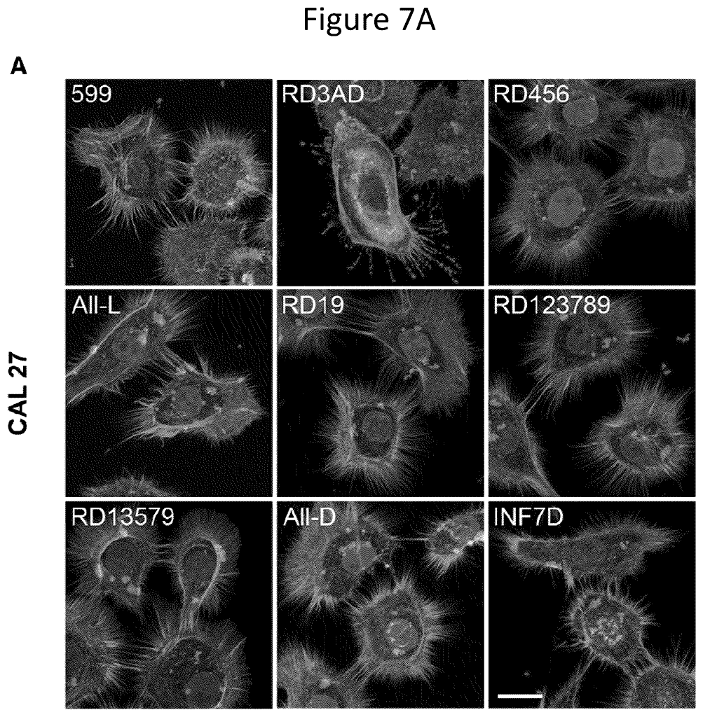

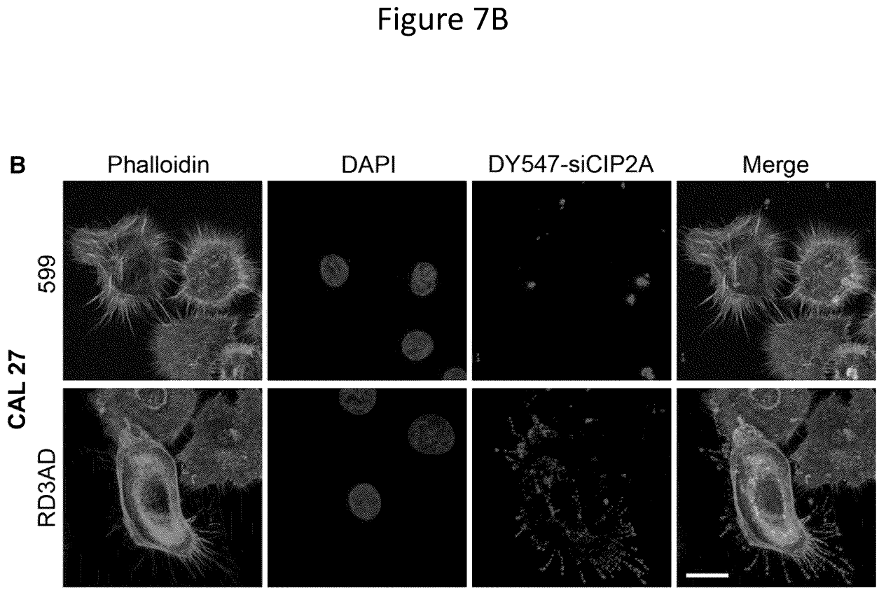

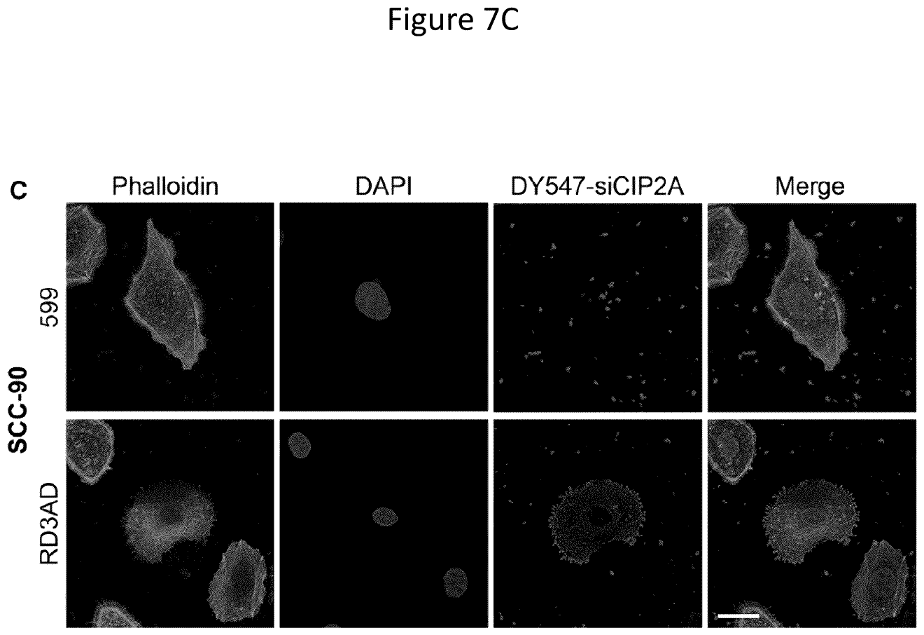

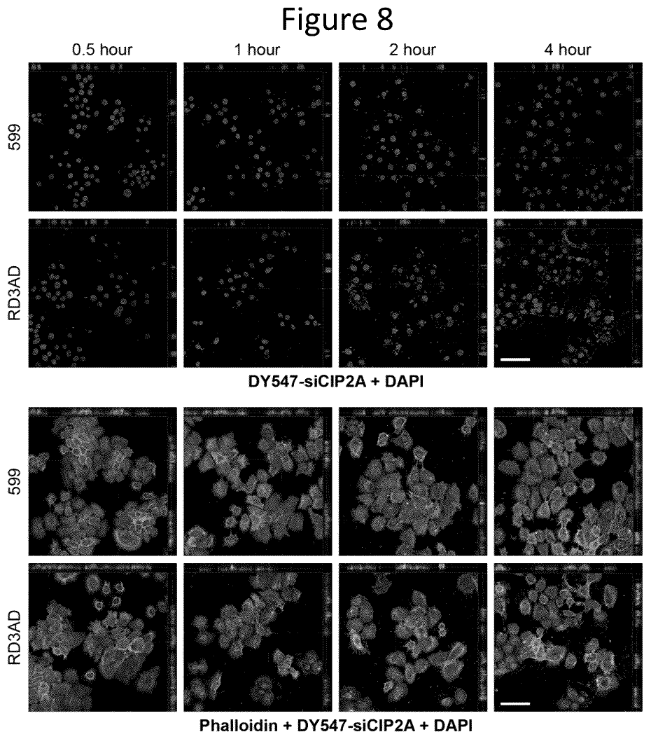

Having found that RD3AD in complex with siRNAs could enhance gene silencing and that this peptide mediated increased cellular internalization of the complexed siRNA cargo with an apparent highly ordered linear accumulation of siRNA-containing foci at what appeared to be the cell periphery, implied that its mode of cell entry was potentially more efficient compared to 599 and the other peptide variants. Thus, in an effort to better resolve these structures and elucidate a mechanism responsible for the improved uptake of siRNAs and consequently, the enhanced gene silencing mediated by RD3AD, the delivery of fluorophore-labeled siRNAs into cells mediated by 599 and its peptide variants were assessed at a high magnification using confocal fluorescence microscopy (FIG. 7A). Results from these experiments verified that treatment of CAL 27 cancer cells with 599 or its peptide variants in complex with DY547-siCIP2As, predominantly exhibited irregular-shaped foci of varying sizes (most of which were on the larger side) that localized randomly to both the periphery of cell membranes, as well as the cytoplasm, 2 hours post-treatment. Notably, the exception was RD3AD, which showed a highly ordered accumulation of uniformly-shaped spherical foci that lined up along cell surface protrusions, identified as filopodia. Interestingly, filopodia are highly dynamic, elongated, and thin cellular processes that have been reported to facilitate the highly efficient cell entry of viruses, bacteria, activated receptors, lipo/polyplexes, and exosomes (Heusermann, et al. J Cell Biol, 2016. 213(2): 173-184; Lehmann, et al. J Cell Biol, 2005. 170(2): 317-25; Lidke, et al. J Cell Biol, 2005. 170(4): 619-26). Thus, the association of the RD3AD-siRNA complex with filopodia could explain why a greater accumulation of small siRNA-containing foci were also detected in the cytoplasm of treated cells versus the 599-siRNA complex, which conversely did not appear to localize along filopodia and only showed a limited uptake of its siRNA cargo in mostly large punctate structures (FIG. 7B). Further examination into whether the staining pattern for RD3AD-mediated siRNA uptake was reproducible in another cancer cell line likewise demonstrated that treatment of SCC-90 cells with either RD3AD or 599 in complex with DY547-siCIP2As produced the same staining pattern differences observed for both peptide carriers in CAL 27 treatments (FIG. 7C). More specifically, the RD3AD-siRNA complexes were found to exhibit the same dramatic “beads on a string” pattern along flilopodia, in addition to an accumulation of numerous small cytoplasmic foci, whereas 599-siRNA complexes were predominantly larger, irregularly-shaped structures, that were fewer in number and localized to both the cytoplasm and periphery of the cell membrane with no apparent associations with filopodia. The fact that the staining pattern we observed for RD3AD-mediated siRNA uptake in both cancer cell lines was very reminiscent of the patterns previously reported for cell entry of viruses, bacteria, activated receptors, lipo/polyplexes, and exosomes (Heusermann, et al. J Cell Biol, 2016. 213(2): 173-184; Lehmann, et al. J Cell Biol, 2005. 170(2): 317-25; Lidke, et al. J Cell Biol, 2005. 170(4): 619-26), implied that this peptide carrier could potentially exploit similar cell machineries for efficient delivery of its cargo by using filopodia to gain entry into cells, thereby contributing to the enhancement of gene silencing. In effect, time course analyses of cells treated with RD3AD in complex with DY547-siCIP2A clearly demonstrated an accumulation of siRNAs along filopodia, the cell periphery, and within the cell over a period of 4 hours in comparison to cells treated 599-siRNA complexes with the most noticeable differences observed at the 4 hour time period (FIG. 8). Moreover, cross sectional analyses of the treated cells at the 4 hour time period corroborated the increased uptake of siRNAs within the cells, as evidenced by the greater degree of siRNA colocalization with cytoplasmic F-actin, which helped demarcate the cell body in addition to filopodia. Conversely, 599 treatments appeared to show the siRNAs predominantly localized on the surfaces of cell bodies with limited intracellular uptake. Thus, together, these data strengthened the notion that RD3AD appeared to mediate a more efficient mode of cell entry possibly through its associations with filopodia.

Peptide Carrier Stereochemistry Modifications and D-Amino Acid Substitutions Impact siRNA Delivery and Gene Silencing

The results presented here highlight the importance of peptide stereochemistry, in particular, the modification of specific L/D-amino acid sequence patterns in peptide carrier designs, with regards to siRNA complexation, intracellular delivery, RNase/serum sensitivity, complex release, and gene silencing efficiency. Moreover, our findings notably demonstrate that a peptide carrier through a D-amino acid substitution can enhance siRNA delivery and consequently gene silencing via potential “surfing” along filopodia. As stated previously, conventional thought was that stereochemistry in peptide carrier designs was primarily used to confer proteolytic resistance function to the peptide-siRNA complex, making the peptide and its therapeutic cargo more stable for in vivo therapeutic applications. However, Zeller et al. alternatively found that the proteolytic digestion of the peptide carrier in endosomes was actually critical to prolonging the dynamics of gene silencing due to the enhanced release of siRNAs from late endosomes to the cytosol (Zeller, et al. Chem Biol, 2015. 22: 50-62). Thus, these data made evident the need for a balance in the design of peptide carriers when modulating their L/D-amino acid stereochemistries. Additionally, in studies by Verdurmen et al. and Favretto et al., it had been demonstrated that modulation of peptide stereochemistry could also affect the cell uptake efficiency of both free arginine-rich-containing CPPs and that of antisense oligonucleotide-bound CPP polyplexes (Verdurmen, W P, et al., Chem Biol, 2011. 18(8): 1000-1010; Favretto, M E, and Brock, R, Small, 2015, 11: 1414-1417), further implying that peptide stereochemistries could potentially confer additional functions, beyond the prototypical protease resistant function. Accordingly, to further expand our knowledge in the application of stereochemistry in peptide carrier designs, this study sought to determine how specific L/D-amino acid stereochemical modifications within the 599 peptide carrier, which we previously designed, may impact peptide carrier properties/functions and advance the 599 peptide carrier design for the enhancement of gene silencing in cancer cells.

Because the 599 peptide was designed to electrostatically complex with siRNAs, (Cantini, et al. PLoS One, 2013. 8(9): e73348) whether alteration of L/D-amino acid sequence patterns, as well as a specific D-arginine substitution for D-alanine would alter the binding affinity of the 599 peptide carrier, was initially examined. Results from this Example found that these modifications to the 599 peptide could indeed alter the ability to bind free siRNAs in a dose dependent manner, with all peptide variants binding ˜100% of siRNAs at ≥30:1 Peptide:siRNA molar ratios. Although there were no differences in siRNA binding at this particular Peptide:siRNA molar ratio and greater, most peptides (with the exception of All-L and RD123789) did bind significantly better than 599 between the 1:1 and 20:1 Peptide:siRNA molar ratios, with RD456 showing the most significant improvement overall. Moreover, 6 out of the 8 peptide variants exhibited the highest differences in binding ability at the 10:1 molar ratio, with RD456 and INF7D showing a ˜20% binding improvement relative to 599. This finding is particularly interesting because 599, which has been previously determined to function optimally at the 50:1 Peptide:siRNA molar ratio, is typically formulated by mixing the relative molar amounts of both the peptide and siRNA in one reaction (Cantini, et al. PLoS One, 2013. 8(9): e73348). Therefore, one could potentially envision improving the siRNA binding capacity of the complex, by complexing RD456, for example, by sequentially adding the peptide five times to the siRNA (with the starting siRNA amount increased by ˜20%) at 10:1 Peptide:siRNA molar ratio increments, to allow it to function at the optimal Peptide:siRNA molar ratio.

Subsequent investigation into the intracellular delivery of siRNAs mediated by the 599 peptide variants, revealed that even though all the peptides bound ˜100% of the siRNAs at the 30:1 Peptide:siRNA molar ratio, increasing the Peptide:siRNA molar ratio to 50:1 further increased siRNA uptake into cells 2 hours post-treatment in comparison to naked siRNAs for 5 out of 7 peptides, in addition to 599, which was consistent with previously published data pertaining to 599(Cantini, et al. PLoS One, 2013. 8(9): e73348), along with other reports that similarly found that increasing CPP concentrations typically resulted in increased siRNA uptake ability (Lundberg, et al. Faseb j, 2007. 21: 2664-2671; Kumar, et al. Nature, 2007. 448: 39-43). Regarding the stereochemical changes to the 599 peptide design, alteration of L/D-amino acid sequence patterns was found to only adversely affect the intracellular delivery of siRNAs for two peptides, INF7D and RD13579, in comparison to 599, but otherwise these changes were largely tolerable, with no significant differences observed for the other 5 peptides in comparison to 599. These data did, however, make evident that changing the chirality of the fusogenic INF7 N-terminal region to the non-natural D-amino acids was potentially detrimental to the uptake of siRNAs, as observed through All-D and INF7D, which both showed on average lower siRNA uptake abilities in comparison to 599, suggesting that this type of modification was to be avoided, possibly due to its functional importance in membrane destabilization (Plank, et al. J Biol Chem, 1994. 269: 12918-12924). Notably, the only peptide that showed a dramatic and significant increase in intracellular siRNA delivery, which was nearly two-fold higher than 599, was RD3AD, which was modified with a specific D-arginine substitution for D-alanine, and whose design was based on evidence that a similar modification within a receptor-targeting CPP could lead to enhanced gene silencing mediated by a multifunctional peptide complex (Jun, et al. PLoS One, 2015. 10(2): e0118310). Interestingly, confocal fluorescent microscopy analyses of the cellular uptake patterns for both the stereochemically-modified peptides and RD3AD in complex with fluorophore-labeled siRNAs, revealed that all the peptide variants, with the exception of RD3AD, exhibited predominantly random punctate patterns, whose foci tended to be larger and fewer in number, similar to 599, the difference being that RD3AD also showed highly ordered linear punctate uptake patterns, as well as both numerous large and small foci. Thus, these differences in cellular uptake patterns for RD3AD could possibly account for the higher detectable levels of siRNAs found within cells treated with RD3AD-siRNA complexes and could be indicative of an alternate and more efficient mode of cell entry compared to 599 and the other peptide variants.

In further analyzing the effects of the stereochemical modifications and the D-amino acid substitution on 599 peptide properties, in terms of cytotoxicity, none of the peptide variants differed significantly in their ability to affect the long-term viability of treated cells compared to 599, nor to untreated cells (with the latter comparison also true for 599, which corroborated the previous findings of Cantini, et al. that 599 treatments in complex with siRNAs were non-cytotoxic). Conversely, regarding siRNA stability, the data did, however, suggest that these modifications did play a critical role in the preservation of complexed siRNAs, with the L/D-arginines within the polyarginine tract of the 599 design, in particular, appearing to be the determinant factors in the sensitivity of the complexed siRNAs to RNase/serum-mediated degradation. Intriguingly, the central arginine residues (#4, 5, and 6) of the nona-arginine tract appeared to be key to determining the stability of the complex, as RD456 was found to be the only peptide variant whose complexed siRNA was susceptible to direct RNase treatment, resulting in ˜25% siRNA degradation. Furthermore, in the presence of serum (which contains both proteases and nucleases [Whitehead, et al. Nat Rev Drug Discov, 2009. 8(2): 129-138; Pujals, et al. Biochem Soc Trans, 2007. 35(Pt 4): 794-796; Guidotti, et al. Trends Pharmacol Sci, 2017 38: 406-424]), the fact that peptides All-L, RD19, RD123789, and INF7D, which all had L-arginines at positions #4, 5, and 6 of the nona-arginine tract, were significantly less able to protect their complexed siRNAs compared to 599, with degradation ranging between ˜20-60%, whereas RD456 could, indicated that the incorporation of L-amino acid chirality at these three positions was to be avoided, as it would make the complex more susceptible to proteolytic digestion, resulting in its destabilization, with a consequent exposure of the siRNAs to serum nucleases. Thus, it appeared that because the RD456 design renders the peptide susceptible to RNase-mediated degradation of its siRNA cargo and maintaining L-arginines within the central region of the polyarginine tract (All-L, RD19, RD123789, and INF7D) renders the peptide susceptible to protease degradation, these particular modifications were to be avoided in any advancement of 599 peptide designs. The data pertaining to siRNA release also indicated that the incorporation of alternating amino acid stereochemistries within the 599 peptide carrier design appeared to be an important factor in ensuring efficient siRNA release from the complex, accounting for ≥90% siRNA release, as 2 out of 3 peptides that had the poorest release capabilities comprised single amino acid stereochemistries (All-L or All-D). Interestingly, this parity in poor siRNA release capabilities between All-L and All-D corroborated the findings from an earlier study by Favretto et al. (Favretto, M E, and Brock, R, Small, 2015, 11: 1414-1417), which likewise found that polyplexes comprising either all D- or all L-CPPs were less likely to fall apart and were equally stable in the presence of heparin when formulated at high peptide concentrations. Taken together, these data also potentially implied that the poor siRNA release mediated by All-L and All-D were the consequence of greater complex stability conferred by the single amino acid stereochemistry peptide designs. However, it should be noted that alternating the chirality for every other amino acid within the polyarginine tract, which is responsible for electrostatically complexing the siRNAs (Cantini, et al. PLoS One, 2013. 8(9): e73348), was also found to be equally poor at releasing siRNAs from the complex. Surprisingly, although still very effective at releasing siRNAs, substitution of the third D-arginine for D-alanine within the polyarginine tract of RD3AD did not universally confer enhancement of siRNA release from the peptide complex (at least compared to the parent peptide), as was reported by Jun et al. in their studies of a multifunctional peptide complex for delivery of siRNAs (Jun, et al. PLoS One, 2015. 10(2): e0118310). Thus, the introduction of an uncharged amino acid into the polyarginine tract, to decrease the electrostatic interactions between the siRNA and peptide carrier, does not necessarily result in greater release of the siRNA. Interestingly, the Jun et al. study, however, never made clear the chirality of the substituted alanine residue in their peptide carrier design, thus, opening the possibility that this discrepancy in findings could be related to amino acid stereochemistry differences.

Despite the differences in the functional properties described above, the modification of L/D-amino acid sequence patterns in the 599 peptide carrier design still resulted in effective gene silencing of the targeted CIP2A oncogene, ranging between ˜50-70% knockdown. However, it was RD3AD, with its specific D-amino acid substitution, that showed the greatest silencing at ˜80%. Moreover, although most peptide variants showed improvements, only RD3AD and All-L were found to be significantly more efficient, ˜30% and ˜20%, respectively at gene silencing than 599. The observation that All-L was more efficient at gene silencing compared to 599 was not unexpected considering the Zeller et al. study, which reported that L-arginine-rich CPPs, but not D-arginine-rich CPPs, could prolong the dynamics of gene silencing due to the requirement for partial degradation of arginine-rich CPPs by endosomal proteases in order to enable more effective endosome-to-cytosol translocation of the complexed siRNAs (Zeller, et al. Chem Biol, 2015. 22(1): 50-62). The fact that the All-L-siRNA complex was found to be unstable in serum compared to 599-siRNA complexes, suggested that its design was more readily susceptible to proteolytic degradation and therefore better at translocating siRNAs from the endosome to the cytosol. The same could not be said for INF7D, however, which also comprised an all L-amino acid polyarginine tract and whose complex had the greatest instability in serum, but at the same time also had the poorest siRNA cell uptake that most likely counteracted and contributed to it having the weakest gene silencing effect among the peptide variants.

Finally, the findings that RD3AD exhibited the most enhanced siRNA delivery and further corroborated by 2D/3D confocal fluorescence imaging, and consequently greater gene silencing were significant because they implied that its mode of cell entry was potentially more efficient compared to 599 and the other peptide variants. In fact, the discovery that RD3AD-siRNA complexes could localize along filopodia on cell surfaces supported this notion, especially in light of the fact that viruses, activated receptors, lipo/polyplexes, and exosomes have been reported to bind to filopodia and induce a rapid, but directed, retrograde “surfing” movement towards the cell body for entry into cells (Heusermann, et al. J Cell Biol, 2016. 213(2): 173-184; Lehmann, et al. J Cell Biol, 2005. 170(2): 317-25; Lidke, et al. J Cell Biol, 2005. 170(4): 619-26; ur Rehman, et al. ACS Nano, 2012. 6: 7521-7532). Particularly noteworthy, is that at the filopodial base are endocytic hot spots, which have been described, as active areas of actin remodeling that potentially allow for easier cell entry, in comparison to other sites along the cell membrane that are more difficult to penetrate due to the dense cortical actin cytoskeleton (Lehmann, et al. J Cell Biol, 2005. 170(2): 317-25). Interestingly, in the case of exosomes, they have been found to sort into endosomal trafficking circuits at the base of filopodia that are targeted to the endoplasmic reticulum (ER), as a possible site of cargo release (Heusermann, et al. J Cell Biol, 2016. 213(2): 173-184). Coincidentally, the ER has also been identified as the central nucleation site of siRNA-mediated silencing (Stalder, et al. EMBO J, 2013. 32: 1115-1127). Thus, this directed transport along filopodia to endocytic hot spots, followed by endocytosis and trafficking to the ER could potentially allow for the efficient entry of siRNA cargo to cellular RNAi machinery that could be exploited by peptide carriers.

In conclusion, our data collectively demonstrate the utility of peptide stereochemistry, as well as the importance of a key D-amino acid modification in advancing the 599 carrier design for the enhancement of gene silencing in cancer cells. Moreover, this study makes evident the need for a balance in the design of peptide carriers when modulating their stereochemistry, but also demonstrates the potential in fine-tuning peptide carrier properties/functions by altering their L/D-amino acid sequence patterns. It will be interesting in future studies to further delineate the mechanisms of cell uptake and expand upon how peptide stereochemistry and/or the specific D-alanine substitution in the 599 peptide carrier design might exploit host cell machineries like filopodia to gain more efficient siRNA drug entry into cells. Additionally, future studies will also need to confirm the direct cell uptake action of filopodia in mediating the intracellular delivery of RD3AD-siRNA complexes, which could be tested through the use of several chemical inhibitors, such as cytochalasin D and SMIFH2, which are known inhibitors of filopodia-mediated retrograde trafficking and filopodia structures, respectively (Heusermann, et al. J Cell Biol, 2016. 213(2): 173-184; Rizvi S A, et al., Chem Biol, 2009, 16(11):1158-1168; Barry D J, et al., J Cell Biol, 2015, 209(1):163-180; Isogai T, et al., Sci Rep, 2015, 5:9802). Furthermore, because lipo/polyplexes have been reported to use syndecan-dependent transport mechanisms in filopodia to reach the cell surface, one could also envision testing whether heparinase and sodium chlorate, which are known disruptors of syndecans (ur Rehman Z, et al., ACS Nano, 2012, 6(8):7521-7532), can likewise impair filopodia-mediated cell uptake of RD3AD-siRNA complexes. Nonetheless, the future study of these modifications in peptide carrier-siRNA cargo complex formation/disassembly and function from both a mechanobiology and 3D structural perspective are just as enticing, which could lead to further advancement of the 599 peptide carrier design and its prospective translation to the clinic, as a delivery vehicle for siRNA-based human cancer therapies.

### Example 2: Chirality of 599 Peptide Derivatives and Localization to Filopodia

In an effort to delineate the importance of the specific D-amino acid modification within the RD3AD peptide (see Table 1 of Example 1 above; also see SEQ ID NO: 20 in Table within the Compositions section above) in mediating localization of complexed siRNAs to filopodia found on oral cancer cells, a derivative of RD3AD that incorporated an L-alanine substitution in place of the D-alanine, which was termed RD3AL, was designed and synthesized (see SEQ ID NO: 20 in Table within the Compositions section above). The priority was to determine first whether the chirality of the substituted alanine residue within the RD3AD design was key to mediating associations with filopodia and whether this change would affect gene silencing efficiencies relative to RD3AD.

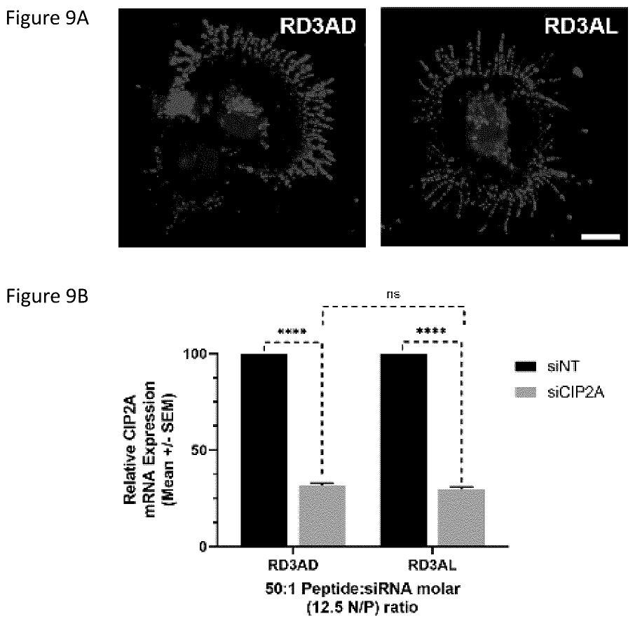

Upon treating CAL 27 oral cancer cells with a DY547-labeled siCIP2A (DY547-siCIP2A) in complex with either RD3AD or RD3AL, it was found that RD3AL was equally capable of producing highly ordered linear accumulations of uniformly-shaped spherical siRNA-containing foci along cell surface projections, as well as accumulations of siRNAs within the cytoplasm of cells, similar to RD3AD (FIG. 9A). The linear accumulation of the siRNA-containing foci along the cell surface projections were subsequently determined to be co-localized with filopodia (data not shown). Ensuing gene silencing experiments (FIG. 9B) further demonstrated that cancer cells treated with RD3AL-siCIP2A complexes could mediate significant CIP2A knockdown in comparison to RD3AL in complex with a control non-targeting siRNA (siNT) and that the silencing was comparable to that observed by RD3AD-siCIP2A complexes. Additionally, the fact that there were no differences observed between the two peptides in their abilities to bind, release, and protect siRNAs (data not shown), implied that the chirality of the substituted alanine residue within the original RD3AD design was not critical in mediating its unique properties. However, this does not rule out whether alanine positioning within the nonaarginine tract of the RD3AD design is still a factor.

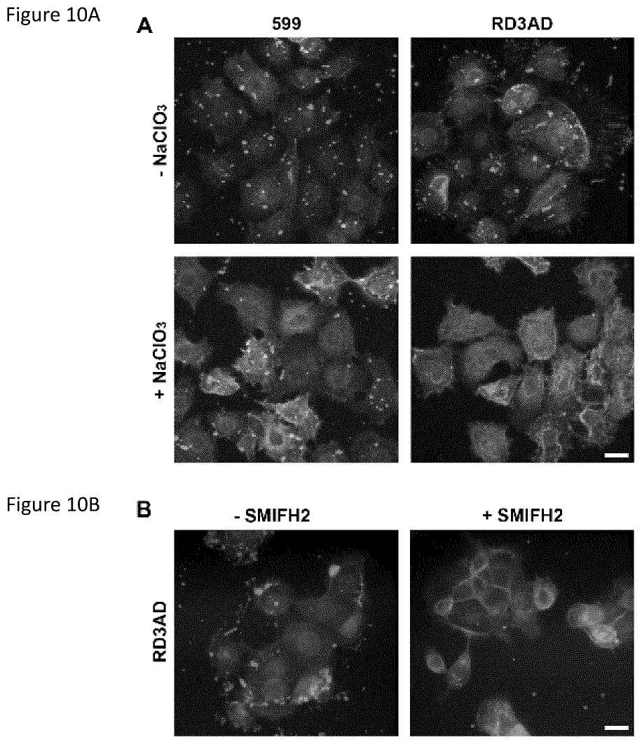

Next, analyses were conducted to determine the direct role filopodia might play in the cellular uptake of RD3AD and its peptide variants in complex with siRNAs. Interestingly, pre-treatment of cancer cells with sodium chlorate, which inhibits sulfation of heparin sulfate proteoglycans (HSPGs) (ur Rehman Z, et al., ACS Nano, 2012, 6(8):7521-7532), including syndecans, which are a family of HSPGs known to localize to filopodia and mediate retrograde transport (ur Rehman Z, et al., ACS Nano, 2012, 6(8):7521-7532 Schelhaas M, et al., PLoS Pathog, 2008, 4(9):e1000148), impaired the ability of RD3AD to interact with filopodia and deliver siRNAs into cells (FIG. 10A). Conversely, the 599 peptide, which does not associate with filopodia (see FIG. 7 of Example 1 above), was unaffected in its ability to deliver complexed siRNAs into cells. Additionally, pre-treatment of cancer cells with the inhibitor SMIFH2, which results in loss of filopodia (Heusermann, et al. J Cell Biol, 2016. 213(2): 173-184; Rizvi S A, et al., Chem Biol, 2009, 16(11):1158-1168; Barry D J, et al., J Cell Biol, 2015, 209(1):163-180; Isogai T, et al., Sci Rep, 2015, 5:9802), abrogated the ability of RD3AD to deliver siRNAs to cells (FIG. 10B). Consequently, the importance of these findings are that they provide the first evidence for the direct role of filopodia in mediating the intracellular delivery of peptide carrier-siRNA complexes. Moreover, these data also demonstrate that specific 599 peptide derivatives, such as RD3AD/L, can be designed to specifically target filopodia on cell surfaces, which could prove instrumental in the development of more effective targeted-delivery platforms for cancer interventions, especially with respect to metastatic cells, which have an abundance of filopodia (Han S, et al., Mol Oncol, 2016, 10(7):966-980; Coopman P J, et al., Clin Cancer Res, 1998, 4(2):507-515; Wang W, et al., Cancer Res, 2002, 62(21):6278-6288).

### Example 3: RD3AD Enhances siRNA Delivery and Gene Silencing in Dual Peptide System

Previous studies have shown that the EGFR-targeting GE11R9 peptide was able to deliver siRNAs specifically to EGFR-overexpressing cells, but siRNAs were incapable of escaping endosomes to mediate effective gene silencing (Alexander-Bryant, et al. Oral Oncol, 2017. 72: 123-131). However, when used in conjunction with the endosome-disruptive 599 peptide, the dual peptide system resulted in both efficient targeting of the siRNA to EGFR-overexpressing cells and silencing of the siRNA-specific oncogene (Alexander-Bryant, et al. Oral Oncol, 2017. 72: 123-131). As a follow up to the findings of Example 1 that RD3AD exhibited the most enhanced siRNA delivery and gene silencing of the 599 derivatives studied, the effects of RD3AD when used in conjunction with GE11R9 as part of a dual peptide delivery system were explored.

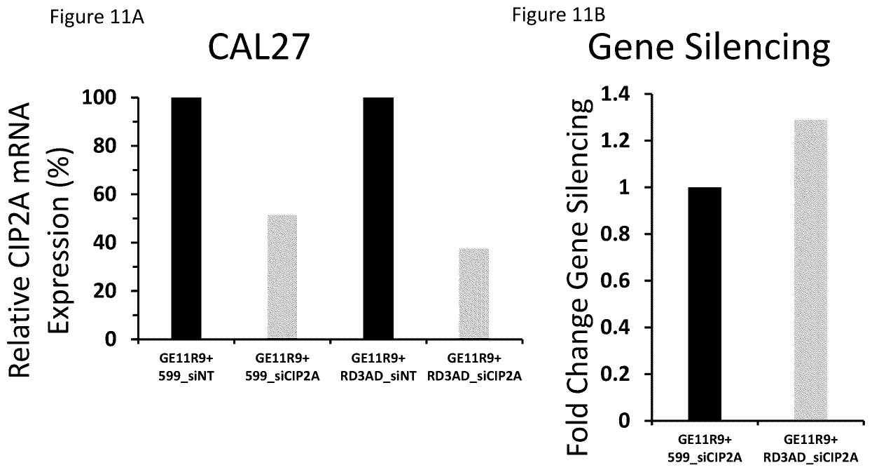

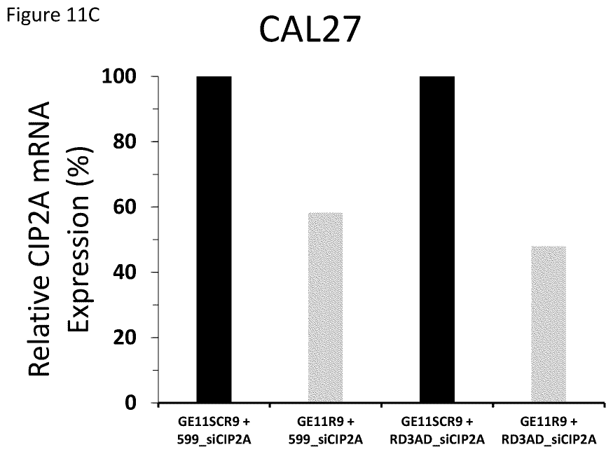

As shown in FIG. 11A, RD3AD in conjunction with GE11R9 demonstrated siCIP2A delivery into EGFR-overexpressing CAL 27 cancer cells and ˜62% knockdown of CIP2A mRNA. By comparison, 599 and GE 11R9 resulted in ˜48% knockdown of CIP2A mRNA. This corresponds to a fold change in gene silencing of ˜30% for RD3AD relative to 599 when both are used in conjunction with GE11R9 (FIG. 11B). Finally, the effects of GE11R9 were shown to be specific, as both RD3AD and 599 were substantially more efficient at knocking down CIP2A in CAL 27 cells when used in conjunction with GE11R9 as compared a scrambled control peptide, GE11SCR9 (FIG. 11C). Notably, the increased efficiency of RD3AD versus 599 peptide was maintained even when normalized to the scrambled peptide.

These results demonstrate that the findings of Example 1, wherein 599 peptide was further optimized as an siRNA delivery system by making D-amino acid substitutions and altering stereochemistry, translate to the dual peptide system. More specifically, the results suggest that RD3AD, which was shown to be a more efficient siRNA delivery vehicle than 599, maintains this increase in efficiency when used in conjunction with a targeting peptide, such as GE11R9. One of skill in the art could thus appreciate how 599 derivatives of the present invention, when used in conjunction with a targeting peptide, such as GE11R9, could be used to efficiently deliver siRNA to specific cell types in vivo as a form of cellular therapy to treat or prevent diseases or disorders.

### Example 4: 599 Interacts Similarly with Multiple siRNA Species

As demonstrated in Examples 1, 2, and 3, 599 and its derivatives are capable of efficient binding to siCIP2A, particularly at higher peptide:siRNA molar ratios (>30:1). This allows for efficient delivery of siCIP2A into cells and subsequent knockdown of the tumor oncogene CIP2A. As a follow up to these Examples, the binding of alternative siRNAs to 599 were investigated to determine if the observed results were likely siRNA-specific or more broadly applicable to a wide range of siRNA species.

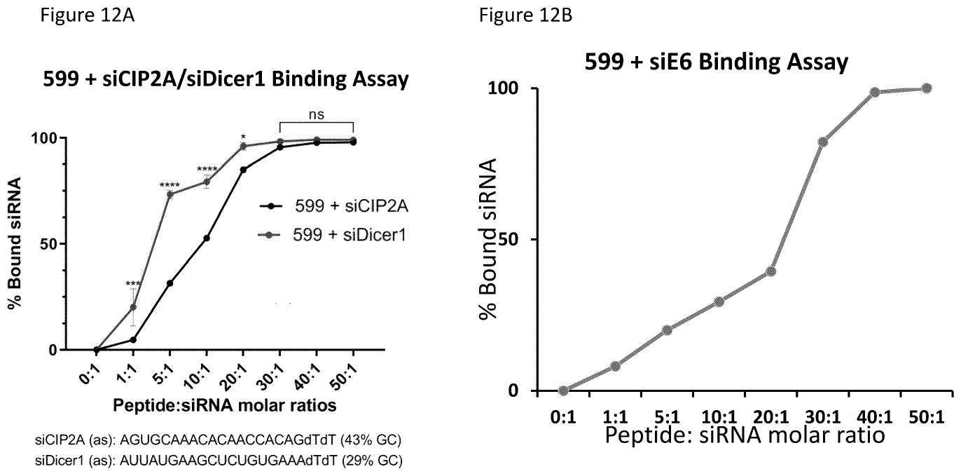

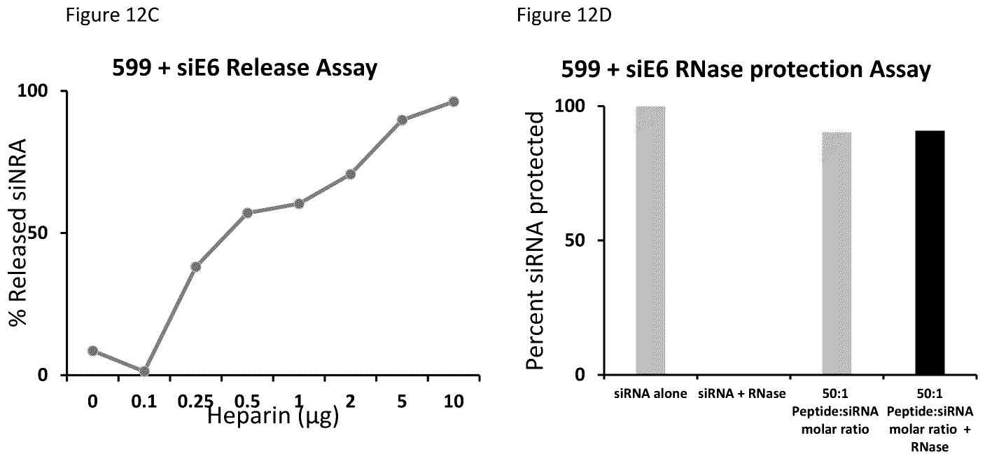

As shown in FIG. 12A, both siCIP2A and the unrelated siRNA targeted to Dicer1, siDicer1, are able to bind similarly to 599 peptide at peptide:siRNA molar ratios greater than 30:1 and binding is nearly 100%. Further, 599 peptide is also able to bind to an siRNA that targets the HPV E6 oncogene, siE6, with a similar efficiency, particularly at a 50:1 peptide:siRNA molar ratio (FIG. 12B). Moreover, release of siE6 using heparin (FIG. 12C) and protection from RNAse A (FIG. 12D) when bound to 599 is comparable to results observed for siCIP2A complexed to 599 and its derivatives, particularly at a 50:1 peptide:siRNA molar ratio.

These results suggest that 599 peptide is more broadly applicable to a wider range of siRNA species than siCIP2A alone. One of skill in the art could thus appreciate how 599, and the derivatives that have been described herein, could be used in conjunction with a range of different siRNA gene-targeting sequences (and in the presence or absence of a second targeting peptide) to treat or prevent a variety of diseases or disorders.

### Example 5: 599 Peptide and Alternate Cargo

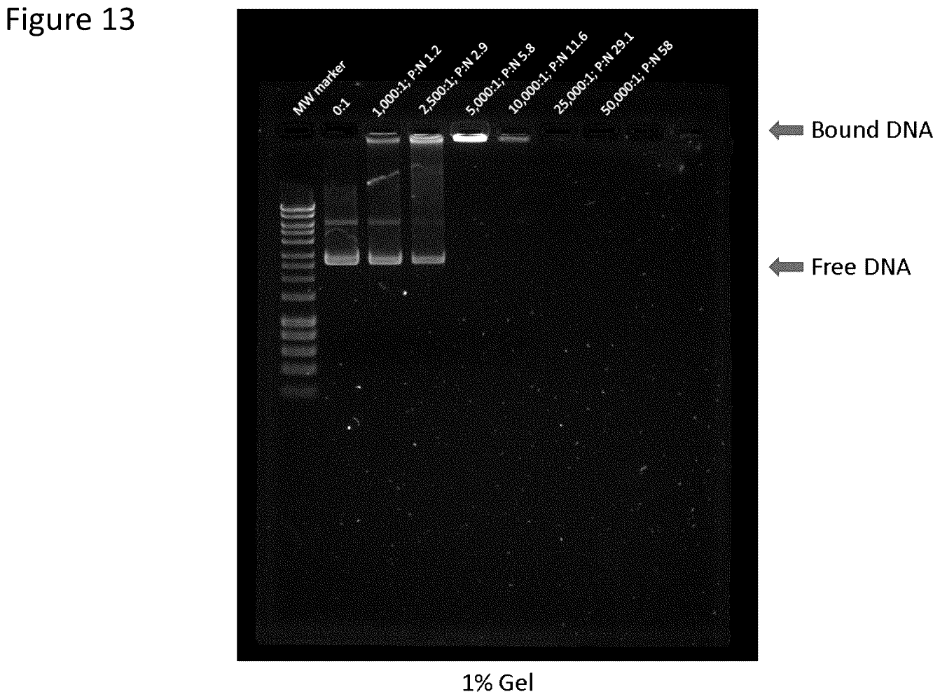

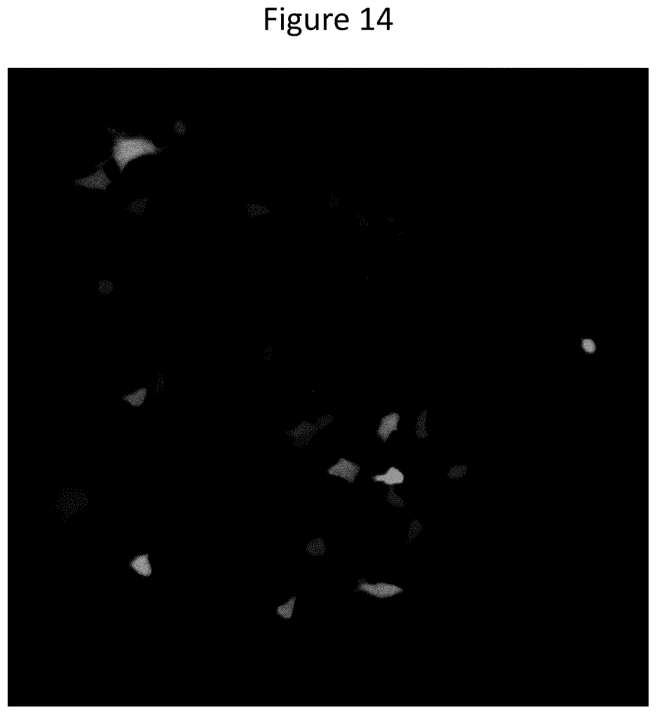

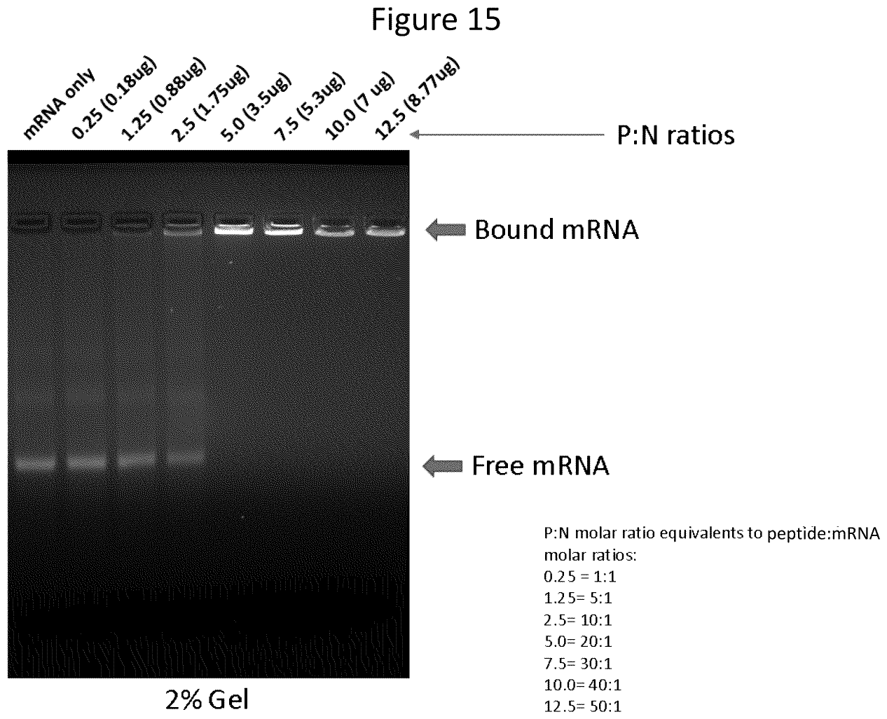

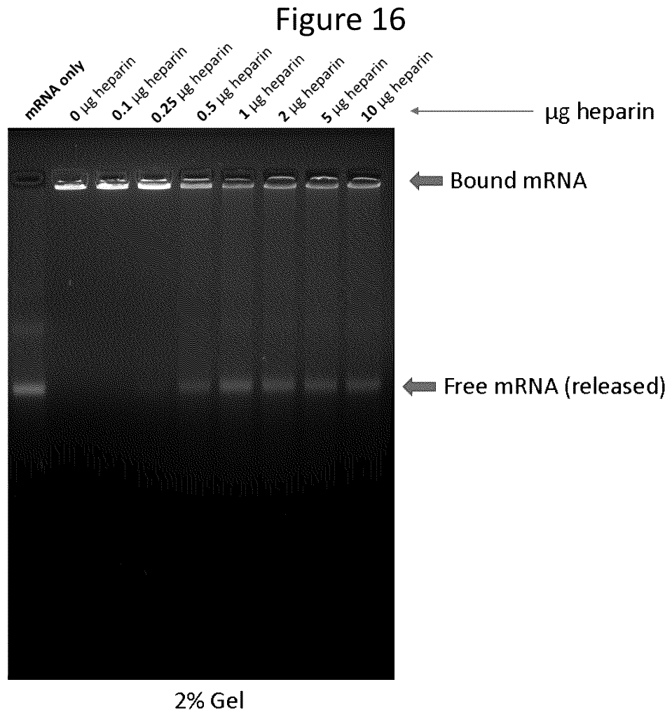

As the ability of 599 peptide and its derivatives to bind to siRNAs was demonstrated, it was next evaluated whether alternate cargo could be delivered via the peptides disclosed herein. First, it was demonstrated that 599 peptide had the ability to bind to plasmid DNA. Plasmid DNA 4.3 kb in length was shown to bind to 599 peptide, with the greatest binding observed at a peptide:DNA molar ratio of 5,000:1 (FIG. 13). Further, it was shown that the 599:plasmid DNA complex could enter cells and result in plasmid DNA expression, as evidenced by the expression of a GFP reporter encoded by the plasmid (FIG. 14). Second, it was demonstrated that 599 peptide had the ability to bind to mRNA, particularly above a 20:1 peptide:mRNA molar ratio (FIG. 15). A release assay also demonstrated that the 599 peptide has the ability to release its bound mRNA cargo upon addition of increasing amounts of heparin (FIG. 16). Thus, these results suggest that the compositions of the present invention, including 599 peptides and their derivatives, can be used to deliver additional cargo, such as plasmid DNA and mRNA, to cells for therapeutic and prophylactic benefit.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A composition for delivery of an agent comprising:
a) a peptide, wherein the peptide comprises a derivative of 599 peptide (SEQ ID NO: 1); and
b) an agent.

2. The composition of claim 1, wherein the peptide comprising a derivative of SEQ ID NO: 1 comprises an amino acid sequence in which at least one D-arginine residue of SEQ ID NO: 1 is substituted with an alanine residue.

3. The composition of claim 2, wherein at least one D-arginine residue of SEQ ID NO: 1 is substituted with a D-alanine residue.

4. The composition of claim 3, wherein at least one D-arginine residue of SEQ ID NO: 1 is substituted with a D-alanine residue at an amino acid residue position number selected from the group consisting of: 27, 28, 29, 30, 31, 32, 33, 34, and 35.

5. The composition of claim 4, wherein the peptide comprises an amino acid sequence selected from the group consisting of SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, and SEQ ID NO: 32.

6. The composition of claim 1, wherein the peptide comprising a derivative of SEQ ID NO: 1 comprises an amino acid sequence in which at least one D-arginine residue of SEQ ID NO: 1 is substituted with an L-alanine residue.

7. The composition of claim 6, wherein the at least one D-arginine residue of SEQ ID NO: 1 is substituted with an L-alanine residue at an amino acid residue position number selected from the group consisting of: 27, 28, 29, 30, 31, 32, 33, 34, and 35.

8. The composition of claim 7, wherein the peptide comprises an amino acid sequence selected from the group consisting of SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 34, SEQ ID NO: 35, SEQ ID NO: 36, SEQ ID NO: 37, SEQ ID NO: 38, SEQ ID NO: 39, and SEQ ID NO: 40.

9. The composition of claim 1, wherein the peptide comprising a derivative of SEQ ID NO: 1 comprises an amino acid sequence in which at least one D-arginine residue is substituted with an L-arginine residue.

10. The composition of claim 1, wherein the peptide comprising a derivative of SEQ ID NO: 1 comprises an amino acid sequence in which at least three D-arginine residues are each substituted with an L-arginine residue.

11. The composition of claim 10, wherein the peptide comprises an amino acid sequence selected from the group consisting of: SEQ ID NO: 2, SEQ ID NO: 3, SEQ ID NO: 4, SEQ ID NO: 5, SEQ ID NO: 6, SEQ ID NO: 8, SEQ ID NO: 9, SEQ ID NO: 10, SEQ ID NO: 11, SEQ ID NO: 12, SEQ ID NO: 13, and SEQ ID NO: 15.

12. The composition of claim 1, wherein the peptide comprising a derivative of SEQ ID NO: 1 comprises an amino acid sequence in which at least one L-form residue of SEQ ID NO: 1 is substituted with the D-form of said residue.

13. The composition of claim 12, wherein the peptide comprises an amino acid sequence selected from the group consisting of SEQ ID NO: 7 and SEQ ID NO: 14.

14. The composition of claim 1, wherein the agent is selected from the group consisting of: a nucleic acid molecule, a protein, a peptide, an antibody, an antibody fragment, and a small molecule.

15. The composition of claim 14, wherein the nucleic acid molecule is selected from the group consisting of: siRNA, microRNA, shRNA, antisense nucleic acid, ribozyme, killer-tRNAs, guide RNA (part of the CRISPR/CAS system), long non-coding RNA, anti-miRNA oligonucleotide, mRNA, and plasmid DNA.

16. The composition of claim 1, wherein the peptide and agent are at a molar ratio between about 5:1 to 50:1.

17. The composition of claim 1, wherein said peptide comprises a polyethylene glycol (PEG) modification to prevent renal clearance.

18. A method of administering an agent into a cell, the method comprising contacting the cell with an effective amount of a composition comprising:
a) a peptide, wherein the peptide comprises a derivative of 599 peptide (SEQ ID NO: 1); and
b) an agent.

19. A method of treating a disease or disorder in a subject in need thereof, the method comprising administering to the subject a therapeutically effective amount of a composition comprising:
a.) a peptide, wherein the peptide comprises a derivative of 599 peptide (SEQ ID NO: 1); and
b) an agent.

20. The method of claim 19, wherein the agent alleviates at least one symptom of the disease or disorder.

21. The method of claim 19, wherein the disease or disorder is cancer.

22. The composition of claim 1, further comprising a second peptide comprising a targeting moiety and a stretch of densely packed cationic amino acid residues.

23. The composition of claim 22, wherein the second peptide comprises an amino acid sequence of SEQ ID NO: 41.

24. A method of treating a disease or disorder in a subject in need thereof, the method comprising administering to the subject a therapeutically effective amount of the composition of claim 22.

25. A composition for delivery of an agent comprising:
a) a peptide, wherein the peptide comprises 599 peptide (SEQ ID NO: 1); and
b) an agent, wherein the agent comprises one or more selected from the group consisting of: plasmid DNA and mRNA.

26. A method of administering an agent to a cell, the method comprising contacting the cell with an effective amount of the composition of claim 25.

27. A method of treating a disease or disorder in a subject in need thereof, the method comprising administering to the subject a therapeutically effective amount of a composition comprising a peptide and an agent according to the methods of claim 26.


SEQUENCE LISTING (41 sequences)

SEQ ID NO: 1 - protein, 36 aa, Artificial Sequence
  Chemically synthesized peptide
  MISC_FEATURE (27)..(35): D-amino acids; all other amino acids not specified herein are L-amino acids or lack chirality.
GLFEAIEGFIENGWEGMIDGWYGGGGRRRRRRRRRK

SEQ ID NO: 2 - protein, 36 aa, Artificial Sequence; identical to SEQ ID NO: 1
  Chemically synthesized peptide
  MISC_FEATURE (30)..(32): D-amino acids; all other amino acids not specified herein are L-amino acids or lack chirality.

SEQ ID NO: 3 - protein, 36 aa, Artificial Sequence; identical to SEQ ID NO: 1
  Chemically synthesized peptide

SEQ ID NO: 4 - protein, 36 aa, Artificial Sequence; identical to SEQ ID NO: 1
  Chemically synthesized peptide
  misc_feature (27)..(27): D-amino acids; all other amino acids not specified herein are L-amino acids or lack chirality.
  misc_feature (35)..(35): D-amino acids; all other amino acids not specified herein are L-amino acids or lack chirality.

SEQ ID NO: 5 - protein, 36 aa, Artificial Sequence; identical to SEQ ID NO: 1
  Chemically synthesized peptide
  misc_feature (27)..(29): D-amino acids; all other amino acids not specified herein are L-amino acids or lack chirality.
  misc_feature (33)..(35): D-amino acids; all other amino acids not specified herein are L-amino acids or lack chirality.

SEQ ID NO: 6 - protein, 36 aa, Artificial Sequence; identical to SEQ ID NO: 1
  Chemically synthesized peptide
  misc_feature (27)..(27): D-amino acids; all other amino acids not specified herein are L-amino acids or lack chirality.
  misc_feature (29)..(29): D-amino acids; all other amino acids not specified herein are L-amino acids or lack chirality.
  misc_feature (31)..(31): D-amino acids; all other amino acids not specified herein are L-amino acids or lack chirality.
  misc_feature (33)..(33): D-amino acids; all other amino acids not specified herein are L-amino acids or lack chirality.
  misc_feature (35)..(35): D-amino acids; all other amino acids not specified herein are L-amino acids or lack chirality.

SEQ ID NO: 7 - protein, 36 aa, Artificial Sequence; identical to SEQ ID NO: 1
  Chemically synthesized peptide
  misc_feature (1)..(36): D-amino acids; all other amino acids not specified herein are L-amino acids or lack chirality.

SEQ ID NO: 8 - protein, 36 aa, Artificial Sequence; identical to SEQ ID NO: 1
  Chemically synthesized peptide
  misc_feature (1)..(22): D-amino acids; all other amino acids not specified herein are L-amino acids or lack chirality.

SEQ ID NO: 9 - protein, 36 aa, Artificial Sequence
  Chemically synthesized peptide
  misc_feature (29)..(32): D-amino acids; all other amino acids not specified herein are L-amino acids or lack chirality.
GLFEAIEGFIENGWEGMIDGWYGGGGRRARRRRRRK

SEQ ID NO: 10 - protein, 36 aa, Artificial Sequence; identical to SEQ ID NO: 9
  Chemically synthesized peptide
  misc_feature (29)..(29): D-amino acids; all other amino acids not specified herein are L-amino acids or lack chirality.

SEQ ID NO: 11 - protein, 36 aa, Artificial Sequence; identical to SEQ ID NO: 9
  Chemically synthesized peptide
  misc_feature (27)..(27): D-amino acids; all other amino acids not specified herein are L-amino acids or lack chirality.
  misc_feature (29)..(29): D-amino acids; all other amino acids not specified herein are L-amino acids or lack chirality.
  misc_feature (35)..(35): D-amino acids; all other amino acids not specified herein are L-amino acids or lack chirality.

SEQ ID NO: 12 - protein, 36 aa, Artificial Sequence; identical to SEQ ID NO: 9
  Chemically synthesized peptide
  misc_feature (27)..(29): D-amino acids; all other amino acids not specified herein are L-amino acids or lack chirality.
  misc_feature (33)..(35): D-amino acids; all other amino acids not specified herein are L-amino acids or lack chirality.

SEQ ID NO: 13 - protein, 36 aa, Artificial Sequence; identical to SEQ ID NO: 9
  Chemically synthesized peptide
  misc_feature (27)..(27): D-amino acids; all other amino acids not specified herein are L-amino acids or lack chirality.
  misc_feature (29)..(29): D-amino acids; all other amino acids not specified herein are L-amino acids or lack chirality.
  misc_feature (31)..(31): D-amino acids; all other amino acids not specified herein are L-amino acids or lack chirality.
  misc_feature (33)..(33): D-amino acids; all other amino acids not specified herein are L-amino acids or lack chirality.
  misc_feature (35)..(35): D-amino acids; all other amino acids not specified herein are L-amino acids or lack chirality.

SEQ ID NO: 14 - protein, 36 aa, Artificial Sequence; identical to SEQ ID NO: 9
  Chemically synthesized peptide
  misc_feature (1)..(36): D-amino acids; all other amino acids not specified herein are L-amino acids or lack chirality.

SEQ ID NO: 15 - protein, 36 aa, Artificial Sequence; identical to SEQ ID NO: 9
  Chemically synthesized peptide
  misc_feature (1)..(22): D-amino acids; all other amino acids not specified herein are L-amino acids or lack chirality.
  misc_feature (29)..(29): D-amino acids; all other amino acids not specified herein are L-amino acids or lack chirality.

SEQ ID NO: 16 - protein, 36 aa, Artificial Sequence
  Chemically synthesized peptide
  misc_feature (27)..(35): D-amino acids; all other amino acids not specified herein are L-amino acids or lack chirality.
GLFEAIEGFIENGWEGMIDGWYGGGGARRRRRRRRK

SEQ ID NO: 17 - protein, 36 aa, Artificial Sequence; identical to SEQ ID NO: 16
  Chemically synthesized peptide
  misc_feature (28)..(35): D-amino acids; all other amino acids not specified herein are L-amino acids or lack chirality.

SEQ ID NO: 18 - protein, 36 aa, Artificial Sequence
  Chemically synthesized peptide
  misc_feature (27)..(35): D-amino acids; all other amino acids not specified herein are L-amino acids or lack chirality.
GLFEAIEGFIENGWEGMIDGWYGGGGRARRRRRRRK

SEQ ID NO: 19 - protein, 36 aa, Artificial Sequence; identical to SEQ ID NO: 18
  Chemically synthesized peptide
  misc_feature (27)..(27): D-amino acids; all other amino acids not specified herein are L-amino acids or lack chirality.
  misc_feature (29)..(35): D-amino acids; all other amino acids not specified herein are L-amino acids or lack chirality.

SEQ ID NO: 20 - protein, 36 aa, Artificial Sequence; identical to SEQ ID NO: 9
  Chemically synthesized peptide
  misc_feature (27)..(35): D-amino acids; all other amino acids not specified herein are L-amino acids or lack chirality.

SEQ ID NO: 21 - protein, 36 aa, Artificial Sequence; identical to SEQ ID NO: 9
  Chemically synthesized peptide
  misc_feature (27)..(28): D-amino acids; all other amino acids not specified herein are L-amino acids or lack chirality.
  misc_feature (30)..(35): D-amino acids; all other amino acids not specified herein are L-amino acids or lack chirality.

SEQ ID NO: 22 - protein, 36 aa, Artificial Sequence
  Chemically synthesized peptide
  misc_feature (27)..(35): D-amino acids; all other amino acids not specified herein are L-amino acids or lack chirality.
GLFEAIEGFIENGWEGMIDGWYGGGGRRRARRRRRK

SEQ ID NO: 23 - protein, 36 aa, Artificial Sequence; identical to SEQ ID NO: 22
  Chemically synthesized peptide
  misc_feature (27)..(29): D-amino acids; all other amino acids not specified herein are L-amino acids or lack chirality.
  misc_feature (31)..(35): D-amino acids; all other amino acids not specified herein are L-amino acids or lack chirality.

SEQ ID NO: 24 - protein, 36 aa, Artificial Sequence
  Chemically synthesized peptide
  misc_feature (27)..(35): D-amino acids; all other amino acids not specified herein are L-amino acids or lack chirality.
GLFEAIEGFIENGWEGMIDGWYGGGGRRRRARRRRK

SEQ ID NO: 25 - protein, 36 aa, Artificial Sequence; identical to SEQ ID NO: 24
  Chemically synthesized peptide
  misc_feature (27)..(30): D-amino acids; all other amino acids not specified herein are L-amino acids or lack chirality.
  misc_feature (32)..(35): D-amino acids; all other amino acids not specified herein are L-amino acids or lack chirality.

SEQ ID NO: 26 - protein, 36 aa, Artificial Sequence
  Chemically synthesized peptide
  misc_feature (27)..(35): D-amino acids; all other amino acids not specified herein are L-amino acids or lack chirality.
GLFEAIEGFIENGWEGMIDGWYGGGGRRRRRARRRK

SEQ ID NO: 27 - protein, 36 aa, Artificial Sequence; identical to SEQ ID NO: 26
  Chemically synthesized peptide
  misc_feature (27)..(31): D-amino acids; all other amino acids not specified herein are L-amino acids or lack chirality.
  misc_feature (33)..(35): D-amino acids; all other amino acids not specified herein are L-amino acids or lack chirality.

SEQ ID NO: 28 - protein, 36 aa, Artificial Sequence
  Chemically synthesized peptide
  misc_feature (27)..(35): D-amino acids; all other amino acids not specified herein are L-amino acids or lack chirality.
GLFEAIEGFIENGWEGMIDGWYGGGGRRRRRRARRK

SEQ ID NO: 29 - protein, 36 aa, Artificial Sequence; identical to SEQ ID NO: 28
  Chemically synthesized peptide
  misc_feature (27)..(32): D-amino acids; all other amino acids not specified herein are L-amino acids or lack chirality.
  misc_feature (34)..(35): D-amino acids; all other amino acids not specified herein are L-amino acids or lack chirality.

SEQ ID NO: 30 - protein, 36 aa, Artificial Sequence
  Chemically synthesized peptide
  misc_feature (27)..(35): D-amino acids; all other amino acids not specified herein are L-amino acids or lack chirality.
GLFEAIEGFIENGWEGMIDGWYGGGGRRRRRRRARK

SEQ ID NO: 31 - protein, 36 aa, Artificial Sequence; identical to SEQ ID NO: 30
  Chemically synthesized peptide
  misc_feature (27)..(33): D-amino acids; all other amino acids not specified herein are L-amino acids or lack chirality.
  misc_feature (35)..(35): D-amino acids; all other amino acids not specified herein are L-amino acids or lack chirality.

SEQ ID NO: 32 - protein, 36 aa, Artificial Sequence
  Chemically synthesized peptide
  misc_feature (27)..(35): D-amino acids; all other amino acids not specified herein are L-amino acids or lack chirality.
GLFEAIEGFIENGWEGMIDGWYGGGGRRRRRRRRAK

SEQ ID NO: 33 - protein, 36 aa, Artificial Sequence; identical to SEQ ID NO: 32
  Chemically synthesized peptide
  misc_feature (27)..(34): D-amino acids; all other amino acids not specified herein are L-amino acids or lack chirality.

SEQ ID NO: 34 - protein, 36 aa, Artificial Sequence; identical to SEQ ID NO: 9
  Chemically synthesized peptide
  misc_feature (30)..(32): D-amino acids; all other amino acids not specified herein are L-amino acids or lack chirality.

SEQ ID NO: 35 - protein, 36 aa, Artificial Sequence; identical to SEQ ID NO: 9
  Chemically synthesized peptide

SEQ ID NO: 36 - protein, 36 aa, Artificial Sequence; identical to SEQ ID NO: 9
  Chemically synthesized peptide
  misc_feature (27)..(27): D-amino acids; all other amino acids not specified herein are L-amino acids or lack chirality.
  misc_feature (35)..(35): D-amino acids; all other amino acids not specified herein are L-amino acids or lack chirality.

SEQ ID NO: 37 - protein, 36 aa, Artificial Sequence; identical to SEQ ID NO: 9
  Chemically synthesized peptide
  misc_feature (27)..(28): D-amino acids; all other amino acids not specified herein are L-amino acids or lack chirality.
  misc_feature (33)..(35): D-amino acids; all other amino acids not specified herein are L-amino acids or lack chirality.

SEQ ID NO: 38 - protein, 36 aa, Artificial Sequence; identical to SEQ ID NO: 9
  Chemically synthesized peptide
  misc_feature (27)..(27): D-amino acids; all other amino acids not specified herein are L-amino acids or lack chirality.
  misc_feature (31)..(31): D-amino acids; all other amino acids not specified herein are L-amino acids or lack chirality.
  misc_feature (33)..(33): D-amino acids; all other amino acids not specified herein are L-amino acids or lack chirality.
  misc_feature (35)..(35): D-amino acids; all other amino acids not specified herein are L-amino acids or lack chirality.

SEQ ID NO: 39 - protein, 36 aa, Artificial Sequence; identical to SEQ ID NO: 9
  Chemically synthesized peptide
  misc_feature (1)..(28): D-amino acids; all other amino acids not specified herein are L-amino acids or lack chirality.
  misc_feature (30)..(36): D-amino acids; all other amino acids not specified herein are L-amino acids or lack chirality.

SEQ ID NO: 40 - protein, 36 aa, Artificial Sequence; identical to SEQ ID NO: 9
  Chemically synthesized peptide
  misc_feature (1)..(22): D-amino acids; all other amino acids not specified herein are L-amino acids or lack chirality.

SEQ ID NO: 41 - protein, 26 aa, Artificial Sequence
  Chemically synthesized peptide
YHWYGYTPQNVIGGGGRRRRRRRRRK